SEQUENCE LISTING (44 sequences)

SEQ ID NO: 1 - DNA, 66 bp, Artificial sequence
  oligonucleotide top strand
agcttgcggccgcgggcccagcggtggtggacctcactgagaagctagaggcttccaccaaaggcc

SEQ ID NO: 2 - DNA, 58 bp, artificial sequence
  oligonucleotide bottom strand
acgccggcgcccgggtcgccaccacctggagtgactcttcgatctccgaaggtggttt

SEQ ID NO: 3 - DNA, 50 bp, artificial sequence
  Kozak seq top strand
agcttgccgccaccatgggcagtcggtgcgcgctggccctggcggtgctc

SEQ ID NO: 4 - DNA, 40 bp, artificial sequence
  Kozak sequence bottom strand
acggcggtggtacccgtcagccacgcgcgaccgggaccgc

SEQ ID NO: 5 - DNA, 38 bp, artificial sequence
  kozak sequence top strand
tcggccttgctgtgtcaggtctggagctctggggtgtt

SEQ ID NO: 6 - DNA, 46 bp, artificial sequence
  Kozak sequence bottom strand
cacgagagccggaacgacacagtccagacctcgagaccccacaagc

SEQ ID NO: 7 - protein, 864 aa, artificial sequence
  fusion protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDECDPSPCKNGGSCTDLENSYSCTCPPGFYGKICELSAMTCADGPCFNGGRCSDSPDGGYSCRCPVGYSGFNCEKKIDYCSSSPCSNGAKCVDLGDAYLCRCQAGFSGRHCDDNVDDCASSPCANGGTCRDGVNDFSCTCPPGYTGRNCSAPVSRCEHAPCHNGATCHERGHGYVCECARGYGGPNCQFLLPELPPGPAVVDLTEKLEASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - DNA, 26 bp, artificial sequence
  PCR primer
caccatgggcagtcggtgcgcgctgg

SEQ ID NO: 9 - DNA, 45 bp, Artificial sequence
  PCR primer
gtctacgtttaaacttaacactcgtcaatccccagctcgcaggtg

SEQ ID NO: 10 - DNA, 999 bp, artificial sequence
  D1E3cys clone
atgggcagtcggtgcgcgctggccctggcggtgctctcggccttgctgtgtcaggtctggagctctggggtgttcgaactgaagctgcaggagttcgtcaacaagaaggggctgctggggaaccgcaactgctgccgcgggggcgcggggccaccgccgtgcgcctgccggaccttcttccgcgtgtgcctcaagcactaccaggccagcgtgtcccccgagccgccctgcacctacggcagcgccgtcacccccgtgctgggcgtcgactccttcagtctgcccgacggcgggggcgccgactccgcgttcagcaaccccatccgcttccccttcggcttcacctggccgggcaccttctctctgattattgaagctctccacacagattctcctgatgacctcgcaacagaaaacccagaaagactcatcagccgcctggccacccagaggcacctgacggtgggcgaggagtggtcccaggacctgcacagcagcggccgcacggacctcaagtactcctaccgcttcgtgtgtgacgaacactactacggagagggctgctccgttttctgccgtccccgggacgatgccttcggccacttcacctgtggggagcgtggggagaaagtgtgcaaccctggctggaaagggccctactgcacagagccgatctgcctgcctggatgtgatgagcagcatggattttgtgacaaaccaggggaatgcaagtgcagagtgggctggcagggccggtactgtgacgagtgtatccgctatccaggctgtctccatggcacctgccagcagccctggcagtgcaactgccaggaaggctgggggggccttttctgcaaccaggacctgaactactgcacacaccataagccctgcaagaatggagccacctgcaccaacacgggccaggggagctacacttgctcttgccggcctgggtacacaggtgccacctgcgagctggggattgacgagtgttaa

SEQ ID NO: 11 - protein, 332 aa, artificial sequence
  D1E3cys clone protein
MGSRCALALAVLSALLCQVWSSGVFELKLQEFVNKKGLLGNRNCCRGGAGPPPCACRTFFRVCLKHYQASVSPEPPCTYGSAVTPVLGVDSFSLPDGGGADSAFSNPIRFPFGFTWPGTFSLIIEALHTDSPDDLATENPERLISRLATQRHLTVGEEWSQDLHSSGRTDLKYSYRFVCDEHYYGEGCSVFCRPRDDAFGHFTCGERGEKVCNPGWKGPYCTEPICLPGCDEQHGFCDKPGECKCRVGWQGRYCDECIRYPGCLHGTCQQPWQCNCQEGWGGLFCNQDLNYCTHHKPCKNGATCTNTGQGSYTCSCRPGYTGATCELGIDEC

SEQ ID NO: 12 - protein, 43 aa, artificial sequence
  DSL consensus 1
CXXXXXXXXCXXXCXXXXXXXXXXXCXXXXXXXCXXXXXXXXC

SEQ ID NO: 13 - protein, 43 aa, artificial sequence; identical to SEQ ID NO: 12
  DSL consensus 2

SEQ ID NO: 14 - protein, 43 aa, artificial sequence
  DSL consensus 3
CXXXYYXXXCXXXCRPRXDXFGHXXCXXXGXXXCXXGWXGXXC

SEQ ID NO: 15 - protein, 18 aa, artificial sequence
  EGF-like domain consensus
XCXCXCXCXCXGXXGXCX

SEQ ID NO: 16 - DNA, 20 bp, artificial sequence
  PCR primer
gtaacccgttgaaccccatt

SEQ ID NO: 17 - DNA, 20 bp, artificial sequence
  PCR primer
ccatccaatcggtagtagcg

SEQ ID NO: 18 - DNA, 20 bp, artificial sequence
  PCR primer
ggtgctgataacagcggaat

SEQ ID NO: 19 - DNA, 20 bp, artificial sequence
  PCR primer
atttttggaatccttcacgc

SEQ ID NO: 20 - DNA, 26 bp, artificial sequence
  PCR primer
gatctggggggctataaaagggggta

SEQ ID NO: 21 - DNA, 26 bp, artificial sequence
  PCR primer
accccccgatattttcccccattcga

SEQ ID NO: 22 - DNA, 50 bp, artificial sequence
  TP1 promoter top strand
gatcccgactcgtgggaaaatgggcggaagggcaccgtgggaaaatagta

SEQ ID NO: 23 - DNA, 50 bp, artificial sequence
  TPI promoter bottom strand
ggctgagcacccttttacccgccttcccgtggcacccttttatcatctag

SEQ ID NO: 24 - protein, 63 aa, Drosophila sp.
WKTNKSESQYTSLEYDFRVTCDLNYYGSGCAKFCRPRDDSFGHSTCSETGEIICLTGWQGDYC

SEQ ID NO: 25 - protein, 63 aa, Homo sapiens
WSQDLHSSGRTDLKYSYRFVCDEHYYGEGCSVFCRPRDDAFGHFTCGERGEKVCNPGWKGPYC

SEQ ID NO: 26 - protein, 63 aa, Mus musculus
WSQDLHSSGRTDLRYSYRFVCDEHYYGEGCSVFCRPRDDAFGHFTCGDRGEKMCDPGWKGQYC

SEQ ID NO: 27 - protein, 63 aa, Rattus rattus
WSQDLHSSGRTDLRYSYRFVCDEHYYGEGCSVFCRPRDDAFGHFTCGERGEKMCDPGWKGQYC

SEQ ID NO: 28 - protein, 63 aa, Mus musculus
WRTDEQNDTLTRLSYSYRVICSDNYYGESCSRLCKKRDDHFGHYECQPDGSLSCLPGWTGKYC

SEQ ID NO: 29 - protein, 63 aa, Homo sapiens
WLLDEQTSTLTRLRYSYRVICSDNYYGDNCSRLCKKRNDHFGHYVCQPDGNLSCLPGWTGEYC

SEQ ID NO: 30 - protein, 63 aa, Rattus rattus
WQTLKQNTGIAHFEYQIRVTCDDHYYGFGCNKFCRPRDDFFGHYACDQNGNKTCMEGWMGPEC

SEQ ID NO: 31 - protein, 63 aa, Mus musculus
WQTLKQNTGIAHFEYQIRVTCDDHYYGFGCNKFCRPRDDFFGHYACDQNGNKTCMEGWMGPDC

SEQ ID NO: 32 - protein, 63 aa, Homo sapiens
WQTLKQNTGVAHFEYQIRVTCDDYYYGFGCNKFCRPRDDFFGHYACDQNGNKTCMEGWMGREC

SEQ ID NO: 33 - protein, 63 aa, Gallus sp.
WQTLKHNTGAAHFEYQIRVTCAEHYYGFGCNKFCRPRDDFFTHHTCDQNGNKTCLEGWTGPEC

SEQ ID NO: 34 - protein, 63 aa, Gallus sp.
WKTLQFNGPVANFEVQIRVKCDENYYSALCNKFCGPRDDFVGHYTCDQNGNKACMEGWMGEEC

SEQ ID NO: 35 - protein, 63 aa, Mus musculus
WKSLHFSGHVAHLELQIRVRCDENYYSATCNKFCRPRNDFFGHYTCDQYGNKACMDGWMGKEC

SEQ ID NO: 36 - protein, 63 aa, Homo sapiens; identical to SEQ ID NO: 35

SEQ ID NO: 37 - protein, 63 aa, Rattus rattus; identical to SEQ ID NO: 35

SEQ ID NO: 38 - protein, 63 aa, Homo sapiens; identical to SEQ ID NO: 35

SEQ ID NO: 39 - protein, 63 aa, Drosophila sp.
WKTLDHIGRNARITYRVRVQCAVTYYNTTCTTFCRPRDDQFGHYACGSEGQKLCLNGWQGVNC

SEQ ID NO: 40 - protein, 723 aa, Homo sapiens [sequence omitted]

SEQ ID NO: 41 - protein, 618 aa, Homo sapiens
MVSPRMSGLLSQTVILALIFLPQTRPAGVFELQIHSFGPGPGPGAPRSPCSARLPCRLFFRVCLKPGLSEEAAESPCALGAALSARGPVYTEQPGAPAPDLPLPDGLLQVPFRDAWPGTFSFIIETWREELGDQIGGPAWSLLARVAGRRRLAAGGPWARDIQRAGAWELRFSYRARCEPPAVGTACTRLCRPRSAPSRCGPGLRPCAPLEDECEAPLVCRAGCSPEHGFCEQPGECRCLEGWTGPLCTVPVSTSSCLSPRGPSSATTGCLVPGPGPCDGNPCANGGSCSETPRSFECTCPRGFYGLRCEVSGVTCADGPCFNGGLCVGGADPDSAYICHCPPGFQGSNCEKRVDRCSLQPCRNGGLCLDLGHALRCRCRAGFAGPRCEHDLDDCAGRACANGGTCVEGGGAHRCSCALGFGGRDCRERADPCAARPCAHGGRCYAHFSGLVCACAPGYMGARCEFPVHPDGASALPAAPPGLRPGDPQRYLLPPALGLLVAAGVAGAALLLVHVRRRGHSQDAGSRLLAGTPEPSVHALPDALNNLRTQEGSGDGPSSSVDWNRPEDVDPQGIYVISAPSIYAREVATPLFPPLHTGRAGQRQHLLFPYPSSILSVK

SEQ ID NO: 42 - protein, 685 aa, Homo sapiens
MAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 43 - protein, 1218 aa, Homo sapiens
MRSPRTRGRSGRPLSLLLALLCALRAKVCGASGQFELEILSMQNVNGELQNGNCCGGARNPGDRKCTRDECDTYFKVCLKEYQSRVTAGGPCSFGSGSTPVIGGNTFNLKASRGNDRNRIVLPFSFAWPRSYTLLVEAWDSSNDTVQPDSIIEKASHSGMINPSRQWQTLKQNTGVAHFEYQIRVTCDDYYYGFGCNKFCRPRDDFFGHYACDQNGNKTCMEGWMGPECNRAICRQGCSPKHGSCKLPGDCRCQYGWQGLYCDKCIPHPGCVHGICNEPWQCLCETNWGGQLCDKDLNYCGTHQPCLNGGTCSNTGPDKYQCSCPEGYSGPNCEIAEHACLSDPCHNRGSCKETSLGFECECSPGWTGPTCSTNIDDCSPNNCSHGGTCQDLVNGFKCVCPPQWTGKTCQLDANECEAKPCVNAKSCKNLIASYYCDCLPGWMGQNCDININDCLGQCQNDASCRDLVNGYRCICPPGYAGDHCERDIDECASNPCLNGGHCQNEINRFQCLCPTGFSGNLCQLDIDYCEPNPCQNGAQCYNRASDYFCKCPEDYEGKNCSHLKDHCRTTPCEVIDSCTVAMASNDTPEGVRYISSNVCGPHGKCKSQSGGKFTCDCNKGFTGTYCHENINDCESNPCRNGGTCIDGVNSYKCICSDGWEGAYCETNINDCSQNPCHNGGTCRDLVNDFYCDCKNGWKGKTCHSRDSQCDEATCNNGGTCYDEGDAFKCMCPGGWEGTTCNIARNSSCLPNPCHNGGTCVVNGESFTCVCKEGWEGPICAQNTNDCSPHPCYNSGTCVDGDNWYRCECAPGFAGPDCRININECQSSPCAFGATCVDEINGYRCVCPPGHSGAKCQEVSGRPCITMGSVIPDGAKWDDDCNTCQCLNGRIACSKVWCGPRPCLLHKGHSECPSGQSCIPILDDQCFVHPCTGVGECRSSSLQPVKTKCTSDSYYQDNCANITFTFNKEMMSPGLTTEHICSELRNLNILKNVSAEYSIYIACEPSPSANNEIHVAISAEDIRDDGNPIKEITDKIIDLVSKRDGNSSLIAAVAEVRVQRRPLKNRTDFLVPLLSSVLTVAWICCLVTAFYWCLRKRRKPGSHTHSASEDNTTNNVREQLNQIKNPIEKHGANTVPIKDYENKNSKMSKIRTHNSEVEEDDMDKHQQKARFAKQPAYTLVDREEKPPNGTPTKHPNWTNKQDNRDLESAQSLNRMEYIV

SEQ ID NO: 44 - protein, 1238 aa, Homo sapiens [sequence omitted]

(19) United States
(12) Patent Application Publication
(10) Pub. No.: US 2005/0220886 A1
(43) Pub. Date: Oct. 6, 2005

(54) Modulators of the Notch signalling pathway and uses thereof in medical treatment

(76) Inventors: Mark William Bodmer, Cambridge (GB); Emmanuel Cyrille Pascal Briend, Cambridge (GB); Brian Robert Champion, Cambridge (GB); Andrew Christopher Lennard, Cambridge (GB); Grahame James Mckenzie, Cambridge (GB); Tamara Tugal, Cambridge (GB); George Albert Ward, Cambridge (GB); Lesley Lynn Young, Cambridge (GB)

Correspondence Address: FROMMER LAWRENCE & HAUG, 745 FIFTH AVENUE- 10TH FL., NEW YORK, NY, 10151, US

(21) Appl. No.: 10/958,784

(22) Filed: Oct. 5, 2004

Related U.S. Application Data
(63) Continuation-in-part of application No. PCT/GB03/01525, filed on Apr. 4, 2003.

(30) Foreign Application Priority Data
Apr. 5, 2002 (GB) 0207930.9
Apr. 5, 2002 (GB) 0207929.1
May 28, 2002 (GB) 0212282.8
May 28, 2002 (GB) 0212283.6
Sep. 10, 2002 (GB) 0220913.8
Sep. 10, 2002 (GB) 0220912.0
Jan. 7, 2003 (GB) 0300234.2
Jul. 25, 2002 (WO) PCT/GB02/03426
Jul. 25, 2002 (WO) PCT/GB02/03397

(51) Int. Cl. A61K 9/14; A61K 38/17

(52) U.S. Cl. 424/489; 514/12

(57) ABSTRACT

A method is disclosed for therapeutic modulation of Notch signalling by administering a construct comprising a multiplicity of bond, linked or immobilised modulators of Notch signalling.

### CROSS-REFERENCE TO RELATED APPLICATIONS

This application is a continuation-in-part of International Application No. PCT/GB03/01525, filed Apr. 4, 2003, published as WO 03/087159 on Oct. 23, 2003, and claiming priority to GB application Serial Nos. 0207930.9 and 0207929.1, filed Apr. 5, 2002, 0212282.8 and 212283.6, filed May 28, 2002, 0220913.8 and 0220912.0, filed Sep. 10, 2002, and 030034.2, filed Jan. 7, 2003, and to International Application Nos. PCT/GB02/03397 and PCT/GB02/03426, filed Sep. 10, 2002.

Reference is made to U.S. application Ser. No. 09/310,685, filed May 4, 1999, Ser. No. 09/870,902, filed May 31, 2001, Ser. No. 10/013,310, filed Dec. 7, 2001, Ser. No. 10/147,354, filed May 16, 2002, Ser. No. 10/357,321, filed Feb. 3, 2002, Ser. No. 10/682,230, filed Oct. 9, 2003, Ser. No. 10/720,896, filed Nov. 24, 2003, Ser. No. 10/763,362, 10/764,415 and 10/765,727, all filed Jan. 23, 2004, Ser. No. 10/812,144, filed Mar. 29, 2004, Ser. No. 10/845,834, filed May 14, 2004 and 10/899,422, filed Jul. 26, 2004. Reference is also made to International Application No. PCT/GB02/05133, filed Nov. 13, 2002, and published as WO 03/042246 on May 22, 2003.

All of the foregoing applications, as well as all documents cited in the foregoing applications (“application documents”) and all documents cited or referenced in the application documents are incorporated herein by reference. Also, all documents cited in this application (“herein-cited documents”) and all documents cited or referenced in herein-cited documents are incorporated herein by reference. In addition, any manufacturer's instructions or catalogues for any products cited or mentioned in each of the application documents or herein-cited documents are incorporated by reference. Documents incorporated by reference into this text or any teachings therein can be used in the practice of this invention.

Documents incorporated by reference into this text are not admitted to be prior art.

### FIELD OF THE INVENTION

The present invention relates to therapeutic modulation of the Notch signalling pathway, particularly, but not exclusively, in immune cells.

### BACKGROUND OF THE INVENTION

Notch signal transduction plays a critical role in cell fate determination in vertebrate and invertebrate tissues. Notch is expressed at many stages of Drosophila embryonic and larval development and in many different cells implying a wide range of functions including an important role in neurogenesis and in the differentiation of mesodermal and endodermal cells. There are at least four mammalian Notch genes (Notch-1, Notch-2, Notch-3 and Notch-4). Notch-1, which most closely resembles the proteins of invertebrates and lower vertebrates, is widely expressed and is essential for early development. Recent evidence suggests that Notch signalling contributes to lineage commitment of immature T-cells in the thymus.

During maturation in the thymus, T-cells acquire the ability to distinguish self-antigens from those that are non-self, a process termed “self tolerance”. Tolerance to a non-self antigen, however, may be induced by immunisation under specific conditions with a peptide fragment comprising that antigen. In autoimmune diseases such as multiple sclerosis, rheumatoid arthritis or diabetes, there is a failure of the proper regulation of tolerance. Improved treatment methods for re-establishing tolerance are desirable for autoimmune diseases. Similarly in allergic conditions and for transplantation of an organ or tissue from a donor individual, induction of tolerance to particular foreign antigens or profiles of foreign antigens is desirable.

The expression on the cell surface of normal adult cells of the peripheral immune system of Notch and its ligands, Delta and Serrate, suggests a role for these proteins in T-cell acquired immunocompetence. T-cells express Notch-1 mRNA constitutively. Delta expression is limited to only a subset of T-cells in the peripheral lymphoid tissues. Serrate expression is restricted to a subset of antigen presenting cells (APCs). These observations reinforce the view that the Notch receptor ligand family continues to regulate cell fate decisions in the immune system beyond embryonic development with Notch signalling playing a central role in the induction of peripheral unresponsiveness (tolerance or anergy), linked suppression and infectious tolerance (Hoyne et al).

Thus, as described in WO 98/20142, manipulation of the Notch signalling pathway can be used in immunotherapy and in the prevention and/or treatment of T-cell mediated diseases. In particular, allergy, autoimmunity, graft rejection, tumour induced aberrations to the T-cell system and infectious diseases caused, for example, by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara, may be targeted.

It has also recently been shown that it is possible to generate a class of regulatory T cells which are able to transmit antigen-specific tolerance to other T cells, a process termed infectious tolerance (WO98/20142). The functional activity of these cells can be mimicked by over-expression of a Notch ligand protein on their cell surfaces or on the surface of antigen presenting cells. In particular, regulatory T cells can be generated by over-expression of a member of the Delta or Serrate family of Notch ligand proteins. Delta or Serrate induced T cells specific to one antigenic epitope are also able to transfer tolerance to T cells recognising other epitopes on the same or related antigens, a phenomenon termed “epitope spreading”.

Notch ligand expression also plays a role in cancer. Indeed, upregulated Notch ligand expression has been observed in some tumour cells. These tumour cells are capable of rendering T cells unresponsive to restimulation with a specific antigen, thus providing a possible explanation of how tumour cells prevent normal T cell responses. By downregulating Notch signalling in vivo in T cells, it may be possible to prevent tumour cells from inducing immunotolerance in those T cells that recognise tumour-specific antigens. In turn, this would allow the T cells to mount an immune response against the tumour cells (WO00/135990).

Varnum-Finney at al (Blood 1998, Vol 91, No 11, pp 4084-4091) describes how the effect of Jagged-1 on murine marrow precursor cells was assessed by co-culturing sorted precursor cells with a 3T3 cell layer that expressed human Jagged-1 or by incubating sorted precursors with beads coated with the purified extracellular domain of human Jagged-1.

The present invention seeks to provide methods, uses and compositions for modulating the Notch signalling pathway in therapy, and particularly for modulation of immune cell activity in immunotherapy.

Administration of modulators of Notch signalling according to the present invention provides improved activity, especially improved Notch signalling agonist activity.

### SUMMARY OF THE INVENTION

According to a first aspect of the invention there is provided a pharmaceutical composition comprising a construct which comprises a multiplicity of bound or linked modulators of Notch signalling. Suitably the pharmaceutical composition further comprises a pharmaceutically acceptable diluent or carrier. Suitably the composition is in sterile form, especially when used in vivo.

Suitably the modulators of Notch signalling are presented by the construct in an oreientation suitable for activation of a Notch receptor, and are preferably orientated on the surface of the construct.

The invention further provides a method for therapeutic modulation of Notch signalling by administering a construct comprising a multiplicity of bound or linked modulators of Notch signalling.

The invention further provides a method for generating a regulatory T-cell or increasing regulatory T-cell activity by contacting a construct comprising a multiplicity of bound or linked modulators of Notch signalling with a T-cell.

According to a further aspect of the invention there is provided a method for therapeutic modulation of Notch signalling in immune cells by administering a construct comprising a multiplicity of bound or linked modulators of Notch signalling.

According to a further aspect of the invention there is provided a method for therapeutic modulation of immune cell activity by administering a construct comprising a multiplicity of bound or linked modulators of Notch signalling.

According to a further aspect of the invention there is provided a method for therapeutic modulation of T-cell activity by administering a construct comprising a multiplicity of bound or linked modulators of Notch signalling.

According to a further aspect of the invention there is provided a method for treating inflammation, asthma, allergy, graft rejection, graft-versus-host disease or autoimmune disease by administering a construct comprising a multiplicity of bound or linked modulators of Notch signalling.

According to a further aspect of the invention there is provided a construct comprising a multiplicity of bound or linked modulators of Notch signalling for use in the treatment of disease.

According to a further aspect of the invention there is provided a construct comprising a multiplicity of bound or linked modulators of Notch signalling for use in the treatment of an immune disorder.

According to a further aspect of the invention there is provided a particle bearing a multiplicity of bound modulators of Notch signalling for use in the treatment of disease. Suitably the modulators of Notch signalling are presented on the particle in an orientation suitable for activation of a Notch receptor, and are preferably orientated on the surface of the particle.

According to a further aspect of the invention there is provided a particle bearing a multiplicity of bound modulators of Notch signalling for use in the treatment of an immune disorder.

According to a further aspect of the invention there is provided the use of a construct comprising a multiplicity of bound or linked modulators of Notch signalling in the manufacture of a medicament for modulation of immune cell activity. Preferably the immune cells are not stem cells.

According to a further aspect of the invention there is provided a method for treating an immune disorder by administering a construct comprising a multiplicity of bound or linked modulators of Notch signalling.

According to a further aspect of the invention there is provided a substrate bearing a multiplicity of bound modulators of Notch signalling for use in the treatment of disease.

According to a further aspect of the invention there is provided the use of a construct comprising a multiplicity of bound or linked modulators of Notch signalling for the manufacture of a medicament for modulation of expression of a cytokine selected from IL-10, IL-5, IL-2, TNF-alpha, IFN-gamma or IL-13.

Thus in one aspect there is provided the use of a construct comprising a multiplicity of bound or linked modulators of Notch signalling for the manufacture of a medicament for increase of IL-10 expression.

Alternatively there is provided the use of a construct comprising a multiplicity of bound or linked modulators of Notch signalling for the manufacture of a medicament for decrease of expression of a cytokine selected from IL-2, IL-5, TNF-alpha, IFN-gamma or IL-13.

According to a further aspect of the invention there is provided the use of a construct comprising a multiplicity of bound or linked modulators of Notch signalling for the manufacture of a medicament for generating an immune modulatory cytokine profile with increased IL-10 expression and reduced IL-5 expression.

According to a further aspect of the invention there is provided the use of a construct comprising a multiplicity of bound or linked modulators of Notch signalling for the manufacture of a medicament for generating an immune modulatory cytokine profile with increased IL-10 expression and reduced IL-2, IFN-gamma, IL-5, IL-13 and TNF-alpha expression.

In one embodiment the construct may be a substantially or partially water-soluble construct. Alternatively it may be a water insoluble or dispersable construct.

According to a further aspect of the invention there is provided a particle comprising a modulator of Notch signalling bound to a particulate support matrix. Suitably the particulate support matrix is a bead. Suitably a plurality of Notch ligands are bound to the particulate support matrix.

According to a further aspect of the invention there is provided a particle comprising a modulator of Notch signalling bound to a particulate support matrix. Suitably the particulate support matrix is a bead. Suitably the modulator of Notch signalling is a Notch ligand or fragment thereof. Suitably a plurality of Notch ligands are bound to the particulate support matrix.

According to a further aspect of the invention there is provided a method of modifying immune cell (e.g. T-cell) activity ex-vivo by contacting an immune cell with a surface (e.g., bead, well, plate) to which is bound (chemically or by affinity or adsporption) a Notch signalling agonist, such as a Notch ligand proein comprising a DSL domain and at least 2 EGF domains, and administering the cell to a patient.

The invention also provides a plate or well which is coated with a plurality of Notch signalling agonists, suitably the agonists may be coupled to the plate chemically.

According to a further aspect of the invention there is provided a pharmaceutically acceptable support matrix suitable for in vivo administration which bears a modulator of Notch signalling.

Suitably the support matrix may be in the form of an implantable support matrix, for example in the form of a particle. Suitably the support matrix bears a Notch ligand, and suitably bears a multiplicity of Notch ligands.

According to a further aspect of the invention there is provided a protein or polypeptide consisting essentially of the following components:
i) a Notch ligand DSL domain;
ii) 1-5 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide consisting essentially of the following components:
i) a Notch ligand DSL domain;
ii) 2-4 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide consisting essentially of the following components:
i) a Notch ligand DSL domain;
ii) 2-3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide consisting essentially of the following components:
i) a Notch ligand DSL domain;
ii) 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide comprising:
i) a Notch ligand DSL domain;
ii) 1-5 and no more than 5 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide comprising:
i) a Notch ligand DSL domain;
ii) 2-4 and no more than 4 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide comprising:
i) a Notch ligand DSL domain;
ii) 2-3 and no more than 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

According to a further aspect of the invention there is provided a protein or polypeptide comprising:
i) a Notch ligand DSL domain;
ii) 3 and no more than 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

In one embodiment the coupling agent is suitable for chemical coupling, such as a chemically reactive element.

Alternatively, the coupling agent may be suitable for adsorption coupling, for example suing electrostatic or hydrophobic interactions, or affinity coupling, for example using antibodies.

Suitably the coupling agent is at the C-terminus of the protein or polypeptide. In one embodiment the coupling agent is a C-terminal cysteine, aspartate or glutamate residue.

Suitably the protein or polypeptide has at least 50%, preferably at least 70%, preferably at least 90%, for example at least 95% amino acid sequence similarity (or preferably sequence identity) to the following sequence along the entire length of the latter:

| | |
|---|---|
| MGSRCALALAVLSALLCQVWSSGVFELKLQEFVNKKGLLGNRNCCRGGAGPPPCACRTF | (SEQ ID NO: 1) |
| FRVCLKHYQASVSPEPPCTYGSAVTPVLGVDSFSLPDGGGADSAFSNPIRFPFGFTWPG | |
| TFSLIIEALHTDSPDDLATENPERLISRLATQRHLTVGEEWSQDLHSSGRTDLKYSYRF | |
| VCDEHYYGEGCSVFCRPRDDAFGHFTCGERGEKVCNPGWKGPYCTEPICLPGCDEQHGF | |
| CDKPGECKCRVGWQGRYCDECIRYPGCLHGTCQQPWQCNCQEGWGGLFCNQDLNYCTHH | |
| KPCKNGATCTNTGQGSYTCSCRPGYTGATCELGIDEC | |

According to a further aspect of the invention there is provided a pharmaceutically acceptable support matrix bearing a multiplicity of proteins or polypeptides as described above, said proteins being coupled to the support matrix. Suitably the coupling may be chemical coupling, affinity coupling or adsorption coupling. Suitably the support matrix may be a particulate support matrix, preferably a bead, preferably a microbead or nanobead.

According to a further aspect of the invention there is provided a bead coupled to a protein or polypeptide as described above. Suitably the bead has a diameter of from about 0.001 to about 1000 micrometres.

Suitably the bead is a polymeric bead. In one embodiment the bead comprises a biodegradable material.

Suitably, for example, the bead comprises polystyrene, polyacrylamide, latex, cellulose, silica, dextran, agarose, cellulose, polylactide, or poly(methylmethacrylate) (PMMA) optionally in modified, crosslinked or derivatized form.

According to a further aspect of the invention there is provided a pharmaceutical composition, e.g. for in vivo use, comprising a particle or bead as described.

The term “plurality” as used herein means a number being at least two, and preferably at least five, suitably at least ten, twenty or more.

The term “multiplicity” as used herein means a number being at least three, and preferably at least five, suitably at least ten, for example at least twenty or a hundred or more.

Suitably the construct used in the various embodiments of the invention comprises at least 3, preferably at least 5, suitably at least 10, suitably at least 20, for example 100 or more modulators of Notch signalling which may be the same or different.

Suitably the construct comprises a multiplicity of modulators of Notch signalling bound to a substrate. Preferably the substrate is a particulate substrate, such as a bead. Suitably the substrate may be biodegradable, especially where used in vivo.

Suitably such a particle or bead has a diameter (or for a collection of particles or beads, an average diameter) of from about 0.001 to about 1000 micrometres, suitably from 0.01 to 100 micrometres. The particle or bead may suitably be a microbead or nanobead or microsphere or nanosphere.

Suitably the particle or bead is a polymeric particle or bead. For example, the particle or bead may comprise polystyrene, polyacrylamide, latex, cellulose, silica, dextran, agarose, cellulose, polylactide, or poly(methylmethacrylate) (PMMA) optionally in modified, crosslinked or derivatized form.

Suitably the particle or bead comprises a biodegradable material. Suitably the particle or bead is in sterile form.

Modulators of the Notch signalling pathway may be administered therapeutically on pharmaceutically acceptable support matrices.

Thus in one embodiment the invention provides a pharmaceutically acceptable support matrix suitable for in vivo administration which bears a modulator of Notch signalling.

Suitably, for example, the support matrix may be in the form of an implantable support matrix.

Alternatively, for example, the support matrix may be in the form of a particle or bead.

Suitably the support matrix may bear a Notch ligand proteins or polypeptides, preferably a multiplicity of Notch ligand proteins or polypeptides.

Such support matrices, particles, beads etc may be administered either in vivo or ex-vivo as well known in the art (for example as described herein under the heading “Pharmaceutical Compositions”) and used to modulate the Notch signalling pathway (for example to treat conditions as described herein under the heading “Therapy”).

Preferably the modulator of Notch signalling is an agent capable of activating Notch signalling. Preferably the agent is capable of activating Notch signalling in lymphocytes, preferably T-cells. For example, the agent may act to reduce activity of effector T-cells such as Th or Tc T-cells, and/or increase activity of regulatory T-cells.

Where a multiplicity of modulators is provided according to the invention, it will be appreciated that each individual modulator may be the same or different to each of the others.

In one embodiment the cells used in the present invention are not stem cells.

Preferably the modulator of Notch signalling is an agent capable of activating a Notch receptor, such as a Notch 1, Notch 2, Notch 3 or Notch 4 receptor. Suitably, for example, the modulator may be a Notch ligand or a biologically active fragment or derivative of a Notch ligand, or a peptidomimetic of such a Notch ligand. Preferably the agent is capable of activating Notch receptors in lymphocytes such as T-cells.

Suitably the modulator of the Notch signalling pathway may comprise or code for a fusion protein. For example, the modulator may comprise or code for a fusion protein comprising a segment of a Notch ligand extracellular domain and an immunoglobulin Fc segment.

Suitably the modulator of the Notch signalling pathway may comprise a fusion protein comprising a segment of a Notch ligand extracellular domain and an immunoglobulin Fc segment (e.g. IgG1 Fc or IgG4 Fc) or a polynucleotide coding for such a fusion protein. Suitable such fusion proteins are described, for example in Example 2 of WO 98/20142. IgG fusion proteins may be prepared as well known in the art, for example, as described in U.S. Pat. No. 5,428,130 (Genentech).

Suitably the modulator of the Notch signalling pathway comprises or codes for a protein or polypeptide comprising a Notch ligand DSL domain or a fragment, derivative, homologue, analogue or allelic variant thereof.

Preferably the modulator of the Notch signalling pathway comprises or codes for a Notch ligand DSL domain and at least one EGF-like domain, suitably at least 1 to 20, suitably at least 2 to 16, for example at least 2 to 10, for example from 2 to 5 EGF-like domains. Suitably the DSL and EGF sequences are or correspond to mammalian sequences. Preferred sequences include human sequences. Suitably the Notch ligand domains are fused to a heterologous amino acid sequence such as an IgFc sequence.

Alternatively or in addition the modulator of the Notch signalling pathway may comprise a Notch intracellular domain (Notch IC) or a fragment, derivative, homologue, analogue or allelic variant thereof, or a polynucleotide sequence which codes for Notch intracellular domain or a fragment, derivative, homologue, analogue or allelic variant thereof.

Suitably a modulator of the Notch signalling pathway comprises Delta or a fragment, derivative, homologue, analogue or allelic variant thereof or a polynucleotide encoding Delta or a fragment, derivative, homologue, analogue or allelic variant thereof.

Alternatively or in addition a modulator of the Notch signalling pathway may comprise Serrate/Jagged or a fragment, derivative, homologue, analogue or allelic variant thereof or a polynucleotide encoding Serrate/Jagged or a fragment, derivative, homologue, analogue or allelic variant thereof.

Alternatively or in addition a modulator of the Notch signalling pathway may comprise Notch or a fragment, derivative, homologue, analogue or allelic variant thereof or a polynucleotide encoding Notch or a fragment, derivative, homologue, analogue or allelic variant thereof.

Alternatively or in addition a modulator of the Notch signalling pathway may comprise a dominant negative version of a Notch signalling repressor, or a polynucleotide which codes for a dominant negative version of a Notch signalling repressor.

Alternatively or in addition a modulator of the Notch signalling pathway may comprise a polypeptide capable of upregulating the expression or activity of a Notch ligand or a downstream component of the Notch signalling pathway, or a polynucleotide which codes for such a polypeptide.

Suitably the modulator of the Notch signalling pathway may comprise an antibody, antibody fragment or antibody derivative or a polynucleotide which codes for an antibody, antibody fragment or antibody derivative. Thus the invention also provides a support (such as a bead, plate or well).

In one embodiment the modulator of Notch signalling may be administered in a multimerised form. For example, in one embodiment the modulator of Notch signalling may be bound to a membrane or support. Suitably a plurality or multiplicity of modulators (for example at least 5, 10, 20 or 100) will be bound to the membrane or support. Such a membrane or support can be selected from those known in the art. In one embodiment, the support may be a particulate support matrix. For example, the support may be a bead. The bead may be, for example, a magnetic bead (e.g. as available under the trade name “Dynal”).

In one embodiment the modulation of the immune system comprises reduction of T cell activity. For example, the modulation of the immune system may comprise reduction of effector T-cell activity, for example reduction of helper (T_{H}) and/or cytotoxic (T_{C}) T-cell activity. Suitably the modulation of the immune system may comprise reduction of a Th1 or Th2 immune response.

Alternatively or in addition, the modulation of the immune system provides an increase of regulatory T-cell (T reg) activity, such as an increase of Tr1 or Th3 regulatory T-cell activity.

Suitably the modulation of the immune system comprises generation of regulatory T cells (Tregs) and/or enhancement of Treg activity.

Suitably the modulation of the immune system comprises treatment of asthma, allergy, graft rejection, graft-versus-host disease or autoimmune disease.

According to a further aspect of the invention there is provided a method for producing a lymphocyte or antigen presenting cell (APC) capable of promoting tolerance which method comprises incubating a lymphocyte or APC obtained from a human or animal patient with a modulator of the Notch signalling pathway as described above.

Suitably the method comprises incubating a lymphocyte or APC obtained from a human or animal patient with an APC in the presence of a modulator of the Notch signalling pathway as described above.

According to a further aspect of the invention there is provided a method for producing an APC capable of inducing tolerance in a T cell which method comprises contacting an APC with a modulator of the Notch signalling pathway as described above.

According to a further aspect of the invention there is provided a method for producing a lymphocyte or APC capable of promoting tolerance which method comprises incubating a lymphocyte or APC obtained from a human or animal patient with a lymphocyte or APC produced as described above.

Suitably in such methods the lymphocyte or APC may be incubated either in vivo or ex-vivo.

According to a further aspect of the invention there is provided a particle (such as a bead, including microbeads and nanobeads) comprising a plurality, preferably a multiplicity of Delta proteins or polypeptides bound to a particulate support matrix. The term “Delta protein or polypeptide” as used herein suitably includes a protein or polypeptide which has at least one DSL domain from a Delta Notch ligand such as Delta1, Delta3 or Delta4, and suitably at least one, preferably at least two, for example 2 to 10 EGF-like domains from a Delta Notch ligand. Preferably the Delta Notch ligand is or is derived from a verterbrate, preferably a mammalian Notch ligand sequence, for example a Xenopus, mouse or human sequence.

### BRIEF DESCRIPTION OF THE DRAWINGS

Various preferred features and embodiments of the present invention will now be described by way of non-limiting examples and with reference to the accompanying drawings in which:

FIG. 1 shows a schematic representation of the Notch signalling pathway;

FIG. 2 shows schematic representations of the Notch ligands Jagged and Delta;

FIG. 3 shows aligned amino acid sequences of DSL domains from various Drosophila and mammalian Notch ligands (SEQ ID NOs:2-17);

FIGS. 4A-4C show the amino acid sequences of human Delta-1 (FIG. 4A; SEQ ID NO:18), Delta-3 (FIG. 4B; SEQ ID NO:19) and Delta-4 (FIG. 4C; SEQ ID NO:20);

FIGS. 5A and 5B show the amino acid sequences of human Jagged-1 (FIG. 5A; SEQ ID NO:21) and Jagged-2 (FIG. 5B; SEQ ID NO:22);

FIG. 6 shows schematic representations of various Notch ligand domain/IgFc domain fusion proteins which may be used in the present invention;

FIG. 7 shows a reaction scheme for covalently linking modulators of Notch signalling to beads.

FIG. 8 shows schematic representations of non-covalent linking of modulators of Notch signalling to beads (using a streptavidin/biotin link).

FIG. 9 shows the results of Example 3;

FIG. 10 shows the results of Example 4;

FIG. 11 shows the results of Example 5;

FIG. 12 shows the results of Example 6;

FIG. 13 shows the results of Example 7;

FIGS. 14A-14D show the results of Example 8;

FIG. 15 shows the results of Example 10;

FIGS. 16 to 18 show the results of Example 11;

FIG. 19 shows the results of Example 12;

FIG. 20 shows the results of Example 13;

FIG. 21 shows the results of Example 14;

FIG. 22 shows the results of Example 16;

FIG. 23 shows the results of Example 17;

FIG. 24 shows the results of Example 18;

FIG. 25 shows the results of Example 19;

FIGS. 26 to 33 show the results of Example 22;

FIG. 34 shows the results of Example 23;

FIG. 35 shows the results of Example 24; and

FIG. 36 shows the results of Example 25.

### DETAILED DESCRIPTION

The practice of the present invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; and, D. M. J. Lilley and J. E. Dahlberg, 1992, Methods of Enzymology: DNA Structure Part A: Synthesis and Physical Analysis of DNA Methods in Enzymology, Academic Press. Each of these general texts is herein incorporated by reference.

Notch Signalling

As used herein, the expression “Notch signalling” is synonymous with the expression “the Notch signalling pathway” and refers to any one or more of the upstream or downstream events that result in, or from, (and including) activation of the Notch receptor.

Notch signalling directs binary cell fate decisions in the embryo. Notch was first described in Drosophila as a transmembrane protein that functions as a receptor for two different ligands, Delta and Serrate. Vertebrates express multiple Notch receptors and ligands. At least four Notch receptors (Notch-1, Notch-2, Notch-3 and Notch-4) have been identified to date in human cells.

Notch proteins are synthesized as single polypeptide precursors that undergo cleavage via a Furin-like convertase that yields two polypeptide chains that are further processed to form the mature receptor. The Notch receptor present in the plasma membrane comprises a heterodimer of two Notch proteolytic cleavage products, one comprising an N-terminal fragment consisting of a portion of the extracellular domain, the transmembrane domain and the intracellular domain, and the other comprising the majority of the extracellular domain. The proteolytic cleavage step of Notch to activate the receptor occurs and is mediated by a furin-like convertase.

Notch receptors are inserted into the membrane as disulphide-linked heterodimeric molecules consisting of an extracellular domain containing up to 36 epidermal growth factor (EGF)-like repeats and a transmembrane subunit that contains the cytoplasmic domain. The cytoplasmic domain of Notch contains six ankyrin-like repeats, a polyglutamine stretch (OPA) and a PEST sequence. A further domain termed RAM23 lies proximal to the ankyrin repeats and, like the ankyrin-like repeats, is involved in binding to a transcription factor, known as Suppressor of Hairless [Su(H)] in Drosophila and CBF1 in vertebrates (Tamura). The Notch ligands also display multiple EGF-like repeats in their extracellular domains together with a cysteine-rich DSL (Delta-Serrate Lag2) domain that is characteristic of all Notch ligands (Artavanis-Tsakonas).

The Notch receptor is activated by binding of extracellular ligands, such as Delta, Serrate and Scabrous, to the EGF-like repeats of Notch's extracellular domain. Delta requires cleavage for activation. It is cleaved by the ADAM disintegrin metalloprotease Kuzbanian at the cell surface, the cleavage event releasing a soluble and active form of Delta. An oncogenic variant of the human Notch-1 protein, also known as TAN-1, which has a truncated extracellular domain, is constitutively active and has been found to be involved in T-cell lymphoblastic leukemias.

The cdc10/ankyrin intracellular-domain repeats mediate physical interaction with intracellular signal transduction proteins. Most notably, the cdc10/ankyrin repeats interact with Suppressor of Hairless [Su(H)]. Su(H) is the Drosophila homologue of C-promoter binding factor-1 [CBF-1], a mammalian DNA binding protein involved in the Epstein-Barr virus-induced immortalization of B-cells. It has been demonstrated that, at least in cultured cells, Su(H) associates with the cdc10/ankyrin repeats in the cytoplasm and translocates into the nucleus upon the interaction of the Notch receptor with its ligand Delta on adjacent cells. Su(H) includes responsive elements found in the promoters of several genes and has been found to be a critical downstream protein in the Notch signalling pathway. The involvement of Su(H) in transcription is thought to be modulated by Hairless.

The intracellular domain of Notch (NotchIC) also has a direct nuclear function (Lieber). Recent studies have indeed shown that Notch activation requires that the six cdc 10/ankyrin repeats of the Notch intracellular domain reach the nucleus and participate in transcriptional activation. The site of proteolytic cleavage on the intracellular tail of Notch has been identified between gly1743 and val1744 (termed site 3, or S3) (Schroeter). It is thought that the proteolytic cleavage step that releases the NotchIC for nuclear entry is dependent on Presenilin activity.

The intracellular domain has been shown to accumulate in the nucleus where it forms a transcriptional activator complex with the CSL family protein CBF1 (suppressor of hairless, Su(H) in Drosophila, Lag-2 in C. elegans) (Schroeter; Struhl). The NotchIC-CBF1 complexes then activate target genes, such as the bHLH proteins HES (hairy-enhancer of split like) 1 and 5 (Weinmaster). This nuclear function of Notch has also been shown for the mammalian Notch homologue (Lu).

NotchIC processing occurs only in response to binding of Notch ligands Delta or Serrate/Jagged. The post-translational modification of the nascent Notch receptor in the Golgi (Munro; Ju) appears, at least in part, to control which of the two types of ligand it interacts with on a cell surface. The Notch receptor is modified on its extracellular domain by Fringe, a glycosyl transferase enzyme that binds to the Notch/Lin motif. Fringe modifies Notch by adding O-linked fucose groups to the EGF-like repeats (Moloney; Bruckner). This modification by Fringe does not prevent ligand binding, but may influence ligand induced conformational changes in Notch. Furthermore, recent studies suggest that the action of Fringe modifies Notch to prevent it from interacting functionally with Serrate/Jagged ligands but allow it to preferentially interact with Delta (Panin; Hicks). Although Drosophila has a single Fringe gene, vertebrates are known to express multiple genes (Radical, Manic and Lunatic Fringes) (Irvine).

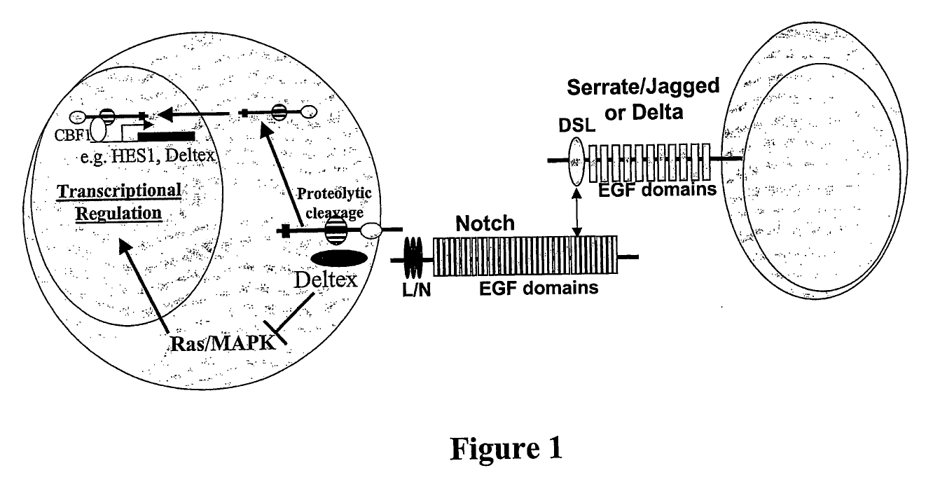

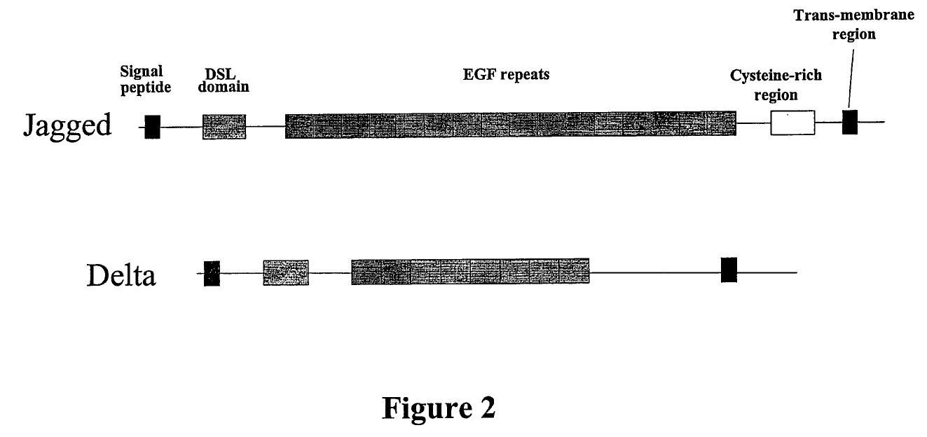

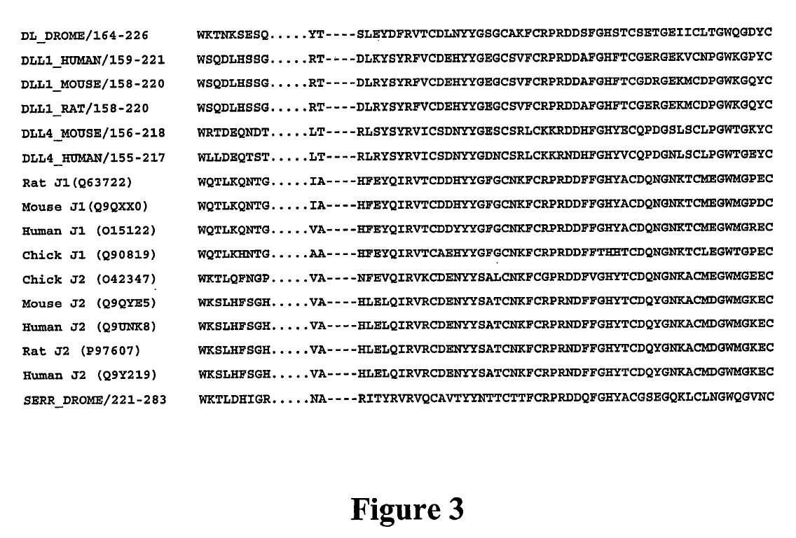

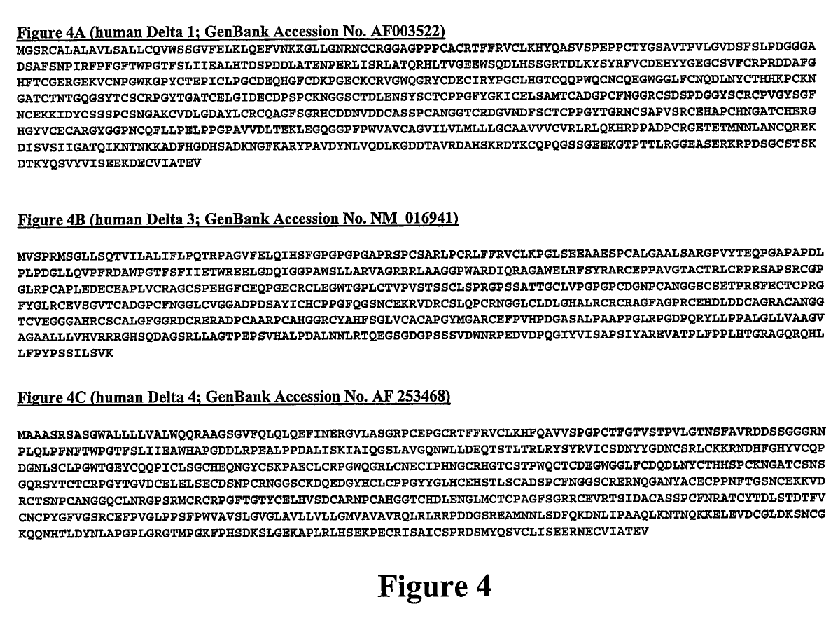

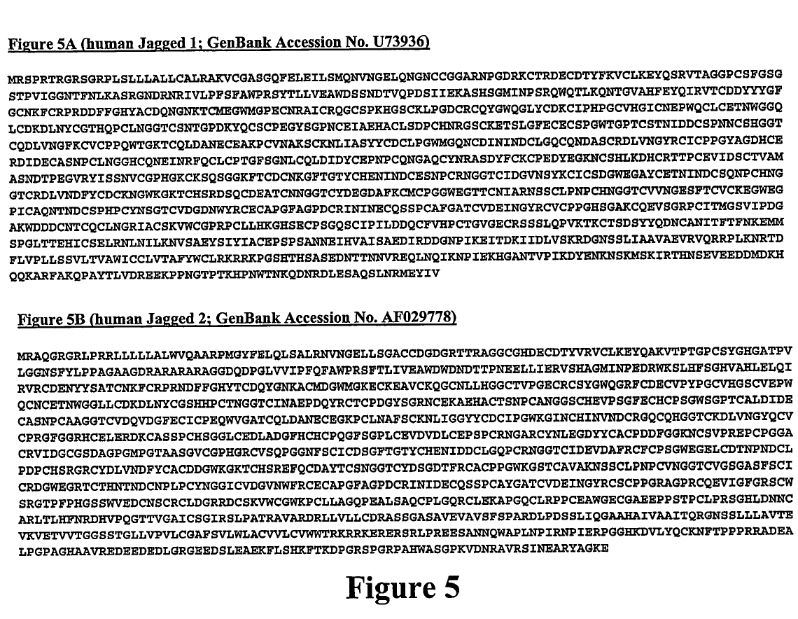

Thus, signal transduction from the Notch receptor can occur via different pathways (FIGS. 1-3). The better defined pathway involves proteolytic cleavage of the intracellular domain of Notch (NotchIC) that translocates to the nucleus and forms a transcriptional activator complex with the CSL family protein CBF1 (supressor of hairless, Su(H) in Drosophila, Lag-2 in C. elegans). NotchIC-CBF1 complexes then activate target genes, such as the bHLH proteins HES (hairy-enhancer of split like) 1 and 5. Notch can also signal in a CBF1-independent manner that involves the cytoplasmic zinc finger containing protein Deltex (FIG. 3). Unlike CBF1, Deltex does not move to the nucleus following Notch activation but instead can interact with Grb2 and modulate the Ras-Jnk signalling pathway.

As described above, several endogenous modulators of Notch are already known. These include, for example, the Notch ligands Delta and Serrate. An aim of the present invention is the detection of novel Notch signalling modulators.

The term “Notch ligand” as used herein means an agent capable of interacting with a Notch receptor to cause a biological effect. The term as used herein therefore includes naturally occurring protein ligands such as Delta and Serrate/Jagged as well as antibodies to the Notch receptor, peptidomimetics and small molecules which have corresponding biological effects to the natural ligands. Preferably the Notch ligand interacts with the Notch receptor by binding.

The term “antibody” as used herein includes intact molecules as well as fragments thereof, such as Fab, F(ab′)2, Fv and scFv which are capable of binding the epitopic determinant. These antibody fragments retain some ability to selectively bind with its antigen or receptor and include, for example:
(i) Fab, the fragment which contains a monovalent antigen-binding fragment of an antibody molecule can be produced by digestion of whole antibody with the enzyme papain to yield an intact light chain and a portion of one heavy chain;
(ii) Fab′, the fragment of an antibody molecule can be obtained by treating whole antibody with pepsin, followed by reduction, to yield an intact light chain and a portion of the heavy chain; two Fab′ fragments are obtained per antibody molecule;
(iii) F(ab′)₂, the fragment of the antibody that can be obtained by treating whole antibody with the enzyme pepsin without subsequent reduction; F(ab′)₂ is a dimer of two Fab′ fragments held together by two disulfide bonds;
(iv) scFv, including a genetically engineered fragment containing the variable region of a heavy and a light chain as a fused single chain molecule.

By a “homologue” is meant a gene product that exhibits sequence homology, either amino acid or nucleic acid sequence homology, to any one of the known Notch ligands, for example as mentioned above. Typically, a homologue of a known Notch ligand will be at least 20%, preferably at least 30%, identical at the amino acid level to the corresponding known Notch ligand over a sequence of at least 10, preferably at least 20, preferably at least 50, suitably at least 100 amino acids, or over the entire length of the Notch ligand. Techniques and software for calculating sequence homology between two or more amino acid or nucleic acid sequences are well known in the art (see for example http://www.ncbi.nlm.nih.gov and Ausubel et al., Current Protocols in Molecular Biology (1995), John Wiley & Sons, Inc.)

Homologues of Notch ligands can be identified in a number of ways, for example by probing genomic or cDNA libraries with probes comprising all or part of a nucleic acid encoding a Notch ligand under conditions of medium to high stringency (for example 0.03M sodium chloride and 0.03M sodium citrate at from about 50° C. to about 60° C.). Alternatively, homologues may also be obtained using degenerate PCR which will generally use primers designed to target sequences within the variants and homologues encoding conserved amino acid sequences. The primers will contain one or more degenerate positions and will be used at stringency conditions lower than those used for cloning sequences with single sequence primers against known sequences.

Techniques are well known in the art for the screening and development of agents such as antibodies, peptidomimetics and small organic molecules which are capable of binding to components of the Notch signalling pathway such as the Notch receptor. These include the use of phage display systems for expressing signalling proteins, and using a culture of transfected E. coli or other microorganism to produce the proteins for binding studies of potential binding compounds (see, for example, G. Cesarini, FEBS Letters, 307(1):66-70 (July 1992); H. Gram et al., J. Immunol. Meth., 161:169-176 (1993); and C. Summer et al., Proc. Natl. Acad. Sci., USA, 89:3756-3760 (May 1992)). Further library and screening techniques are described, for example, in U.S. Pat. No. 6,281,344 (Phylos).

Preferably a modulator of Notch signalling for use in the present invention will be a Notch receptor agonist such as a Notch ligand capable of binding to and activating a Notch receptor, preferably a human Notch receptor such as Notch 1, Notch2, Notch3 or Notch4. Such binding and activation may be assessed by a variety of techniques known in the art including in vitro binding assays and activity assays for example as described herein.

In the present invention Notch signalling preferably means specific signalling, meaning that the signalling results substantially or at least predominantly from the Notch signalling pathway, and preferably from Notch/Notch ligand interaction, rather than any other significant interfering or competing cause, such as cytokine signalling. Thus, in a preferred embodiment, Notch signalling excludes cytokine signalling.

Modulators of Notch Signalling

The term “modulate” as used herein refers to a change or alteration in the biological activity of the Notch signalling pathway or a target signalling pathway thereof. The term “modulator” may refer to antagonists or inhibitors of Notch signalling, i.e. compounds which block, at least to some extent, the normal biological activity of the Notch signalling pathway. Conveniently such compounds may be referred to herein as inhibitors or antagonists. Alternatively, the term “modulator” may refer to agonists of Notch signalling, i.e. compounds which stimulate or upregulate, at least to some extent, the normal biological activity of the Notch signalling pathway. Conveniently such compounds may be referred to as upregulators or agonists.

The term “candidate modulator” is used to describe any one or more molecule(s) which may be, or is suspected of being, capable of functioning as a modulator of Notch signalling. Said molecules may for example be organic “small molecules” or polypeptides. Suitably, candidate molecules comprise a plurality of, or a library of such molecules or polypeptides. These molecules may be derived from known modulators. “Derived from” means that the candidate modulator molecules preferably comprise polypeptides which have been fully or partially randomised from a starting sequence which is a known modulator of Notch signalling. Most preferably, candidate molecules comprise polypeptides which are at least 40% homologous, more preferably at least 60% homologous, even more preferably at least 75% homologous or even more, for example 85%, or 90%, or even more than 95% homologous to one or more known Notch modulator molecules, using the BLAST algorithm with the parameters as defined herein.

The candidate modulator of the present invention may be an organic compound or other chemical. In this embodiment, the candidate modulator will be an organic compound comprising two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. The candidate modulator may comprise at least one cyclic group. The cyclic group may be a polycyclic group, such as a non-fused polycyclic group. For some applications, the agent comprises at least the one of said cyclic groups linked to another hydrocarbyl group.

In one preferred embodiment, the candidate compound will be an amino acid sequence or a chemical derivative thereof, or a combination thereof. In another preferred embodiment, the candidate compound will be a nucleotide sequence, which may be a sense sequence or an anti-sense sequence. The candidate modulator may also be an antibody.

Candidate modulators may be synthetic compounds or natural isolated compounds. Various examples of such synthetic or natural modulators are listed below.

Agonists of Notch signalling will include any molecule which is capable of up-regulating Notch, the Notch signalling pathway or any one or more of the components of the Notch signalling pathway. Candidate modulators for up-regulating the Notch signalling pathway include compounds capable of transducing or activating the Notch signalling pathway.

Modulators for Notch signalling transduction will include molecules which participate in signalling through Notch receptors including activation of Notch, the downstream events of the Notch signalling pathway, transcriptional regulation of downstream target genes and other non-transcriptional downstream events (e.g. post-translational modification of existing proteins). More particularly, such modulators will allow activation of target genes of the Notch signalling pathway.

According to one aspect of the present invention the modulator may be the Notch polypeptide or polynucleotide or a fragment, variant, derivative, mimetic or homologue thereof which retains the signalling transduction ability of Notch or an analogue of Notch which has the signalling transduction ability of Notch. By Notch, we mean Notch-1, Notch-2, Notch-3, Notch-4 and any other Notch homologues or analogues. Analogues of Notch include proteins from the Epstein Barr virus (EBV), such as EBNA2, BARF0 or LMP2A. In a particularly preferred embodiment the modulator may be the Notch intracellular domain (Notch IC) or a sub-fragment, variant, derivative, mimetic, analogue or homologue thereof.

Modulators for Notch signalling activation include molecules which are capable of activating Notch, the Notch signalling pathway or any one or more of the components of the Notch signalling pathway.

Such a modulator may be a dominant negative version of a Notch signalling repressor. In an alternative embodiment, the modulator will be capable of inhibiting a Notch signalling repressor. In a further alternative embodiment, the modulator for Notch signalling activation will be a positive activator of Notch signalling.

In a particular embodiment, the modulator will be capable of inducing or increasing Notch or Notch ligand expression. Such a molecule may be a nucleic acid sequence capable of inducing or increasing Notch or Notch ligand expression.

In one embodiment, the modulator will be capable of up-regulating expression of the endogenous genes encoding Notch or Notch ligands in target cells. In particular, the modulator may be an immunosuppressive cytokine capable of up-regulating the expression of endogenous Notch or Notch ligands in target cells, or a polynucleotide which encodes such a cytokine. Immunosuppressive cytokines include IL-10, IL-13, TGF-beta and FLT3 ligand. Candidate modulators will therefore further include fragments, derivatives, variants, mimetics, analogues and homologues of any of the above.

Endogenous agonists include Noggin, Chordin, Follistatin, Xnr3, fibroblast growth factors. Candidate modulators may therefore include derivatives, fragments, variants, mimetics, analogues and homologues thereof, or a polynucleotide encoding any one or more of the above.

In another embodiment, the modulator may be a Notch ligand, or a polynucleotide encoding a Notch ligand. Notch ligands will typically be capable of binding to a Notch receptor polypeptide present in the membrane of a variety of mammalian cells, for example hemapoietic stem cells. Endogenous Notch ligands include polypeptides of the Delta family, for example Delta-1 (Genbank Accession No. AF003522—Homo sapiens), Delta-3 (Genbank Accession No. AF084576—Rattus norvegicus), Delta-like 3 (Mus musculus), Delta-4 (Genbank Accession No. AB043894) and polypeptides of the Serrate family, for example Serrate-1 and Serrate-2 (WO97/01571, WO96/27610 and WO92/19734), Jagged-1 and Jagged-2 (Genbank Accession No. AF029778—Homo sapiens), and LAG-2. Candidate compounds of the present invention include fragments, derivatives, variants, mimetics, analogues and homologues of any of the above.

In a preferred embodiment, the modulator will be a constitutively active Notch receptor or Notch intracellular domain, or a polynucleotide encoding such a receptor or intracellular domain.

In an alternative embodiment, the modulator of Notch signalling will act downstream of the Notch receptor. Thus, for example, the activator of Notch signalling may be a constitutively active Deltex polypeptide or a polynucleotide encoding such a polypeptide. Other endogenous downstream components of the Notch signalling pathway include Deltex-1, Deltex-2, Deltex-3, Suppressor of Deltex (SuDx), Numb and isoforms thereof, Numb associated Kinase (NAK), Notchless, Dishevelled (Dsh), emb5, Fringe genes (such as Radical, Lunatic and Manic), PON, LNX, Disabled, Numblike, Nur77, NFkB2, Mirror, Warthog, Engrailed-1 and Engrailed-2, Lip-1 and homologues thereof, the polypeptides involved in the Ras/MAPK cascade modulated by Deltex, polypeptides involved in the proteolytic cleavage of Notch such as Presenilin and polypeptides involved in the transcriptional regulation of Notch target genes. Candidate modulators of use in the present invention will therefore include constitutively active forms of any of the above, analogues, homologues, derivatives, variants, mimetics and fragments thereof.

Modulators for Notch signalling activation may also include any polypeptides expressed as a result of Notch activation and any polypeptides involved in the expression of such polypeptides, or polynucleotides encoding for such polypeptides.

Activation of Notch signalling may also be achieved by repressing inhibitors of the Notch signalling pathway. As such, candidate modulators will include molecules capable of repressing any Notch signalling inhibitors. Preferably the molecule will be a polypeptide, or a polynucleotide encoding such a polypeptide, that decreases or interferes with the production or activity of compounds that are capable of producing an decrease in the expression or activity of Notch, Notch ligands, or any downstream components of the Notch signalling pathway. In a preferred embodiment, the modulators will be capable of repressing polypeptides of the Toll-like receptor protein family, cytokines such as IL-12, IFN-γ, TNF-α, and growth factors such as the bone morphogenetic protein (BMP), BMP receptors and activins.

Preferably, the modulator of the present invention will be a polypeptide or a polynucleotide.

Whether or not any given agent acts as a modulator of Notch signalling (and if so whether it is an activator or inhibitor of such signalling) may be readily determined by use of suitable assays or screens, for example, those described in the Examples herein.

Notch Ligand Domains

As discussed above, Notch ligands typically comprise a number of distinctive domains. Some predicted/potential domain locations for various naturally occurring human Notch ligands (based on amino acid numbering in the precursor proteins) are shown below:

Human Delta 1

| Component | Amino acids | Proposed function/domain |
|---|---|---|
| SIGNAL | 1-17 | SIGNAL |
| CHAIN | 18-723 | DELTA-LIKE PROTEIN 1 |
| DOMAIN | 18-545 | EXTRACELLULAR |
| TRANSMEM | 546-568 | TRANSMEMBRANE |
| DOMAIN | 569-723 | CYTOPLASMIC |
| DOMAIN | 159-221 | DSL |
| DOMAIN | 226-254 | EGF-LIKE 1 |
| DOMAIN | 257-285 | EGF-LIKE 2 |
| DOMAIN | 292-325 | EGF-LIKE 3 |
| DOMAIN | 332-363 | EGF-LIKE 4 |
| DOMAIN | 370-402 | EGF-LIKE 5 |
| DOMAIN | 409-440 | EGF-LIKE 6 |
| DOMAIN | 447-478 | EGF-LIKE 7 |
| DOMAIN | 485-516 | EGF-LIKE 8 |

Human Delta 3

| Component | Amino acids | Proposed function/domain |
|---|---|---|
| DOMAIN | 158-248 | DSL |
| DOMAIN | 278-309 | EGF-LIKE 1 |
| DOMAIN | 316-350 | EGF-LIKE 2 |
| DOMAIN | 357-388 | EGF-LIKE 3 |
| DOMAIN | 395-426 | EGF-LIKE 4 |
| DOMAIN | 433-464 | EGF-LIKE 5 |

Human Delta 4

| Component | Amino acids | Proposed function/domain |
|---|---|---|
| SIGNAL | 1-26 | SIGNAL |
| CHAIN | 27-685 | DELTA-LIKE PROTEIN 4 |
| DOMAIN | 27-529 | EXTRACELLULAR |
| TRANSMEM | 530-550 | TRANSMEMBRANE |
| DOMAIN | 551-685 | CYTOPLASMIC |
| DOMAIN | 155-217 | DSL |
| DOMAIN | 218-251 | EGF-LIKE 1 |
| DOMAIN | 252-282 | EGF-LIKE 2 |
| DOMAIN | 284-322 | EGF-LIKE 3 |
| DOMAIN | 324-360 | EGF-LIKE 4 |
| DOMAIN | 362-400 | EGF-LIKE 5 |
| DOMAIN | 402-438 | EGF-LIKE 6 |
| DOMAIN | 440-476 | EGF-LIKE 7 |
| DOMAIN | 480-518 | EGF-LIKE 8 |

Human Jagged 1

| Component | Amino acids | Proposed function/domain |
|---|---|---|
| SIGNAL | 1-33 | SIGNAL |
| CHAIN | 34-1218 | JAGGED 1 |
| DOMAIN | 34-1067 | EXTRACELLULAR |
| TRANSMEM | 1068-1093 | TRANSMEMBRANE |
| DOMAIN | 1094-1218 | CYTOPLASMIC |
| DOMAIN | 167-229 | DSL |
| DOMAIN | 234-262 | EGF-LIKE 1 |
| DOMAIN | 265-293 | EGF-LIKE 2 |
| DOMAIN | 300-333 | EGF-LIKE 3 |
| DOMAIN | 340-371 | EGF-LIKE 4 |
| DOMAIN | 378-409 | EGF-LIKE 5 |
| DOMAIN | 416-447 | EGF-LIKE 6 |
| DOMAIN | 454-484 | EGF-LIKE 7 |
| DOMAIN | 491-522 | EGF-LIKE 8 |
| DOMAIN | 529-560 | EGF-LIKE 9 |
| DOMAIN | 595-626 | EGF-LIKE 10 |
| DOMAIN | 633-664 | EGF-LIKE 11 |
| DOMAIN | 671-702 | EGF-LIKE 12 |
| DOMAIN | 709-740 | EGF-LIKE 13 |
| DOMAIN | 748-779 | EGF-LIKE 14 |
| DOMAIN | 786-817 | EGF-LIKE 15 |
| DOMAIN | 824-855 | EGF-LIKE 16 |
| DOMAIN | 863-917 | VON WILLEBRAND FACTOR C |

Human Jagged 2

| Component | Amino acids | Proposed function/domain |
|---|---|---|
| SIGNAL | 1-26 | SIGNAL |
| CHAIN | 27-1238 | JAGGED 2 |
| DOMAIN | 27-1080 | EXTRACELLULAR |
| TRANSMEM | 1081-1105 | TRANSMEMBRANE |
| DOMAIN | 1106-1238 | CYTOPLASMIC |
| DOMAIN | 178-240 | DSL |
| DOMAIN | 249-273 | EGF-LIKE 1 |
| DOMAIN | 276-304 | EGF-LIKE 2 |
| DOMAIN | 311-344 | EGF-LIKE 3 |
| DOMAIN | 351-382 | EGF-LIKE 4 |
| DOMAIN | 389-420 | EGF-LIKE 5 |
| DOMAIN | 427-458 | EGF-LIKE 6 |
| DOMAIN | 465-495 | EGF-LIKE 7 |
| DOMAIN | 502-533 | EGF-LIKE 8 |
| DOMAIN | 540-571 | EGF-LIKE 9 |
| DOMAIN | 602-633 | EGF-LIKE 10 |
| DOMAIN | 640-671 | EGF-LIKE 11 |
| DOMAIN | 678-709 | EGF-LIKE 12 |
| DOMAIN | 716-747 | EGF-LIKE 13 |
| DOMAIN | 755-786 | EGF-LIKE 14 |
| DOMAIN | 793-824 | EGF-LIKE 15 |
| DOMAIN | 831-862 | EGF-LIKE 16 |
| DOMAIN | 872-949 | VON WILLEBRAND FACTOR C |

DSL Domain

A typical DSL domain may include most or all of the following consensus amino acid sequence (SEQ ID NO:23):

Cys Xaa Xaa Xaa Xaa Xaa Xaa Xaa Xaa Cys Xaa Xaa
Xaa Cys Xaa Xaa
Xaa Xaa Xaa Xaa Xaa Xaa Xaa Xaa Xaa Cys Xaa Xaa
Xaa Xaa Xaa Xaa
Xaa Cys Xaa Xaa Xaa Xaa Xaa Xaa Xaa Xaa Cys

Preferably the DSL domain may include most or all of the following consensus amino acid sequence (SEQ ID NO:24):

Cys Xaa Xaa Xaa ARO ARO Xaa Xaa Xaa Cys Xaa Xaa
Xaa Cys BAS NOP
BAS ACM ACM Xaa ARO NOP ARO Xaa Xaa Cys Xaa Xaa
Xaa NOP Xaa Xaa
Xaa Cys Xaa Xaa NOP ARO Xaa NOP Xaa Xaa Cys

wherein:
ARO is an aromatic amino acid residue, such as tyrosine, phenylalanine, tryptophan or histidine;
NOP is a non-polar amino acid residue such as glycine, alanine, proline, leucine, isoleucine or valine;
BAS is a basic amino acid residue such as arginine or lysine; and
ACM is an acid or amide amino acid residue such as aspartic acid, glutamic acid, asparagine or glutamine.

Preferably the DSL domain may include most or all of the following consensus amino acid sequence (SEQ ID NO:25):

Cys Xaa Xaa Xaa Tyr Tyr Xaa Xaa Xaa Cys Xaa Xaa
Xaa Cys Arg Pro
Arg Asx Asp Xaa Phe Gly His Xaa Xaa Cys Xaa Xaa
Xaa Gly Xaa Xaa
Xaa Cys Xaa Xaa Gly Trp Xaa Gly Xaa Xaa Cys

(wherein Xaa may be any amino acid and Asx is either aspartic acid or asparagine).

An alignment of DSL domains from Notch ligands from various sources is shown in FIG. 3.

The DSL domain used may be derived from any suitable species, including for example Drosophila, Xenopus, rat, mouse or human. Preferably the DSL domain is derived from a vertebrate, preferably a mammalian, preferably a human Notch ligand sequence.

Suitably, for example, a DSL domain for use in the present invention may have at least 30%, preferably at least 50%, preferably at least 60%, preferably at least 70%, preferably at least 80%, preferably at least 90%, preferably at least 95% amino acid sequence identity to the DSL domain of human Jagged 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Jagged 2.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 1.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 3.

Alternatively a DSL domain for use in the present invention may, for example, have at least 30%, preferably at least 50%, preferably at least 60%, preferably at least 70%, preferably at least 80%, preferably at least 90%, preferably at least 95% amino acid sequence identity to the DSL domain of human Delta 4.

The term “Notch ligand N-terminal domain” means the part of a Notch ligand sequence from the N-terminus to the start of the DSL domain. It will be appreciated that this term includes sequence variants, fragments, derivatives and mimetics having activity corresponding to naturally occurring domains.

The term “heterologous amino acid sequence” or “heterologous nucleotide sequence” as used herein means a sequence which is not found in the native sequence (e.g. in the case of a Notch ligand sequence is not found in the native Notch ligand sequence) or its coding sequence. Preferably any such heterologous amino acid sequence is not a TSST sequence, and preferably it is not a superantigen sequence.

EGF-Like Domain

The EGF-like motif has been found in a variety of proteins, as well as EGF and Notch and Notch ligands, including those involved in the blood clotting cascade (Furie and Furie, 1988, Cell 53: 505-518). For example, this motif has been found in extracellular proteins such as the blood clotting factors IX and X (Rees et al., 1988, EMBO J. 7:2053-2061; Furie and Furie, 1988, Cell 53: 505-518), in other Drosophila genes (Knust et al., 1987 EMBO J. 761-766; Rothberg et al., 1988, Cell 55:1047-1059), and in some cell-surface receptor proteins, such as thrombomodulin (Suzuki et al., 1987, EMBO J. 6:1891-1897) and LDL receptor (Sudhof et al., 1985, Science 228:815-822). A protein binding site has been mapped to the EGF repeat domain in thrombomodulin and urokinase (Kurosawa et al., 1988, J. Biol. Chem 263:5993-5996; Appella et al., 1987, J. Biol. Chem. 262:4437-4440).

As reported by PROSITE a typical EGF-like domain may include six cysteine residues which have been shown (in EGF) to be involved in disulfide bonds. The main structure is proposed, but not necessarily required, to be a two-stranded beta-sheet followed by a loop to a C-terminal short two-stranded sheet. Subdomains between the conserved cysteines strongly vary in length as shown in the following schematic representation of a typical EGF-like domain (SEQ ID NO:26):

```
                              +−−−−−−−−−−−−−−−−−−−+ +−−−−−−−−−−−−−−−−−−−−−−−−−+
                              | | | |
x(4)-C-x(0,48)-C-x(3,12)-C-x(1,70)-C-x(1,6)-C-x(2)-G-a-x(0,21)-G-x(2)-C-x
          | | ************************************
          +−−−−−−−−−−−−−−−−−−−+
```

wherein:
‘C’: conserved cysteine involved in a disulfide bond.
‘G’: often conserved glycine
‘a’: often conserved aromatic amino acid
‘*’: position of both patterns.
‘x’: any residue

The region between the 5th and 6th cysteine contains two conserved glycines of which at least one is normally present in most EGF-like domains.

The EGF-like domain used may be derived from any suitable species, including for example Drosophila, Xenopus, rat, mouse or human. Preferably the EGF-like domain is derived from a vertebrate, preferably a mammalian, preferably a human Notch ligand sequence.

Suitably, for example, an EGF-like domain for use in the present invention may have at least 30%, preferably at least 50%, preferably at least 60%, preferably at least 70%, preferably at least 80%, preferably at least 90%, preferably at least 95% amino acid sequence identity to an EGF-like domain of human Jagged 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Jagged 2.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 1.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 3.

Alternatively an EGF-like domain for use in the present invention may, for example, have at least 30%, preferably at least 50%, preferably at least 60%, preferably at least 70%, preferably at least 80%, preferably at least 90%, preferably at least 95% amino acid sequence identity to an EGF-like domain of human Delta 4.

Antibodies

In one embodiment the modulator of Notch signalling may be an antibody, derivative or fragment which binds to and activates Notch. Thus the invention also provides a support (e.g. bead, plate or well, preferably a bead) to which is coupled (e.g., chemically, by affinity or adsportion) an antibody capable of binding to and activating Notch.

General methods of making antibodies are known in the art. (See for example, Harlow and Lane, Antibodies: A Laboratory Manual, Cold Spring Harbor Laboratory, New York (1988), the text of which is incorporated herein by reference). Antibodies may be monoclonal or polyclonal but are preferably monoclonal.

Suitably, the binding affinity (equilibrium association constant (Ka)) may be at least about 10⁶ M⁻¹, at least about 10⁷ M⁻¹, at least about 10⁸ M⁻¹ or at least about 10⁹ M⁻¹.

Suitably the antibody, derivative or fragment binds to one or more EGF or Lin/Notch (L/N) domains of Notch (for example to EGF repeats 11 and 12 of Notch).

Suitable antibodies for use as blocking agents are obtained by immunizing a host animal with peptides comprising all or a portion of Notch.

The peptide used may comprise the complete protein or a fragment or derivatives thereof. Preferred immunogens comprise all or a part of the extracellular domain of human Notch (e.g., Notch1, Notch2, Notch3 or Notch4, preferably Notch1 or Notch2), where these residues contain any post-translation modifications, such as glycosylation, found in the native proteins. Immunogens comprising the extracellular domain may be produced by a number of techniques which are well known in the art such as expression of cloned genes using conventional recombinant methods and/or isolation from T cells or cell populations expressing high levels of Notch.

Monoclonal antibodies may be produced by means well known in the art. Generally, the spleen and/or lymph nodes of an immunized host animal provide a source of plasma cells. The plasma cells are immortalized by fusion with myeloma cells to produce hybridoma cells. Culture supernatant from individual hybridomas is screened using standard techniques to identify those producing antibodies with the desired specificity. The antibody may be purified from the hybridoma cell supernatants or ascites fluid by conventional techniques, such as affinity chromatography using Notch, Notch ligands or fragments thereof bound to an insoluble support, protein A sepharose, or the like.

For example, antibodies against Notch are described in U.S. Pat. No. 5,648,464, U.S. Pat. No. 5,849,869 and U.S. Pat. No. 6,004,924 (Yale University/Imperial Cancer Technology), the texts of which are herein incorporated by reference.

Antibodies generated against the Notch receptor are also described in WO 0020576 (the text of which is also incorporated herein by reference). For example, this document discloses generation of antibodies against the human Notch-1 EGF-like repeats 11 and 12. For example, in particular embodiments, WO 0020576 discloses a monoclonal antibody secreted by a hybridoma designated A6 having the ATCC Accession No. HB12654, a monoclonal antibody secreted by a hybridoma designated Cll having the ATCC Accession No. HB12656 and a monoclonal antibody secreted by a hybridoma designated F3 having the ATCC Accession No. HB12655.

Suitably, antibodies for use to treat human patients in vivo will be chimeric or humanised antibodies. Antibody “humanisation” techniques are well known in the art. These techniques typically involve the use of recombinant DNA technology to manipulate DNA sequences encoding the polypeptide chains of the antibody molecule.

As described in U.S. Pat. No. 5,859,205 early methods for humanising monoclonal antibodies (Mabs) involved production of chimeric antibodies in which an antigen binding site comprising the complete variable domains of one antibody is linked to constant domains derived from another antibody. Such chimerisation procedures are described in EP-A-0120694 (Celltech Limited), EP-A-0125023 (Genentech Inc. and City of Hope), EP-A-0171496 (Res. Dev. Corp. Japan), EP-A-0 173 494 (Stanford University), and WO 86/01533 (Celltech Limited). For example, WO 86/01533 discloses a process for preparing an antibody molecule having the variable domains from a mouse MAb and the constant domains from a human immunoglobulin.

In an alternative approach, described in EP-A-0239400 (Winter), the complementarity determining regions (CDRs) of a mouse MAb are grafted onto the framework regions of the variable domains of a human immunoglobulin by site directed mutagenesis using long oligonucleotides. Such CDR-grafted humanised antibodies are much less likely to give rise to an anti-antibody response than humanised chimeric antibodies in view of the much lower proportion of non-human amino acid sequence which they contain. Examples in which a mouse MAb recognising lysozyme and a rat MAb recognising an antigen on human T-cells were humanised by CDR-grafting have been described by Verhoeyen et al (Science, 239, 1534-1536, 1988) and Riechmann et al (Nature, 332, 323-324, 1988) respectively. The preparation of CDR-grafted antibody to the antigen on human T cells is also described in WO 89/07452 (Medical Research Council).

In WO 90/07861 Queen et al propose four criteria for designing humanised immunoglobulins. The first criterion is to use as the human acceptor the framework from a particular human immunoglobulin that is unusually homologous to the non-human donor immunoglobulin to be humanised, or to use a consensus framework from many human antibodies. The second criterion is to use the donor amino acid rather than the acceptor if the human acceptor residue is unusual and the donor residue is typical for human sequences at a specific residue of the framework. The third criterion is to use the donor framework amino acid residue rather than the acceptor at positions immediately adjacent to the CDRs. The fourth criterion is to use the donor amino acid residue at framework positions at which the amino acid is predicted to have a side chain atom within about 3 A of the CDRs in a three-dimensional immunoglobulin model and to be capable of interacting with the antigen or with the CDRs of the humanised immunoglobulin. It is proposed that criteria two, three or four may be applied in addition or alternatively to criterion one, and may be applied singly or in any combination.

The choice of isotype will be guided by the desired effector functions, such as complement fixation, or activity in antibody-dependent cellular cytotoxicity. Suitable isotypes include IgG 1, IgG3 and IgG4. Suitably, either of the human light chain constant regions, kappa or lambda, may be used.

Cross-Linking

In one embodiment, the invention may utilise a construct comprising a multiplicity of modulators of Notch signalling in cross-linked form.

In such an embodiment, the modulators of Notch signalling may, for example, be linked to each other directly or indirectly. Linkages may be covalent or non-covalent (e.g., via electrostatic and/or hydrophobic interactions).

In one embodiment, direct linkage between modulators of Notch signalling is achieved by chemical cross-linking. Suitable chemical cross-linking procedures are well-known in the art; see, for example, Carlsson J. et al., Biochem. J. 173:723-737, 1978; Cumber, J. A. et al. Methods in Enzymology 112:207-224, 1985; Walden, P. et al., J. Mol. Cell Immunol. 2:191-197, 1986; Gordon, R. D. et al., Proc. Natl. Acad. Sci. (USA) 84:308-312, 1987; Avrameas, S. et al., Immuno-chemistry 6:53, 1969; Joseph, K. C. et al., Proc. Natl. Acad. Sci. USA 75:2815-2819, 1978; Middlebrook, J. L. et al., Academic Press, New York, pp. 311-350, 1981).

In an alternative embodiment, direct linkage may be achieved by the design and expression of a recombinant chimeric gene encoding a multiplicity of modulators of Notch signalling. The chimeric gene is then expressed in a suitable expression system.

Indirect linkage between modulators of Notch signalling may be achieved using a spacer molecule. For example, in one embodiment, the spacer may comprise an antibody, which may, for example, be a monofunctional or bifunctional antibody or antibody derivative.

Substrate Binding

Alternatively or in addition, the invention may utilise a construct comprising a multiplicity of modulators of Notch signalling bound to a substrate.

It will be appreciated that the substrate can take many different forms such as polymers, plastics, porous materials such as resin or modified cellulose, beads (such as microspheres and microbeads, nanospheres and nanoparticles), laboratory plates and wells and liposomes.

For example, for ex-vivo uses, the support may be a plate such as a microtiter plate, or for example a well or other suitable container. At least part of the surface may be coated with modulators of Notch signalling bound by covalent or non-covalent means.

In one embodiment the substrate may be a particulate substrate, such as a bead, sphere, particle or carrier, for example having a diameter (or, for example, within a collection of beads, a mean diameter) of from about 0.001 to about 1000 micrometres, for example from about 0.01 to about 100 micrometres, suitably from about 0.1 to 10 micrometres, for example about 1 to 10 micrometres. Particulate materials such as beads have the advantages of being easier to handle in certain situations, and of potentially providing a larger surface area for interaction with cells. They may also be more suitable for in-vivo applications, especially when the substrate comprises a biodegradable material.

It will be appreciated that the term “diameter” normally applies to particles having a substantially spherical or other circular form. However, it will be appreciated that particles used in the present invention do not need to have such a regular form, and may have a more irregular form, in which case the relevant dimension is suitably the largest linear dimension.

In addition, where dimensions are given for individual particles or beads, it will be appreciated that these apply also to collections or populations of particles or beads, in which case the dimension given will relate to the average dimension of the collection or population, suitably the mean dimension. For example, where it is stated that a bead has a diameter in a given range, it will be appreciated that this can also be considered in terms of a collection or population of beads having a mean diameter in the same range.

A preferred particle or bead size is from 20-1000 nm, for example about 100 nm (0.1 microns).

Substrates may, for example, comprise natural or synthetic polymers such as polystyrene, polyethylene glycol (PEG), polyglycollic acid (PGA), polycaprolactide, polyacrylamide, latex, silica, dextran, agarose, starch, cellulose, chitin/chitosan, polylactide, poly(methylmethacrylate) (PMMA); proteins/polypeptides such as albumins, for example human serum albumins; and modified, crosslinked and derivatized embodiments thereof. Suitable materials include, for example polystyrene, cellulose, dextran crosslinked with epichlorohydrin (Sephadex.™., Pharmacia, Uppsala, Sweden), polyacrylamide crosslinked with bisacrylamide (Biogel.™., BioRad, USA), agar, glass beads, polylactide beads and latex beads. Derivatized microparticles include microparticles derivatized with, e.g. maleimide, aldehyde groups, allyl groups, carboxyalkyl groups such as carboxymethyl, phosphoryl and substituted phosphoryl groups, sulfate, sulfhydryl and sulfonyl groups, and amino and substituted amino groups. Beads may have either hydrophilic or hydrophobic properties.

The modulators of Notch signalling may be bound to the substrate (e.g. bead) by any suitable means. For example, binding may be by non-covalent linking such as surface adsorption (e.g. by hydrophobic and/or electrostatic interactions) or by covalent linking such as chemical linking. For example, reactive groups (such as amino, aldehyde, carboxy, epoxy or toluenesulfonyl (tosyl), thiol or maleimide groups) may be present on or introduced onto a substrate (such as a bead) surface and these may be linked to modulators of Notch signalling.

For example, modulators of Notch signalling in the form of proteins or polypeptides may be linked to a substrate (such as a bead) by incubation of a surface activated substrate (such as a bead) with the protein or polypeptide, suitably by incubation for at least 12 to 24 hours suitably at neutral or neutral to high pH and suitably at a sufficiently high temperature such that a reactive group on the substrate/bead (for example a tosyl, epoxy, amino, carboxy, aldehyde, thiol or maleimide group) reacts with a reactive group on the protein or polypeptide (for example a free amino or sulfhydryl group on the protein or polypeptide, or an aldehyde group) to form a covalent link.

A variety of linker groups may also be used to bind the substrate/particle/bead to the modulators of Notch signalling if required. Suitable linkers are well known in the art and suitably comprise an acid, basic, aldehyde, ether or ester reactive group or a residue thereof. Suitable linker moieties include, for example, succinimidyl propionate, succinimidyl butanoate, N-hydroxysuccinimide, benzotriazole carbonate, propionaldehyde, maleimide or forked maleimide, biotin, vinyl derivative and phospholipids.

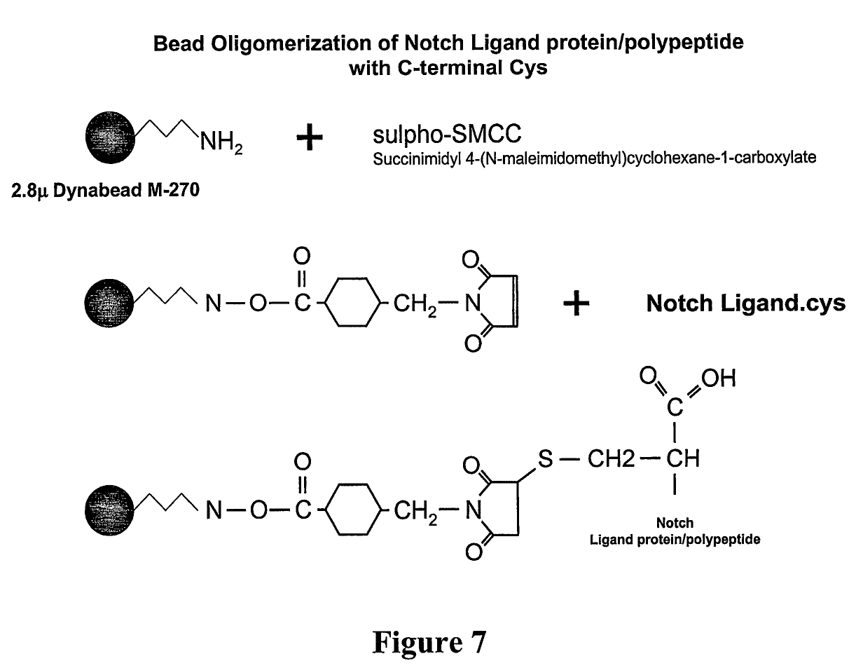

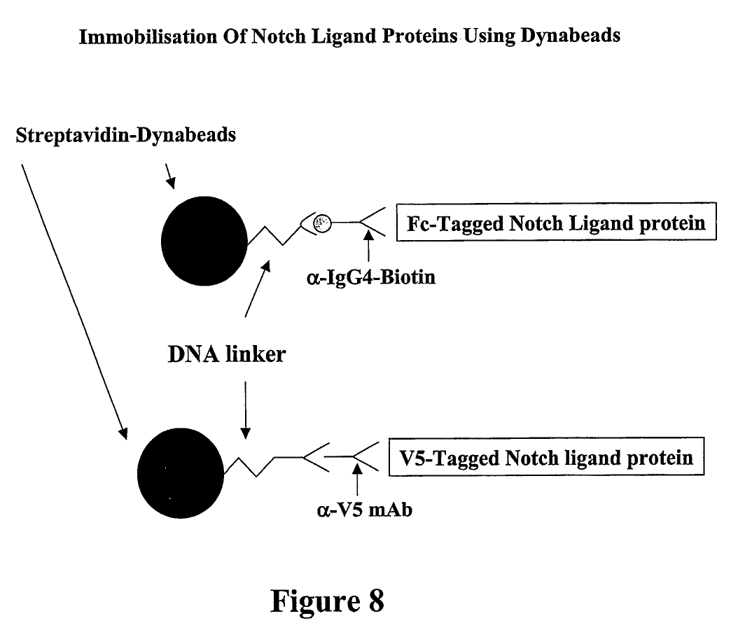

For example, modulators of Notch signalling such as Notch ligand proteins and polypeptides may be chemically coupled to beads using a coupling agent such as sulpho-SMCC (succinimidyl 4-(N-maleimidomethyl)cyclohexane-1-carboxylate). A reaction scheme is shown in FIG. 7.

Antibodies may also be used to bind modulators of Notch signalling to a substrate (such as a bead). For example, in one of many embodiments, beads coated with antibody-binding materials such as streptavidin may be used to bind a biotinylated anti-IgG antibody, which in turn may be bound to a modulator of Notch signalling in the form of an IgG fusion protein. Alternatively, for example, suitably functionalised agents such as biotinylated agents/ligands may be directly coupled to beads such as streptavidin beads.

In one embodiment, particles or beads may be magnetic particles such as magnetic beads, such as for example those available under the trade name DYNABEAD™ from Dynal Biotech, Oslo, Norway. These have the advantage of being easy to separate from fluids magnetically, which can be particularly advantageous for use ex-vivo uses. Such particles or beads may be prepared, for example, by incorporating a magnetic or paramagnetic material such as iron oxide into a polymeric matrix such as polystyrene.

As described on the Dynal Biotech web-site (www.dynal.no), Dynabeads™ are superparamagnetic polymer spheres, magnetic only when placed in a magnetic field and with no residual magnetism when the magnetic field is removed. They are composed of highly cross-linked polystyrene with magnetic material precipitated in pores evenly distributed throughout each bead. The pores are preferably filled with an additional polymer layer, which seals the iron material inside the beads. Chemical groups (such as amino, carboxy, epoxy or toluenesulfonyl (tosyl) groups) may then be introduced on the bead surface if appropriate.

Particles or beads may be either hydrophilic or hydrophobic. Hydrophilic particles or beads (such as, for example, Dynabeads available from Dynal under the serial number M270) allow gentle coupling of ligands to the surface to maintain functional activity of labile proteins. The hydrophilic properties of the beads ensure optimal dispersity in aqueous solution. These particles or beads may be surface activated with, for example amine and carboxylic acid functional groups. The functional groups of the amine and carboxylic acid beads allow further introduction of a large variety of alternative reactive groups by coupling to commercially available cross-linkers.

Hydrophobic particles or beads (such as, for example, Dynabeads available from Dynal under the serial numbers M-450 and M-280) are well suited to coupling of antibodies. The hydrophobic Fc region of the antibody may be adsorbed to the hydrophobic particle or bead surface, followed by a rapid covalent bond formation. The orientation of the antibody is thereby generally optimal with the Fab regions facing outwards.

It will be appreciated that the orientation of the active site of the ligand or compound to be coupled to the matrix, particles or beads may also need to be taken into consideration. Hydrophobic particles or beads facilitate hydrophobic-hydrophobic interactions between the particles or beads and the protein's hydrophobic parts, whereas the hydrophilic beads are suited when hydrophilic-hydrophilic interactions between the particles or beads and the protein's hydrophilic parts are desired.

In one embodiment, the particles or beads may for example be latex microspheres such as those available from Interfacial Dynamics Corporation (Portland, US). As disclosed on the Interfacial Dynamics web-site (www.idclatex.com) latex microspheres/beads are available with either anionic (negative) or cationic (positive) surface charges. Anionic latexes—such as those with sulfate, carboxyl, or carboxylate modified surface groups—are less likely to bind to negatively-charged cell surfaces and are therefore used frequently in biological applications.

Particles or beads may suitably be sterilized before use, for example by pasteurization, suitably for about 24 hours at 78-80° C.; or by gamma irradiation, for example at 0.03 megarads suitably for about 24 hours.

Suitably the particles or beads may be coated with various proteins or polysaccharides that will greatly reduce their capacity to absorb biomolecules non-specifically. Specific irreversible adsorption of protein molecules such as avidin, streptavidin, and antibodies may be accomplished by simply mixing the latex and protein together for a specified period of time, then separating the bound from the unbound protein through centrifugation and removal of the supernatant.

To reduce nonspecific binding, particles or beads may be coated, for example with BSA or dextrans. To further reduce nonspecific binding, proteins, nucleic acids, and other biomolecules may if desired be covalently coupled to the particles or beads. Covalent coupling may require more effort than passive adsorption, but can result in conjugates with greater specificity that remain active longer. Carbodiimide-mediated coupling to CML latexes is a suitable method for conjugating low molecular weight peptides and oligonucleotides.

A wide range of suitable beads, micro/nanobeads and micro/nanospheres is also available for example from Polysciences, Inc. 400 Valley Road, Warrington, Pa., US.

In an alternative embodiment, modulators of Notch signalling may be conjugated to the linear or cross-linked backbone of a liposome using conventional techniques (see, e.g. Ostro, M. J. (Ed.), Liposomes: from Biophysics to Therapeutics (Marcel Dekker, New York, 1987)). One preferred method of preparing liposomes and conjugating immunoglobulins to their surface is described by Ishimoto, Y. et al., J. Immunol. Met. 75, 351-360 (1984). For example, multilamillar liposomes composed of dipalmitoylphosphatidylcholine, cholesterol and phosphotidylethanolamine are prepared. Modulators of Notch signalling may then be coupled to the phosphatidylethanolamine by the cross-linking agent N-hydroxysuccinimidyl 3-(2-pyridyldithio)propionate. The coupling of the fragment to the liposome can be demonstrated by the release of a pre-trapped marker, e.g., carboxyfluorescence, from the liposomes upon the treatment of secondary antibody against the conjugated fragment and complement.

Where the modulator of Notch signalling comprises an IgFc domain it may, for example, be coupled to a liposome or another carrier of the invention via carbohydrate moieties on the Fc domain.

Methods for derivatizing sugar ring moieties to create hydrazide groups for coupling with fragments (and antibodies) are described, for example by Rodwell, J. D. et al., Proc. Nat'l Acad. Sci. USA 83:2632-36 (1986).

The following papers describe various polymers suitable for use with the present invention, especially for formation/coating of particles.

Dextran: In vitro biocompatibility of biodegradable dextran-based hydrogels tested with human fibroblasts, Biomaterials 22 (2001) 1197-1203 De Groot et al. Methacrylate-derivatized dextran is biocompatible and a promising system for drug delivery.

A novel somatostatin conjugate with a high affinity to al five somatostatin receptor subtypes, Cancer 94 (2002) 1293-1297 Wulbrand et al. Somatostatin coupled to periodate-activated dextran has extended half-life, in clinical trials for hormone-refractory prostate cancer.

Synthesis and inverse emulsion polymerization of aminated acrylamidodextran J. Pharm. Pharmacol. 45 (1993) 1018-1023 Daubresse et al. Derivatized dextran with functional amine groups for drug conjugates.

PEG: Copolymer with styrene; Polystyrene-poly (ethylene glycol) (PS-PEG2000) particles as model systems for site specific drug delivery. 2. The effect of PEG surface density on the in vitro cell interaction and in vivo biodistribution. Pharm. Res. 11 (1994) 1016-1022.

PEG coating of beads; PEGylation of microspheres generates a heterogenous population of particles with different surface characteristics and biological performance. FEBS Letts 532 (2002)338-344 Ghadamosi et al. 1 micron polystyrene beads coated with BSA then PEGylated, a population resistant to phagocytosis and reduced complement activation.

Surface characterization of functionalized polylactide through the coating with heterobifunctional poly(ethylene glycol)/polylactide block copolymers. Biomacromolecules 1 (2000) 39-48 Otsuka et al. Surface reactive aldehyde PEG coated onto polylactide.

Design of biodegradable particles for protein delivery. J. Control Release 78 (2002) 15-24 Vila et al. PEG-coated poly(lactide), chitosan-coated poly(lactic acid-glycolic acid), chitosan.

Albumin nanoparticles; Preparation of surface modified protein nanoparticles by introduction of sulfhydryl groups. Int. J. Pharm. 211 (2000) 67-78 Weber et al. Thiol groups introduced to the surface of human serum albumin nanoparticles.

“Stealth nanospheres”; Prolonging the circulation time and modifying the body distribution of intravenously injected polystyrene nanospheres by prior intravenous administration of poloxamine-908. A ‘hepatic-blockade’ event or manipulation of nanosphere surface in vivo? BBA 1336 (1997) 1-6 Moghimi, S. M. 60 or 250 nanometre polystyrene have extended half-life in vivo if coated with poloxamine 908 or poloxamine-protein conjugates.

Chemical camouflage of nanospheres with a poorly reactive surface: towards development of stealth and target-specific nanocarriers. BBA 1590 (2002) 131-139 Moghimi, S. M. Coating of polystyrene nanospheres with PEGylated BSA or IgG.

Capture of Stealth Nanoparticles by the Body's Defenses Crit. Rev. Ther. Drug Car. Syst. 18 (2001) 527-550 Moghimi S. M. and Hunter, A. C. Review.

Polypeptide Sequences

As used herein, the term “polypeptide” is synonymous with the term “amino acid sequence” and/or the term “protein”. In some instances, the term “polypeptide” is synonymous with the term “peptide”.

“Peptide” usually refers to a short amino acid sequence that is 10 to 40 amino acids long, preferably 10 to 35 amino acids.

The polypeptide sequence may be prepared and isolated from a suitable source, or it may be made synthetically or it may be prepared by use of recombinant DNA techniques.

Polynucleotide Sequences

As used herein, the term “polynucleotide sequence” is synonymous with the term “polynucleotide” and/or the term “nucleotide sequence”.

The polynucleotide sequence may be DNA or RNA of genomic or synthetic or of recombinant origin. They may also be cloned by standard techniques. The polynucleotide sequence may be double-stranded or single-stranded whether representing the sense or antisense strand or combinations thereof.

“Polynucleotide” refers to a polymeric form of nucleotides of at least 10 bases in length and up to 1,000 bases or even more. Longer polynucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

The nucleic acid may be RNA or DNA and is preferably DNA. Where it is RNA, manipulations may be performed via cDNA intermediates. Generally, a nucleic acid sequence encoding the first region will be prepared and suitable restriction sites provided at the 5′ and/or 3′ ends. Conveniently the sequence is manipulated in a standard laboratory vector, such as a plasmid vector based on pBR322 or pUC19 (see below). Reference is made to Molecular Cloning by Sambrook et al. (Cold Spring Harbor, 1989) or similar standard reference books for exact details of the appropriate techniques.

Sources of nucleic acid may be ascertained by reference to published literature or databanks such as GenBank. Nucleic acid encoding the desired first or second sequences may be obtained from academic or commercial sources where such sources are willing to provide the material or by synthesising or cloning the appropriate sequence where only the sequence data are available. Generally this may be done by reference to literature sources which describe the cloning of the gene in question.

Alternatively, where limited sequence data is available or where it is desired to express a nucleic acid homologous or otherwise related to a known nucleic acid, exemplary nucleic acids can be characterised as those nucleotide sequences which hybridise to the nucleic acid sequences known in the art.

The polynucleotide sequence may comprise, for example, a protein-encoding domain, an antisense sequence or a functional motif such as a protein-binding domain and includes variants, derivatives, analogues and fragments thereof. The term also refers to polypeptides encoded by the nucleotide sequence.

Variants, Derivatives, Analogues, Homologues and Fragments

In addition to the specific polypeptide and polynucleotide sequences mentioned herein, the present invention also encompasses the use of variants, derivatives, analogues, homologues, mimetics and fragments thereof.

In the context of the present invention, a variant of any given sequence is a sequence in which the specific sequence of residues (whether amino acid or nucleic acid residues) has been modified in such a manner that the polypeptide or polynucleotide in question retains at least one of its endogenous functions. A variant sequence can be modified by addition, deletion, substitution modification replacement and/or variation of at least one residue present in the naturally-occurring protein.

The term “derivative” as used herein, in relation to proteins or polypeptides of the present invention includes any substitution of, variation of, modification of, replacement of, deletion of and/or addition of one (or more) amino acid residues from or to the sequence providing that the resultant protein or polypeptide retains at least one of its endogenous functions.

The term “analogue” as used herein, in relation to polypeptides or polynucleotides, includes any polypeptide or polynucleotide which retains at least one of the functions of the endogenous polypeptide or polynucleotide but generally has a different evolutionary origin thereto.

The term “mimetic” as used herein, in relation to polypeptides or polynucleotides, refers to a chemical compound that possesses at least one of the endogenous functions of the polypeptide or polynucleotide which it mimics.

Typically, amino acid substitutions may be made, for example from 1, 2 or 3 to 10 or 20 substitutions provided that the modified sequence retains the required transport activity or ability to modulate Notch signalling. Amino acid substitutions may include the use of non-naturally occurring analogues.

Proteins of use in the present invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity, and/or the amphipathic nature of the residues as long as the transport or modulation function is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

For ease of reference, the one and three letter codes for the main naturally occurring amino acids (and their associated codons) are set out below:

| | | | |
|---|---|---|---|
| Symbol | 3-letter | Meaning | Codons |

| | | | |
|---|---|---|---|
| A | Ala | Alanine | GCT, GCC, GCA, GCG |
| B | Asp, Asn | Aspartic, | GAT, GAC, AAT, AAC |
| | | Asparagine | |
| C | Cys | Cysteine | TGT, TGC |
| D | Asp | Aspartic | GAT, GAC |
| E | Glu | Glutamic | GAA, GAG |
| F | Phe | Phenylalanine | TTT, TTC |
| G | Gly | Glycine | GGT, GGC, GGA, GGG |
| H | His | Histidine | CAT, CAC |
| I | Ile | Isoleucine | ATT, ATC, ATA |
| K | Lys | Lysine | AAA, AAG |
| L | Leu | Leucine | TTG, TTA, CTT, CTC, |
| | | | CTA, CTG |
| M | Met | Methionine | ATG |
| N | Asn | Asparagine | AAT, AAC |
| P | Pro | Proline | CCT, CCC, CCA, CCG |
| Q | Gln | Glutamine | CAA, CAG |
| R | Arg | Arginine | CGT, CGC, CGA, CGG, |
| | | | AGA, AGG |
| S | Ser | Serine | TCT, TCC, TCA, TCG, |
| | | | AGT, AGC |
| T | Thr | Threonine | ACT, ACC, ACA, ACG |
| V | Val | Valine | GTT, GTC, GTA, GTG |
| W | Trp | Tryptophan | TGG |
| X | Xxx | Unknown | |
| Y | Tyr | Tyrosine | TAT, TAC |
| Z | Glu, Gln | Glutamic, | GAA, GAG, CAA, CAG |
| | | Glutamine | |
| * | End | Terminator | TAA, TAG, TGA |

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

As used herein, the term “protein” includes single-chain polypeptide molecules as well as multiple-polypeptide complexes where individual constituent polypeptides are linked by covalent or non-covalent means. As used herein, the terms “polypeptide” and “peptide” refer to a polymer in which the monomers are amino acids and are joined together through peptide or disulfide bonds. The terms subunit and domain may also refer to polypeptides and peptides having biological function.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleodtide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Polynucleotide variants will preferably comprise codon optimised sequences. Codon optimisation is known in the art as a method of enhancing RNA stability and therefor gene expression. The redundancy of the genetic code means that several different codons may encode the same amino-acid. For example, Leucine, Arginine and Serine are each encoded by six different codons. Different organisms show preferences in their use of the different codons. Viruses such as HIV, for instance, use a large number of rare codons. By changing a nucleotide sequence such that rare codons are replaced by the corresponding commonly used mammalian codons, increased expression of the sequences in mammalian target cells can be achieved. Codon usage tables are known in the art for mammalian cells, as well as for a variety of other organisms. Preferably, at least part of the sequence is codon optimised. Even more preferably, the sequence is codon optimised in its entirety.

As used herein, the term “homology” can be equated with “identity”. An homologous sequence will be taken to include an amino acid sequence which may be at least 75, 85 or 90% identical, preferably at least 95 or 98% identical. In particular, homology should typically be considered with respect to those regions of the sequence (such as amino acids at positions 51, 56 and 57) known to be essential for an activity. Although homology can also be considered in terms of similarity (i.e. amino acid residues having similar chemical properties/functions), in the context of the present invention it is preferred to express homology in terms of sequence identity.

Homology comparisons can be conducted by eye, or more usually, with the aid of readily available sequence comparison programs. These commercially available computer programs can calculate % homology between two or more sequences.

Percent homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package (see below) the default gap penalty for amino acid sequences is −12 for a gap and −4 for each extension.

Calculation of maximum % homology therefor firstly requires the production of an optimal alignment, taking into consideration gap penalties. A suitable computer program for carrying out such an alignment is the GCG Wisconsin Bestfit package (Devereux). Examples of other software than can perform sequence comparisons include, but are not limited to, the BLAST package, FASTA (Atschul) and the GENEWORKS suite of comparison tools. Both BLAST and FASTA are available for offline and online searching. However it is preferred to use the GCG Bestfit program.

Although the final % homology can be measured in terms of identity, the alignment process itself is typically not based on an all- or -nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). It is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

Nucleotide sequences which are homologous to or variants of sequences of use in the present invention can be obtained in a number of ways, for example by probing DNA libraries made from a range of sources. In addition, other viral/bacterial, or cellular homologues particularly cellular homologues found in mammalian cells (e.g. rat, mouse, bovine and primate cells), may be obtained and such homologues and fragments thereof in general will be capable of selectively hybridising to the sequences shown in the sequence listing herein. Such sequences may be obtained by probing cDNA libraries made from or genomic DNA libraries from other animal species, and probing such libraries with probes comprising all or part of the reference nucleotide sequence under conditions of medium to high stringency. Similar considerations apply to obtaining species homologues and allelic variants of the amino acid and/or nucleotide sequences useful in the present invention.

Variants and strain/species homologues may also be obtained using degenerate PCR which will use primers designed to target sequences within the variants and homologues encoding conserved amino acid sequences within the sequences of use in the present invention. Conserved sequences can be predicted, for example, by aligning the amino acid sequences from several variants/homologues. Sequence alignments can be performed using computer software known in the art. For example the GCG Wisconsin PileUp program is widely used. The primers used in degenerate PCR will contain one or more degenerate positions and will be used at stringency conditions lower than those used for cloning sequences with single sequence primers against known sequences.

Alternatively, such nucleotide sequences may be obtained by site directed mutagenesis of characterised sequences. This may be useful where for example silent codon changes are required to sequences to optimise codon preferences for a particular host cell in which the nucleotide sequences are being expressed. Other sequence changes may be desired in order to introduce restriction enzyme recognition sites, or to alter the activity of the polynucleotide or encoded polypeptide.

In general, primers will be produced by synthetic means, involving a step wise manufacture of the desired nucleic acid sequence one nucleotide at a time. Techniques for accomplishing this using automated techniques are readily available in the art.

Longer nucleotide sequences will generally be produced using recombinant means, for example using a PCR (polymerase chain reaction) cloning techniques. This will involve making a pair of primers (e.g. of about 15 to 30 nucleotides) flanking a region of the targeting sequence which it is desired to clone, bringing the primers into contact with mRNA or cDNA obtained from an animal or human cell, performing a polymerase chain reaction (PCR) under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

For recombinant production, host cells can be genetically engineered to incorporate expression systems or polynucleotides of the invention. Introduction of a polynucleotide into the host cell can be effected by methods described in many standard laboratory manuals, such as Davis et al and Sambrook et al, such as calcium phosphate transfection, DEAE-dextran mediated transfection, transfection, microinjection, cationic lipid-mediated transfection, electroporation, transduction, scrape loading, ballistic introduction and infection. It will be appreciated that such methods can be employed in vitro or in vivo as drug delivery systems.

Representative examples of appropriate hosts include bacterial cells, such as streptococci, staphylococci, E. coli, streptomyces and Bacillus subtilis cells; fungal cells, such as yeast cells and Aspergillus cells; insect cells such as Drosophila S2 and Spodoptera Sf9 cells; animal cells such as CHO, COS, NSO, HeLa, C127, 3T3, BHK, 293 and Bowes melanoma cells; and plant cells.

A great variety of expression systems can be used to produce a polypeptide useful in the present invention. Such vectors include, among others, chromosomal, episomal and virus-derived vectors, e.g., vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. The expression system constructs may contain control regions that regulate as well as engender expression. Generally, any system or vector suitable to maintain, propagate or express polynucleotides and/or to express a polypeptide in a host may be used for expression in this regard. The appropriate DNA sequence may be inserted into the expression system by any of a variety of well-known and routine techniques, such as, for example, those set forth in Sambrook et al.

For secretion of the translated protein into the lumen of the endoplasmic reticulum, into the periplasmic space or into the extracellular environment, appropriate secretion signals may be incorporated into the expressed polypeptide. These signals may be endogenous to the polypeptide or they may be heterologous signals.

Active agents for use in the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Most preferably, high performance liquid chromatography is employed for purification. Well known techniques for refolding protein may be employed to regenerate active conformation when the polypeptide is denatured during isolation and/or purification.

In one embodiment of the invention, any one or more of the above candidate modulators is brought into contact with a cell of the immune system. Cells of the immune system of use in the present invention are described below.

Cells of the Immune System

Cells of use in the present invention are cells of the immune system capable of transducing the Notch signalling pathway.

Most preferably the cells of use in the present invention are T-cells. These include, but are not limited to, CD4⁺ and CD8⁺ mature T cells, immature T cells of peripheral or thymic origin and NK-T cells.

Alternatively, the cells will be antigen-presenting cells (APCs). APCs include dendritic cells (DCs) such as interdigitating DCs or follicular DCs, Langerhans cells, PBMCs, macrophages, B-lymphocytes, T-lymphocytes, or other cell types such as epithelial cells, fibroblasts or endothelial cells, constitutively expressing or activated to express a MHC Class II molecules on their surfaces. Precursors of APCs include CD34⁺ cells, monocytes, fibroblasts and endothelial cells. The APCs or precursors may be modified by the culture conditions or may be genetically modified, for instance by transfection of one or more genes. The T cells or APCs may be isolated from a patient, or from a donor individual or another individual. The cells are preferably mammalian cells such as human or mouse cells. Preferably the cells are of human origin. The APC or precursor APC may be provided by a cell proliferating in culture such as an established cell line or a primary cell culture. Examples include hybridoma cell lines, L-cells and human fibroblasts such as MRC-5. Preferred cell lines for use in the present invention include Jurkat, H9, CEM and EL4 T-cells; long-term T-cell clones such as human HA1.7 or mouse D10 cells; T-cell hybridomas such as DO11.10 cells; macrophage-like cells such as U937 or THP1 cells; B-cell lines such as EBV-transformed cells such as Raji, A20 and M1 cells.

Dendritic cells (DCs) can be isolated/prepared by a number of means, for example they can either be purified directly from peripheral blood, or generated from CD34⁺ precursor cells for example after mobilisation into peripheral blood by treatment with GM-CSF, or directly from bone marrow. From peripheral blood, adherent precursors can be treated with a GM-CSF/IL-4 mixture (Inaba et al), or from bone marrow, non-adherent CD34⁺ cells can be treated with GM-CSF and TNF-α (Caux et al). DCs can also be routinely prepared from the peripheral blood of human volunteers, similarly to the method of Sallusto and Lanzavecchia J Exp Med (1994) 179(4) 1109-18 using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin et al). Culture conditions may include other cytokines such as GM-CSF or IL-4 for the maintenance and, or activity of the dendritic cells or other antigen presenting cells.

T cells and B cells for use in the invention are preferably obtained from cell lines such as lymphoma or leukemia cell lines, T cell hybridomas or B cell hybridomas but may also be isolated from an individual suffering from a disease of the immune system or a recipient for a transplant operation or from a related or unrelated donor individual. T cells and B cells may be obtained from blood or another source (such as lymph nodes, spleen, or bone marrow) and may be enriched or purified by standard procedures. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used.

Candidate modulators of use in the present invention are brought into contact with a cell of the immune system as described above. In a further step, modulation of Notch signalling by a candidate modulator is detected. Assays for detecting modulation of Notch signalling will be described below. Many of these assays will involve monitoring the expression of a “target gene”.

Notch Signalling Pathway

Endogenous target genes of the Notch signalling pathway include Deltex, genes of the Hes family (Hes-1 in particular), Enhancer of Split [E(spl)] complex genes, Il-10, CD-23, Dlx-1, CTLA4, CD-4, Dll-1, Numb, Mastermind and Dsh. Although all genes the expression of which is modulated by Notch activation may be used for the purpose of the present invention, preferred endogenous target genes are described below.

Deltex, an intracellular docking protein, replaces Su(H) as it leaves its site of interaction with the intracellular tail of Notch, as shown in FIG. 1. Deltex is a cytoplasmic protein containing a zinc-finger (Artavanis-Tsakonas; Osborne). It interacts with the ankyrin repeats of the Notch intracellular domain. Studies indicate that Deltex promotes Notch pathway activation by interacting with Grb2 and modulating the Ras-JNK signalling pathway (Matsuno). Deltex also acts as a docking protein which prevents Su(H) from binding to the intracellular tail of Notch (Matsuno). Thus, Su(H) is released into the nucleus where it acts as a transcriptional modulator. Recent evidence also suggests that, in a vertebrate B-cell system, Deltex, rather than the Su(H) homologue CBF1, is responsible for inhibiting E47 function (Ordentlich). Expression of Deltex is upregulated as a result of Notch activation in a positive feedback loop. The sequence of Homo sapiens Deltex (DTX1) mRNA may be found in GenBank Accession No. AF053700.

Hes-1 (Hairy-enhancer of Split-1) (Takebayashi) is a transcriptional factor with a basic helix-loop-helix structure. It binds to an important functional site in the CD4 silencer leading to repression of CD4 gene expression. Thus, Hes-1 is strongly involved in the determination of T-cell fate. Other genes from the Hes family include Hes-5 (mammalian Enhancer of Split homologue), the expression of which is also upregulated by Notch activation, and Hes-3. Expression of Hes-1 is upregulated as a result of Notch activation. The sequence of human Hes-1 can be found in GenBank Accession Nos. AK000415 and AF264785.

The E(spl) gene complex [E(spl)-C] (Leimeister) comprises seven genes of which only E(spl) and Groucho show visible phenotypes when mutant. E(spl) was named after its ability to enhance Split mutations, Split being another name for Notch. Indeed, E(spl)-C genes repress Delta through regulation of achaete-scute complex gene expression. Expression of E(spl) is upregulated as a result of Notch activation.

IL-10 (interleukin-10) is a factor produced by Th2 helper T-cells. It is a co-regulator of mast cell growth and shows extensive homology with the Epstein-Barr bcrfi gene. Although it is not known to be a direct downstream target of the Notch signalling pathway, its expression has been found to be strongly upregulated coincident with Notch activation. The mRNA sequence of IL-10 may be found in GenBank ref. No. GI1041812.

CD-23 is the human leukocyte differentiation antigen CD23 (FCE2) which is a key molecule for B-cell activation and growth. It is the low-affinity receptor for IgE. Furthermore, the truncated molecule can be secreted, then functioning as a potent mitogenic growth factor. Although it is not thought to be a direct downstream target of the Notch signalling pathway, its expression has been found to be strongly upregulated coincident with Notch activation. The sequence for CD-23 may be found in GenBank ref. No. GI1783344.

Dlx-1 (distalless-1) expression is downregulated as a result of Notch activation. Sequences for Dlx genes may be found in GenBank Accession Nos. U51000-3.

CTLA4 (cytotoxic T-lymphocyte activated protein 4) is an accessory molecule found on the surface of T-cells which is thought to play a role in the regulation of airway inflammatory cell recruitment and T-helper cell differentiation after allergen inhalation. The promoter region of the gene encoding CTLA4 has CBF1 response elements and its expression is upregulated as a result of Notch activation. The sequence of CTLA4 can be found in GenBank Accession No. L15006.

CD-4 expression is downregulated as a result of Notch activation. A sequence for the CD-4 antigen may be found in GenBank Accession No. XM006966.

Assays

Assays for monitoring expression of the one or more target genes and other methods of detecting modulation of Notch signalling are described below.

The assay of the present invention is set up to detect either inhibition or enhancement of Notch signalling in cells of the immune system by candidate modulators. The method comprises mixing cells of the immune system, where necessary transformed or transfected, etc. with a synthetic reporter gene, in an appropriate buffer, with a sufficient amount of candidate modulator and monitoring Notch signalling. The modulators may be small molecules, proteins, antibodies or other ligands as described above. Amounts or activity of the target gene (also described above) will be measured for each compound tested using standard assay techniques and appropriate controls. Preferably the detected signal is compared with a reference signal and any modulation with respect to the reference signal measured.

The assay may also be run in the presence of a known antagonist of the Notch signalling pathway in order to identify compounds capable of rescuing the Notch signal.

Any one or more of appropriate targets—such as an amino acid sequence and/or nucleotide sequence—may be used for identifying a compound capable of modulating the Notch signalling pathway in cells of the immune system in any of a variety of drug screening techniques. The target employed in such a test may be free in solution, affixed to a solid support, borne on a cell surface, or located intracellularly. The assay of the present invention is a cell based assay.

The assay of the present invention may be a screen, whereby a number of agents are tested. In one aspect, the assay method of the present invention is a high through put screen.

Techniques for drug screening may be based on the method described in Geysen, European Patent No. 0138855, published on Sep. 13, 1984. In summary, large numbers of different small peptide candidate modulators are synthesized on a solid substrate, such as plastic pins or some other surface. The peptide test compounds are reacted with a suitable target or fragment thereof and washed. Bound entities are then detected—such as by appropriately adapting methods well known in the art. A purified target can also be coated directly onto plates for use in drug screening techniques. Plates of use for high throughput screening (HTS) will be multi-well plates, preferably having 96, 384 or over 384 wells/plate. Cells can also be spread as “lawns”. Alternatively, non-neutralising antibodies can be used to capture the peptide and immobilise it on a solid support. High throughput screening, as described above for synthetic compounds, can also be used for identifying organic candidate modulators.

This invention also contemplates the use of competitive drug screening assays in which neutralising antibodies capable of binding a target specifically compete with a test compound for binding to a target.

It is expected that the assay methods of the present invention will be suitable for both small and large-scale screening of test compounds as well as in quantitative assays.

Various nucleic acid assays are also known. Any conventional technique which is known or which is subsequently disclosed may be employed. Examples of suitable nucleic acid assay are mentioned below and include amplification, PCR, RT-PCR, RNase protection, blotting, spectrometry, reporter gene assays, gene chip arrays and other hybridization methods.

Target gene presence, amplification and/or expression may be measured in a sample directly, for example, by conventional Southern blotting, Northern blotting to quantitate the transcription of target mRNA, dot blotting (DNA or RNA analysis), or in situ hybridisation, using an appropriately labelled probe. Those skilled in the art will readily envisage how these methods may be modified, if desired.

Generation of nucleic acids for analysis from samples generally requires nucleic acid amplification. Many amplification methods rely on an enzymatic chain reaction (such as a polymerase chain reaction, a ligase chain reaction, or a self-sustained sequence replication) or from the replication of all or part of the vector into which it has been cloned. Preferably, the amplification according to the invention is an exponential amplification, as exhibited by for example the polymerase chain reaction.

Many target and signal amplification methods have been described in the literature, for example, general reviews of these methods in Landegren, U., et al., Science 242:229-237 (1988) and Lewis, R., Genetic Engineering News 10:1, 54-55 (1990). These amplification methods may be used in the methods of our invention, and include polymerase chain reaction (PCR), PCR in situ, ligase amplification reaction (LAR), ligase hybridisation, Qbeta bacteriophage replicase, transcription-based amplification system (TAS), genomic amplification with transcript sequencing (GAWTS), nucleic acid sequence-based amplification (NASBA) and in situ hybridisation. Primers suitable for use in various amplification techniques can be prepared according to methods known in the art.

PCR is a nucleic acid amplification method described inter alia in U.S. Pat. Nos. 4,683,195 and 4,683,202. PCR consists of repeated cycles of DNA polymerase generated primer extension reactions. PCR was originally developed as a means of amplifying DNA from an impure sample. The technique is based on a temperature cycle which repeatedly heats and cools the reaction solution allowing primers to anneal to target sequences and extension of those primers for the formation of duplicate daughter strands. RT-PCR uses an RNA template for generation of a first strand cDNA with a reverse transcriptase. The cDNA is then amplified according to standard PCR protocol. Repeated cycles of synthesis and denaturation result in an exponential increase in the number of copies of the target DNA produced. However, as reaction components become limiting, the rate of amplification decreases until a plateau is reached and there is little or no net increase in PCR product. The higher the starting copy number of the nucleic acid target, the sooner this “end-point” is reached. PCR can be used to amplify any known nucleic acid in a diagnostic context (Mok et al., (1994), Gynaecologic Oncology, 52: 247-252).

Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al. (1990) Proc. Natl. Acad. Sci. USA 87:1874). Enzymatic degradation of the RNA of the RNA/DNA heteroduplex is used instead of heat denaturation. RNase H and all other enzymes are added to the reaction and all steps occur at the same temperature and without further reagent additions. Following this process, amplifications of 10⁶ to 10⁹ have been achieved in one hour at 42° C.

Ligation amplification reaction or ligation amplification system uses DNA ligase and four oligonucleotides, two per target strand. This technique is described by Wu, D. Y. and Wallace, R. B. (1989) Genomics 4:560. The oligonucleotides hybridise to adjacent sequences on the target DNA and are joined by the ligase. The reaction is heat denatured and the cycle repeated.

Alternative amplification technology can be exploited in the present invention. For example, rolling circle amplification (Lizardi et al., (1998) Nat Genet 19:225) is an amplification technology available commercially (RCA™) which is driven by DNA polymerase and can replicate circular oligonucleotide probes with either linear or geometric kinetics under isothermal conditions.

In the presence of two suitably designed primers, a geometric amplification occurs via DNA strand displacement and hyperbranching to generate 10¹² or more copies of each circle in 1 hour.

If a single primer is used, RCAT generates in a few minutes a linear chain of thousands of tandemly linked DNA copies of a target covalently linked to that target.

A further technique, strand displacement amplification (SDA; Walker et al., (1992) PNAS (USA) 80:392) begins with a specifically defined sequence unique to a specific target. But unlike other techniques which rely on thermal cycling, SDA is an isothermal process that utilises a series of primers, DNA polymerase and a restriction enzyme to exponentially amplify the unique nucleic acid sequence.

SDA comprises both a target generation phase and an exponential amplification phase.

In target generation, double-stranded DNA is heat denatured creating two single-stranded copies. A series of specially manufactured primers combine with DNA polymerase (amplification primers for copying the base sequence and bumper primers for displacing the newly created strands) to form altered targets capable of exponential amplification.

The exponential amplification process begins with altered targets (single-stranded partial DNA strands with restricted enzyme recognition sites) from the target generation phase.

An amplification primer is bound to each strand at its complementary DNA sequence. DNA polymerase then uses the primer to identify a location to extend the primer from its 3′ end, using the altered target as a template for adding individual nucleotides. The extended primer thus forms a double-stranded DNA segment containing a complete restriction enzyme recognition site at each end.

A restriction enzyme is then bound to the double stranded DNA segment at its recognition site. The restriction enzyme dissociates from the recognition site after having cleaved only one strand of the double-sided segment, forming a nick. DNA polymerase recognises the nick and extends the strand from the site, displacing the previously created strand. The recognition site is thus repeatedly nicked and restored by the restriction enzyme and DNA polymerase with continuous displacement of DNA strands containing the target segment. Each displaced strand is then available to anneal with amplification primers as above. The process continues with repeated nicking, extension and displacement of new DNA strands, resulting in exponential amplification of the original DNA target.

In an alternative embodiment, the present invention provides for the detection of gene expression at the RNA level. Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Real-time PCR uses probes labeled with a fluorescent tag or fluorescent dyes and differs from end-point PCR for quantitative assays in that it is used to detect PCR products as they accumulate rather than for the measurement of product accumulation after a fixed number of cycles. The reactions are characterized by the point in time during cycling when amplification of a target sequence is first detected through a significant increase in fluorescence.

The ribonuclease protection (RNase protection) assay is an extremely sensitive technique for the quantitation of specific RNAs in solution. The ribonuclease protection assay can be performed on total cellular RNA or poly(A)-selected mRNA as a target. The sensitivity of the ribonuclease protection assay derives from the use of a complementary in vitro transcript probe which is radiolabeled to high specific activity. The probe and target RNA are hybridized in solution, after which the mixture is diluted and treated with ribonuclease (RNase) to degrade all remaining single-stranded RNA. The hybridized portion of the probe will be protected from digestion and can be visualized via electrophoresis of the mixture on a denaturing polyacrylamide gel followed by autoradiography. Since the protected fragments are analyzed by high resolution polyacrylamide gel electrophoresis, the ribonuclease protection assay can be employed to accurately map mRNA features. If the probe is hybridized at a molar excess with respect to the target RNA, then the resulting signal will be directly proportional to the amount of complementary RNA in the sample.

PCR technology as described e.g. in section 14 of Sambrook et al., 1989, requires the use of oligonucleotide probes that will hybridise to target nucleic acid sequences. Strategies for selection of oligonucleotides are described below.

As used herein, a probe is e.g. a single-stranded DNA or RNA that has a sequence of nucleotides that includes between 10 and 50, preferably between 15 and 30 and most preferably at least about 20 contiguous bases that are the same as (or the complement of) an equivalent or greater number of contiguous bases. The nucleic acid sequences selected as probes should be of sufficient length and sufficiently unambiguous so that false positive results are minimised. The nucleotide sequences are usually based on conserved or highly homologous nucleotide sequences or regions of polypeptides. The nucleic acids used as probes may be degenerate at one or more positions.

Preferred regions from which to construct probes include 5′ and/or 3′ coding sequences, sequences predicted to encode ligand binding sites, and the like. For example, either the full-length cDNA clone disclosed herein or fragments thereof can be used as probes. Preferably, nucleic acid probes of the invention are labelled with suitable label means for ready detection upon hybridisation. For example, a suitable label means is a radiolabel. The preferred method of labelling a DNA fragment is by incorporating ³²P dATP with the Klenow fragment of DNA polymerase in a random priming reaction, as is well known in the art. Oligonucleotides are usually end-labelled with ³²P-labelled ATP and polynucleotide kinase. However, other methods (e.g. non-radioactive) may also be used to label the fragment or oligonucleotide, including e.g. enzyme labelling, fluorescent labelling with suitable fluorophores and biotinylation.

Preferred are such sequences, probes which hybridise under high-stringency conditions.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of higher stringency, followed by washes of varying stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6×SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1×SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the hybridising pair also play a role.

Gene expression may also be detected using a reporter system. Such a reporter system may comprise a readily identifiable marker under the control of an expression system, e.g. of the gene being monitored. Fluorescent markers, which can be detected and sorted by FACS, are preferred. Especially preferred are GFP and luciferase. Another type of preferred reporter is cell surface markers, i.e. proteins expressed on the cell surface and therefor easily identifiable. Thus, cell-based screening assays can be designed by constructing cell lines in which the expression of a reporter protein, i.e. an easily assayable protein, such as β-galactosidase, chloramphenicol acetyltransferase (CAT) or luciferase, is dependent on the activation of a Notch. For example, a reporter gene encoding one of the above polypeptides may be placed under the control of an response element which is specifically activated by Notch signalling. Alternative assay formats include assays which directly assess responses in a biological system. If a cell-based assay system is employed, the test compound(s) indentified may then be subjected to in vivo testing to determine their effect on Notch signalling pathway.

In general, reporter constructs useful for detecting Notch signalling by expression of a reporter gene may be constructed according to the general teaching of Sambrook et al (1989). Typically, constructs according to the invention comprise a promoter of the gene of interest (i.e. of an endogenous target gene), and a coding sequence encoding the desired reporter constructs, for example of GFP or luciferase. Vectors encoding GFP and luciferase are known in the art and available commercially.

Sorting of cells, based upon detection of expression of target genes, may be performed by any technique known in the art, as exemplified above. For example, cells may be sorted by flow cytometry or FACS. For a general reference, see Flow Cytometry and Cell Sorting: A Laboratory Manual (1992) A. Radbruch (Ed.), Springer Laboratory, New York.

Flow cytometry is a powerful method for studying and purifying cells. It has found wide application, particularly in immunology and cell biology: however, the capabilities of the FACS can be applied in many other fields of biology. The acronym F.A.C.S. stands for Fluorescence Activated Cell Sorting, and is used interchangeably with “flow cytometry”. The principle of FACS is that individual cells, held in a thin stream of fluid, are passed through one or more laser beams, causing light to be scattered and fluorescent dyes to emit light at various frequencies. Photomultiplier tubes (PMT) convert light to electrical signals, which are interpreted by software to generate data about the cells. Sub-populations of cells with defined characteristics can be identified and automatically sorted from the suspension at very high purity (˜100%).

FACS can be used to measure target gene expression in cells transfected with recombinant DNA encoding polypeptides. This can be achieved directly, by labelling of the protein product, or indirectly by using a reporter gene in the construct. Examples of reporter genes are β-galactosidase and Green Fluorescent Protein (GFP). β-galactosidase activity can be detected by FACS using fluorogenic substrates such as fluorescein digalactoside (FDG). FDG is introduced into cells by hypotonic shock, and is cleaved by the enzyme to generate a fluorescent product, which is trapped within the cell. One enzyme can therefor generate a large amount of fluorescent product. Cells expressing GFP constructs will fluoresce without the addition of a substrate. Mutants of GFP are available which have different excitation frequencies, but which emit fluorescence in the same channel. In a two-laser FACS machine, it is possible to distinguish cells which are excited by the different lasers and therefor assay two transfections at the same time.

Alternative means of cell sorting may also be employed. For example, the invention comprises the use of nucleic acid probes complementary to mRNA. Such probes can be used to identify cells expressing polypeptides individually, such that they may subsequently be sorted either manually, or using FACS sorting. Nucleic acid probes complementary to mRNA may be prepared according to the teaching set forth above, using the general procedures as described by Sambrook et al (1989).

In a preferred embodiment, the invention comprises the use of an antisense nucleic acid molecule, complementary to a target mRNA, conjugated to a fluorophore which may be used in FACS cell sorting.

Methods have also been described for obtaining information about gene expression and identity using so-called gene chip arrays or high density DNA arrays (Chee). These high density arrays are particularly useful for diagnostic and prognostic purposes. Use may also be made of In vivo Expression Technology (IVET) (Camilli). IVET identifies target genes up-regulated during say treatment or disease when compared to laboratory culture.

The present invention also provides a method of detection of polypeptides. The advantage of using a protein assay is that Notch activation can be directly measured. Assay techniques that can be used to determine levels of a polypeptide are well known to those skilled in the art. Such assay methods include radioimmunoassays, competitive-binding assays, protein gel assay, Western Blot analysis, antibody sandwich assays, antibody detection, FACS and ELISA assays. For example, polypeptides can be detected by differential mobility on protein gels, or by other size analysis techniques, such as mass spectrometry. The detection means may be sequence-specific. For example, polypeptide or RNA molecules can be developed which specifically recognise polypeptides in vivo or in vitro.

For example, RNA aptamers can be produced by SELEX. SELEX is a method for the in vitro evolution of nucleic acid molecules with highly specific binding to target molecules. It is described, for example, in U.S. Pat. Nos. 5,654,151, 5,503,978, 5,567,588 and 5,270,163, as well as PCT publication WO 96/38579.

The invention, in certain embodiments, includes antibodies specifically recognising and binding to polypeptides.

Antibodies may be recovered from the serum of immunised animals. Monoclonal antibodies may be prepared from cells from immunised animals in the conventional manner.

The antibodies of the invention are useful for identifying cells expressing the genes being monitored.

Antibodies according to the invention may be whole antibodies of natural classes, such as IgE and IgM antibodies, but are preferably IgG antibodies. Moreover, the invention includes antibody fragments, such as Fab, F(ab′)2, Fv and ScFv. Small fragments, such Fv and ScFv, possess advantageous properties for diagnostic and therapeutic applications on account of their small size and consequent superior tissue distribution.

The antibodies may comprise a label. Especially preferred are labels which allow the imaging of the antibody in neural cells in vivo. Such labels may be radioactive labels or radioopaque labels, such as metal particles, which are readily visualisable within tissues. Moreover, they may be fluorescent labels or other labels which are visualisable in tissues and which may be used for cell sorting.

In more detail, antibodies as used herein can be altered antibodies comprising an effector protein such as a label. Especially preferred are labels which allow the imaging of the distribution of the antibody in vivo. Such labels can be radioactive labels or radioopaque labels, such as metal particles, which are readily visualisable within the body of a patient. Moreover, they can be fluorescent labels or other labels which are visualisable on tissue.

Antibodies as described herein can be produced in cell culture. Recombinant DNA technology can be used to produce the antibodies according to established procedure, in bacterial or preferably mammalian cell culture. The selected cell culture system optionally secretes the antibody product, although antibody products can be isolated from non-secreting cells.

Multiplication of hybridoma cells or mammalian host cells in vitro is carried out in suitable culture media, which are the customary standard culture media, for example Dulbecco's Modified Eagle Medium (DMEM) or RPMI 1640 medium, optionally replenished by a mammalian serum, e.g. foetal calf serum, or trace elements and growth sustaining supplements, e.g. feeder cells such as normal mouse peritoneal exudate cells, spleen cells, bone marrow macrophages, 2-aminoethanol, insulin, transferrin, low density lipoprotein, oleic acid, or the like. Multiplication of host cells which are bacterial cells or yeast cells is likewise carried out in suitable culture media known in the art, for example for bacteria in medium LB, NZCYM, NZYM, NZM, Terrific Broth, SOB, SOC, 2×YT, or M9 Minimal Medium, and for yeast in medium YPD, YEPD, Minimal Medium, or Complete Minimal Dropout Medium.

In vitro production provides relatively pure antibody preparations and allows scale-up to give large amounts of the desired antibodies. Techniques for bacterial cell, yeast or mammalian cell cultivation are known in the art and include homogeneous suspension culture, e.g. in an airlift reactor or in a continuous stirrer reactor, or immobilised or entrapped cell culture, e.g. in hollow fibres, microcapsules, on agarose microbeads or ceramic cartridges.

Large quantities of the desired antibodies can also be obtained by multiplying mammalian cells in vivo. For this purpose, hybridoma cells producing the desired antibodies are injected into histocompatible mammals to cause growth of antibody-producing tumours. Optionally, the animals are primed with a hydrocarbon, especially mineral oils such as pristane (tetramethyl-pentadecane), prior to the injection. After one to three weeks, the antibodies are isolated from the body fluids of those mammals. For example, hybridoma cells obtained by fusion of suitable myeloma cells with antibody-producing spleen cells from Balb/c mice, or transfected cells derived from hybridoma cell line Sp2/0 that produce the desired antibodies are injected intraperitoneally into Balb/c mice optionally pre-treated with pristane, and, after one to two weeks, ascitic fluid is taken from the animals.

The foregoing, and other, techniques are discussed in, for example, Kohler and Milstein, (1975) Nature 256:495-497; U.S. Pat. No. 4,376,110; Harlow and Lane, Antibodies: a Laboratory Manual, (1988) Cold Spring Harbor, incorporated herein by reference. Techniques for the preparation of recombinant antibody molecules is described in the above references and also in, for example, EP 0623679; EP 0368684 and EP 0436597, which are incorporated herein by reference.

The cell culture supernatants are screened for the desired antibodies, preferentially by an enzyme immunoassay, e.g. a sandwich assay or a dot-assay, or a radioimmunoassay.

For isolation of the antibodies, the immunoglobulins in the culture supernatants or in the ascitic fluid can be concentrated, e.g. by precipitation with ammonium sulphate, dialysis against hygroscopic material such as polyethylene glycol, filtration through selective membranes, or the like. If necessary and/or desired, the antibodies are purified by the customary chromatography methods, for example gel filtration, ion-exchange chromatography, chromatography over DEAE-cellulose and/or (immuno-) affinity chromatography, e.g. affinity chromatography with the target antigen, or with Protein-A. The antibody is preferably provided together with means for detecting the antibody, which can be enzymatic, fluorescent, radioisotopic or other means. The antibody and the detection means can be provided for simultaneous, simultaneous separate or sequential use, in a kit.

The antibodies of the invention are assayed for immunospecific binding by any method known in the art. The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al, eds, 1994, Current Protocols in Molecular Biology, Vol. 1, John Wiley & Sons, Inc., New York, which is incorporated by reference herein in its entirety). Exemplary immunoassays are described briefly below.

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), exposing the membrane to a primary antibody (the antibody of interest) diluted in blocking buffer, washing the membrane in washing buffer, exposing the membrane to a secondary antibody (which recognises the primary antibody, e.g., an antihuman antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen.

ELISAs generally comprise preparing antigen, coating the well of a 96 well microtitre plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognises the antibody of interest) conjugated to a detectable compound can be added to the well. Further, instead of coating the well with the antigen, the antibody can be coated to the well. In this case, a second antibody conjugated to a detectable compound can be added following the addition of the antigen of interest to the coated well.

It is convenient when running assays to immobilise one of more of the reactants, particularly when the reactant is soluble. In the present case it may be convenient to immobilise any one of more of the candidate modulator, Notch ligand, immune cell activator or immune cell costimulus. Immobilisation approaches include covalent immobilisation, such as using amine coupling, surface thiol coupling, ligand thiol coupling and aldehyde coupling, and high affinity capture which relies on high affinity binding of a ligand to an immobilised capturing molecule. Example of capturing molecules include: streptavidin, anti-mouse Ig antibodies, ligand-specific antibodies, protian A, protein G and Tag-specific capture. In one embodiment, immobilisation is achieved through binding to a support, particularly a particulate support which is preferably in the form of a bead.

For assays involving monitoring or detection of tolerised T-cells for use in clinical applications, the assay will generally involve removal of a sample from a patient prior to the step of detecting a signal resulting from cleavage of the intracellular domain.

The invention additionally provides a method of screening for a candidate modulator of Notch signalling, the method comprising mixing in a buffer an appropriate amount of Notch, wherein Notch is suitably labelled with detection means for monitoring cleavage of Notch; and a sample of a candidate ligand; and monitoring any cleavage of Notch.

As used herein, the term “sample” refers to a collection of inorganic, organic or biochemical molecules which is either found in nature (e.g., in a biological- or other specimen) or in an artificially-constructed grouping, such as agents which may be found and/or mixed in a laboratory. The biological sample may refer to a whole organism, but more usually to a subset of its tissues, cells or component parts (e.g. body fluids, including but not limited to blood, mucus, saliva and urine).

The present invention provides a method of detecting novel modulators of Notch signalling. The modulators identified may be used as therapeutic agents—i.e. in therapy applications.

Therapy

The term “therapy” includes curative effects, alleviation effects, and prophylactic effects. The therapy may be on humans or animals.

Modulators identified by the assay method of the present invention may be used to treat disorders and/or conditions of the immune system. In particular, the compounds can be used in the treatment of T cell mediated diseases or disorders. A detailed description of the conditions affected by the Notch signalling pathway may be found in our WO98/20142, WO00/36089 and WO/00135990.

Diseased or infectious states that may be described as being mediated by T cells include, but are not limited to, any one or more of asthma, allergy, tumour induced aberrations to the T cell system and infectious diseases such as those caused by Plasmodium species, Microfilariae, Helminths, Mycobacteria, HIV, Cytomegalovirus, Pseudomonas, Toxoplasma, Echinococcus, Haemophilus influenza type B, measles, Hepatitis C or Toxicara. Thus particular conditions that may be treated or prevented which are mediated by T cells include multiple sclerosis, rheumatoid arthritis and diabetes. The present invention may also be used in organ transplantation or bone marrow transplantation. The present invention is also useful in treating immune disorders such as autoimmune disorders or graft rejection such as allograft rejection.

Examples of autoimmune disorders range from organ specific diseases (such as thyroiditis, insulitis, multiple sclerosis, iridocyclitis, uveitis, orchitis, hepatitis, Addison's disease, myasthenia gravis) to systemic illnesses such as rheumatoid arthritis or lupus erythematosus. Other disorders include immune hyperreactivity, such as allergic reactions.

In more detail, organ-specific autoimmune diseases include multiple sclerosis, insulin dependent diabetes mellitus, several forms of anemia (aplastic, hemolytic), autoimmune hepatitis, thyroiditis, insulitis, iridocyclitis, skleritis, uveitis, orchitis, myasthenia gravis, idiopathic thrombocytopenic purpura, inflammatory bowel diseases (Crohn's disease, ulcerative colitis).

Systemic autoimmune diseases include: rheumatoid arthritis, juvenile arthritis, scleroderma and systemic sclerosis, sjogren's syndrom, undifferentiated connective tissue syndrome, antiphospholipid syndrome, different forms of vasculitis (polyarteritis nodosa, allergic granulomatosis and angiitis, Wegner's granulomatosis, Kawasaki disease, hypersensitivity vasculitis, Henoch-Schoenlein purpura, Behcet's Syndrome, Takayasu arteritis, Giant cell arteritis, Thrombangiitis obliterans), lupus erythematosus, polymyalgia rheumatica, essentiell (mixed) cryoglobulinemia, Psoriasis vulgaris and psoriatic arthritis, diffus fasciitis with or without eosinophilia, polymyositis and other idiopathic inflammatory myopathies, relapsing panniculitis, relapsing polychondritis, lymphomatoid granulomatosis, erythema nodosum, ankylosing spondylitis, Reiter's syndrome, different forms of inflammatory dermatitis.

A more extensive list of disorders includes: unwanted immune reactions and inflammation including arthritis, including rheumatoid arthritis, inflammation associated with hypersensitivity, allergic reactions, asthma, systemic lupus erythematosus, collagen diseases and other autoimmune diseases, inflammation associated with atherosclerosis, arteriosclerosis, atherosclerotic heart disease, reperfusion injury, cardiac arrest, myocardial infarction, vascular inflammatory disorders, respiratory distress syndrome or other cardiopulmonary diseases, inflammation associated with peptic ulcer, ulcerative colitis and other diseases of the gastrointestinal tract, hepatic fibrosis, liver cirrhosis or other hepatic diseases, thyroiditis or other glandular diseases, glomerulonephritis or other renal and urologic diseases, otitis or other oto-rhino-laryngological diseases, dermatitis or other dermal diseases, periodontal diseases or other dental diseases, orchitis or epididimo-orchitis, infertility, orchidal trauma or other immune-related testicular diseases, placental dysfunction, placental insufficiency, habitual abortion, eclampsia, pre-eclampsia and other immune and/or inflammatory-related gynaecological diseases, posterior uveitis, intermediate uveitis, anterior uveitis, conjunctivitis, chorioretinitis, uveoretinitis, optic neuritis, intraocular inflammation, e.g. retinitis or cystoid macular oedema, sympathetic ophthalmia, scleritis, retinitis pigmentosa, immune and inflammatory components of degenerative fondus disease, inflammatory components of ocular trauma, ocular inflammation caused by infection, proliferative vitreo-retinopathies, acute ischaemic optic neuropathy, excessive scarring, e.g. following glaucoma filtration operation, immune and/or inflammation reaction against ocular implants and other immune and inflammatory-related ophthalmic diseases, inflammation associated with autoimmune diseases or conditions or disorders where, both in the central nervous system (CNS) or in any other organ, immune and/or inflammation suppression would be beneficial, Parkinson's disease, complication and/or side effects from treatment of Parkinson's disease, AIDS-related dementia complex HIV-related encephalopathy, Devic's disease, Sydenham chorea, Alzheimer's disease and other degenerative diseases, conditions or disorders of the CNS, inflammatory components of stokes, post-polio syndrome, immune and inflammatory components of psychiatric disorders, myelitis, encephalitis, subacute sclerosing pan-encephalitis, encephalomyelitis, acute neuropathy, subacute neuropathy, chronic neuropathy, Guillaim-Barre syndrome, Sydenham chora, myasthenia gravis, pseudo-tumour cerebri, Down's Syndrome, Huntington's disease, amyotrophic lateral sclerosis, inflammatory components of CNS compression or CNS trauma or infections of the CNS, inflammatory components of muscular atrophies and dystrophies, and immune and inflammatory related diseases, conditions or disorders of the central and peripheral nervous systems, post-traumatic inflammation, septic shock, infectious diseases, inflammatory complications or side effects of surgery or organ, inflammatory and/or immune complications and side effects of gene therapy, e.g. due to infection with a viral carrier, or inflammation associated with AIDS, to suppress or inhibit a humoral and/or cellular immune response, to treat or ameliorate monocyte or leukocyte proliferative diseases, e.g. leukaemia, by reducing the amount of monocytes or lymphocytes, for the prevention and/or treatment of graft rejection in cases of transplantation of natural or artificial cells, tissue and organs such as cornea, bone marrow, organs, lenses, pacemakers, natural or artificial skin tissue.

The present invention is also useful in methods for altering the fate of a cell, tissue or organ type by altering Notch pathway function in the cell. Thus, the present application has application in the treatement of malignant and pre-neoplastic disorders. The present invention is especially useful in relation to adenocarcinomas such as: small cell lung cancer, and cancer of the kidney, uterus, prostrate, bladder, ovary, colon and breast. For example, malignancies which may be treatable according to the present invention include acute and chronic leukemias, lymphomas, myelomas, sarcomas such as fibrosarcoma, myxosarcoma, liposarcoma, lymphangioendotheliosarcoma, angiosarcoma, endotheliosarcoma, chondrosarcoma, osteogenic sarcoma, chordoma, lymphangiosarcoma, synovioma, mesothelioma, leimyosarcoma, rhabdomyosarcoma, colon carcinoma, ovarian cancer, prostate cancer, pancreatic cancer, breasy cancer, squamous cell carcinoma, basal cell carcinoma, adenocarcinoma, sewat gland carcinoma, sebaceous gland carcinoma, papillary carcinoma, papillary adenocarcinomas, cystadenocarcinoma, medullary carcinoma, bronchogenic carcinoma, choriocarcinoma, renal cell carcinoma, hepatoma, bile duct carcinoma seminoma, embryonal carcinoma, cervical cancer, testicular tumour, lung carcinoma, small cell lung carcinoma, bladder carcinoma, epithelial carcinoma, glioma, astrocytoma, ependymoma, pinealoma, hemangioblastoma, acoustic neuoma, medulloblastoma, craniopharyngioma, oligodendroglioma, menangioma, melanoma, neutroblastoma and retinoblastoma.

The present invention may also have application in the treatment of nervous system disorders. Nervous system disorders which may be treated according to the present invention include neurological lesions including traumatic lesions resulting from physical injuries; ischaemic lesions; malignant lesions; infectious lesions such as those caused by HIV, herpes zoster or herpes simplex virus, Lyme disease, tuberculosis or syphilis; degenerative lesions and diseases and demyelinated lesions.

The present invention may be used to treat, for example, diabetes (including diabetic neuropathy, Bell's palsy), systemic lupus erythematosus, sarcoidosis, multiple sclerosis, human immunodeficiency virus-associated myelopathy, transverse myelopathy or various etiologies, progressive multifocal leukoencephalopathy, central pontine myelinolysis, Parkinson's disease, Alzheimer's disease, Huntington's chorea, amyotrophic lateral sclerosis, cerebral infarction or ischemia, spinal cord infarction or ischemia, progressive spinal muscular atrophy, progressive bulbar palsy, primary lateral sclerosis, infantile and juvenile muscular atrophy, progressive bulbar paralysis of childhood (Fazio-Londe syndrome), poliomyelitis and the post polio syndrome, and Hereditary Motorsensory Neuropathy (Charcot-Marie-Tooth Disease).

The present invention may further be useful in the promotion of tissue regeneration and repair. The present invention, therefore, may also be used to treat diseases associated with defective tissue repair and regeneration such as, for example, cirrhosis of the liver, hypertrophic scar formation and psoriasis. The invention may also be useful in the treatment of neutropenia or anemia and in techniques of organ regeneration and tissue engineering.

Pharmaceutical Compositions

The present invention provides a pharmaceutical composition comprising administering a therapeutically effective amount of at least one compound identified by the method of the present invention and a pharmaceutically acceptable carrier, diluent or excipients (including combinations thereof).

The pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the compound is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

Administration

Typically, a physician will determine the actual dosage which will be most suitable for an individual subject and it will vary with the age, weight and response of the particular patient. The dosages below are exemplary of the average case. There can, of course, be individual instances where higher or lower dosage ranges are merited.

The compositions of the present invention may be administered by direct injection. The composition may be formulated for parenteral, mucosal, intramuscular, intravenous, subcutaneous, intraocular or transdermal administration.

The term “administered” includes delivery by viral or non-viral techniques. Viral delivery mechanisms include but are not limited to adenoviral vectors, adeno-associated viral (AAV) vectors, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof. The routes for such delivery mechanisms include but are not limited to mucosal, nasal, oral, parenteral, gastrointestinal, topical, or sublingual routes.

The term “administered” includes but is not limited to delivery by a mucosal route, for example, as a nasal spray or aerosol for inhalation or as an ingestable solution; a parenteral route where delivery is by an injectable form, such as, for example, an intravenous, intramuscular, intradermal, intra-articular, intrathecal, intra-peritoneal or subcutaneous route, or via the alimentary tract (for example, via the Peyers patches).

The routes of administration and dosages described are intended only as a guide since a skilled practitioner will be able to determine readily the optimum route of administration and dosage for any particular patient depending on, for example, the age, weight and condition of the patient. Preferably the pharmaceutical compositions are in unit dosage form. The present invention includes both human and veterinary applications.

Antigen Presenting Cells

Where required, antigen-presenting cells (APCs) may be “professional” antigen presenting cells or may be another cell that may be induced to present antigen to T cells. Alternatively an APC precursor may be used which differentiates or is activated under the conditions of culture to produce an APC.

APCs include dendritic cells (DCs) such as interdigitating DCs or follicular DCs, Langerhans cells, PBMCs, macrophages, B-lymphocytes, or other cell types such as epithelial cells, fibroblasts or endothelial cells, activated or engineered by transfection to express a MHC molecule (Class I or II) on their surfaces. Precursors of APCs include CD34⁺ cells, monocytes, fibroblasts and endothelial cells. The APCs or precursors may be modified by the culture conditions or may be genetically modified, for instance by transfection of one or more genes encoding proteins which play a role in antigen presentation and/or in combination of selected cytokine genes which would promote to immune potentiation (for example IL-2, IL-12, IFN-γ, TNF-α, IL-18 etc.). Such proteins include MHC molecules (Class I or Class II), CD80, CD86, or CD40. Most preferably DCs or DC-precursors are included as a source of APCs.

Dendritic cells (DCs) can be isolated/prepared by a number of means, for example they can either be purified directly from peripheral blood, or generated from CD34+precursor cells for example after mobilisation into peripheral blood by treatment with GM-CSF, or directly from bone marrow. From peripheral blood, adherent precursors can be treated with a GM-CSF/IL-4 mixture (Inaba K, et al. (1992) J. Exp. Med. 175: 1157-1167 (Inaba)), or from bone marrow, non-adherent CD34⁺ cells can be treated with GM-CSF and TNF-a (Caux C, et al. (1992) Nature 360: 258-261 (Caux)). DCs can also be routinely prepared from the peripheral blood of human volunteers, similarly to the method of Sallusto and Lanzavecchia (Sallusto F and Lanzavecchia A (1994) J. Exp. Med. 179: 1109-1118) using purified peripheral blood mononucleocytes (PBMCs) and treating 2 hour adherent cells with GM-CSF and IL-4. If required, these may be depleted of CD19⁺ B cells and CD3⁺, CD2⁺ T cells using magnetic beads (Coffin R S, et al. (1998) Gene Therapy 5: 718-722 (Coffin)). Culture conditions may include other cytokines such as GM-CSF or IL-4 for the maintenance and, or activity of the dendritic cells or other antigen presenting cells.

Thus, it will be understood that the term “antigen presenting cell or the like” are used herein is not intended to be limited to APCs. The skilled man will understand that any vehicle capable of presenting to the T cell population may be used, for the sake of convenience the term APCs is used to refer to all these. As indicated above, preferred examples of suitable APCs include dendritic cells, L cells, hybridomas, fibroblasts, lymphomas, macrophages, B cells or synthetic APCs such as lipid membranes.

T Cells

Where required, T cells from any suitable source, such as a healthy patient, may be used and may be obtained from blood or another source (such as lymph nodes, spleen, or bone marrow). They may optionally be enriched or purified by standard procedures. The T cells may be used in combination with other immune cells, obtained from the same or a different individual. Alternatively whole blood may be used or leukocyte enriched blood or purified white blood cells as a source of T cells and other cell types. It is particularly preferred to use helper T cells (CD4⁺). Alternatively other T cells such as CD8⁺ cells may be used. It may also be convenient to use cell lines such as T cell hybridomas.

Exposure of Agent to APCs and T Cells

T cells/APCs may be cultured as described above. For example, they may be prepared for administration to a patient or incubated with T cells in vitro (ex vivo).

Where treated ex-vivo, modified cells of the present invention are preferably administered to a host by direct injection into the lymph nodes of the patient. Typically from 10⁴ to 10⁸ treated cells, preferably from 10⁵ to 10⁷ cells, more preferably about 10⁶ cells are administered to the patient. Preferably, the cells will be taken from an enriched cell population.

As used herein, the term “enriched” as applied to the cell populations of the invention refers to a more homogeneous population of cells which have fewer other cells with which they are naturally associated. An enriched population of cells can be achieved by several methods known in the art. For example, an enriched population of T-cells can be obtained using immunoaffinity chromatography using monoclonal antibodies specific for determinants found only on T-cells.

Enriched populations can also be obtained from mixed cell suspensions by positive selection (collecting only the desired cells) or negative selection (removing the undesirable cells). The technology for capturing specific cells on affinity materials is well known in the art (Wigzel, et al., J. Exp. Med., 128:23, 1969; Mage, et al., J. Immunol. Meth, 15:47, 1977; Wysocki, et al., Proc. Natl. Acad. Sci. U.S.A., 75:2844, 1978; Schrempf-Decker, et al., J. Immunol Meth., 32:285, 1980; Muller-Sieburg, et al., Cell, 44:653, 1986).

Monoclonal antibodies against antigens specific for mature, differentiated cells have been used in a variety of negative selection strategies to remove undesired cells, for example, to deplete T-cells or malignant cells from allogeneic or autologous marrow grafts, respectively (Gee, et al., J.N.C.I. 80:154, 1988). Purification of human hematopoietic cells by negative selection with monoclonal antibodies and immunomagnetic microspheres can be accomplished using multiple monoclonal antibodies (Griffin, et al., Blood, 63:904, 1984).

Procedures for separation of cells may include magnetic separation, using antibody coated magnetic beads, affinity chromatography, cytotoxic agents joined to a monoclonal antibody or used in conjunction with a monoclonal antibody, for example, complement and cytotoxins, and “panning” with antibodies attached to a solid matrix, for example, plate, or other convenient technique. Techniques providing accurate separation include fluorescence activated cell sorters, which can have varying degrees of sophistication, for example, a plurality of color channels, low angle and obtuse light scattering detecting channels, impedance channels, etc.

It will be appreciated that in one embodiment the therapeutic agents used in the present invention may be administered directly to patients in vivo. Alternatively or in addition, the agents may be administered to cells such as T cells and/or APCs in an ex vivo manner. For example, leukocytes such as T cells or APCs may be obtained from a patient or donor in known manner, treated/incubated ex vivo in the manner of the present invention, and then administered to a patient. In addition, it will be appreciated that a combination of routes of administration may be employed if desired. For example, where appropriate one component (such as the modulator of Notch signalling) may be administered ex-vivo and the other may be administered in vivo, or vice versa.

Introduction of Nucleic Acid Sequences into APCs and T-Cells

T-cells and APCs as described above are cultured in a suitable culture medium such as DMEM or other defined media, optionally in the presence of fetal calf serum.

Polypeptide substances may be administered to T-cells and/or APCs by introducing nucleic acid constructs/viral vectors encoding the polypeptide into cells under conditions that allow for expression of the polypeptide in the T-cell and/or APC. Similarly, nucleic acid constructs encoding antisense constructs may be introduced into the T-cells and/or APCs by transfection, viral infection or viral transduction.

In a preferred embodiment, nucleotide sequences will be operably linked to control sequences, including promoters/enhancers and other expression regulation signals. The term “operably linked” means that the components described are in a relationship permitting them to function in their intended manner. A regulatory sequence “operably linked” to a coding sequence is peferably ligated in such a way that expression of the coding sequence is achieved under condition compatible with the control sequences.

The promoter is typically selected from promoters which are functional in mammalian cells, although prokaryotic promoters and promoters functional in other eukaryotic cells may be used. The promoter is typically derived from promoter sequences of viral or eukaryotic genes. For example, it may be a promoter derived from the genome of a cell in which expression is to occur. With respect to eukaryotic promoters, they may be promoters that function in a ubiquitous manner (such as promoters of a-actin, b-actin, tubulin) or, alternatively, a tissue-specific manner (such as promoters of the genes for pyruvate kinase). Tissue-specific promoters specific for lymphocytes, dendritic cells, skin, brain cells and epithelial cells within the eye are particularly preferred, for example the CD2, CD11c, keratin 14, Wnt-1 and Rhodopsin promoters respectively. Preferably the epithelial cell promoter SPC is used. They may also be promoters that respond to specific stimuli, for example promoters that bind steroid hormone receptors. Viral promoters may also be used, for example the Moloney murine leukaemia virus long terminal repeat (MMLV LTR) promoter, the rous sarcoma virus (RSV) LTR promoter or the human cytomegalovirus (CMV) IE promoter.

It may also be advantageous for the promoters to be inducible so that the levels of expression of the heterologous gene can be regulated during the life-time of the cell. Inducible means that the levels of expression obtained using the promoter can be regulated.

Any of the above promoters may be modified by the addition of further regulatory sequences, for example enhancer sequences. Chimeric promoters may also be used comprising sequence elements from two or more different promoters.

Alternatively (or in addition), the regulatory sequences may be cell specific such that the gene of interest is only expressed in cells of use in the present invention. Such cells include, for example, APCs and T-cells.

If required, a small aliquot of cells may be tested for up-regulation of Notch signalling activity as described above. The cells may be prepared for administration to a patient or incubated with T-cells in vitro (ex vivo).

Tolerisation Assays

Any of the assays described above (see “Assays”) can be adapted to monitor or to detect reduced reactivity and tolerisation in immune cells for use in clinical applications. Such assays will involve, for example, detecting increased Notch-ligand expression or activity in host cells or monitoring Notch cleavage in donor cells. Further methods of monitoring immune cell activity are set out below.

Immune cell activity may be monitored by any suitable method known to those skilled in the art. For example, cytotoxic activity may be monitored. Natural killer (NK) cells will demonstrate enhanced cytotoxic activity after activation. Therefore any drop in or stabilisation of cytotoxicity will be an indication of reduced reactivity.

Once activated, leukocytes express a variety of new cell surface antigens. NK cells, for example, will express transferrin receptor, HLA-DR and the CD25 IL-2 receptor after activation. Reduced reactivity may therefore be assayed by monitoring expression of these antigens.

Hara et al. Human T-cell Activation: III, Rapid Induction of a Phosphorylated 28 kD/32 kD Disulfide linked Early Activation Antigen (EA-1) by 12-O-tetradecanoyl Phorbol-13-Acetate, Mitogens and Antigens, J. Exp. Med., 164:1988 (1986), and Cosulich et al. Functional Characterization of an Antigen (MLR3) Involved in an Early Step of T-Cell Activation, PNAS, 84:4205 (1987), have described cell surface antigens that are expressed on T-cells shortly after activation. These antigens, EA-1 and MLR3 respectively, are glycoproteins having major components of 28 kD and 32 kD. EA-1 and MLR3 are not HLA class II antigens and an MLR3 Mab will block IL-1 binding. These antigens appear on activated T-cells within 18 hours and can therefore be used to monitor immune cell reactivity.

Additionally, leukocyte reactivity may be monitored as described in EP 0325489, which is incorporated herein by reference. Briefly this is accomplished using a monoclonal antibody (“Anti-Leu23”) which interacts with a cellular antigen recognised by the monoclonal antibody produced by the hybridoma designated as ATCC No. HB-9627.

Anti-Leu 23 recognises a cell surface antigen on activated and antigen stimulated leukocytes. On activated NK cells, the antigen, Leu 23, is expressed within 4 hours after activation and continues to be expressed as late as 72 hours after activation. Leu 23 is a disulfide-linked homodimer composed of 24 kD subunits with at least two N-linked carbohydrates.

Because the appearance of Leu 23 on NK cells correlates with the development of cytotoxicity and because the appearance of Leu 23 on certain T-cells correlates with stimulation of the T-cell antigen receptor complex, Anti-Leu 23 is useful in monitoring the reactivity of leukocytes.

Further details of techniques for the monitoring of immune cell reactivity may be found in: ‘The Natural Killer Cell’ Lewis C. E. and J. O'D. McGee 1992. Oxford University Press; Trinchieri G. ‘Biology of Natural Killer Cells’ Adv. Immunol. 1989 vol 47 pp 187-376; ‘Cytokines of the Immune Response’ Chapter 7 in “Handbook of Immune Response Genes”. Mak T. W. and J. J. L. Simard 1998, which are incorporated herein by reference.

Preparation of Primed APCs and Lymphocytes

According to one aspect of the invention immune cells may be used to present antigens or allergens and/or may be treated to modulate expression or interaction of Notch, a Notch ligand or the Notch signalling pathway. Thus, for example, Antigen Presenting Cells (APCs) may be cultured in a suitable culture medium such as DMEM or other defined media, optionally in the presence of a serum such as fetal calf serum. Optimum cytokine concentrations may be determined by titration. One or more modulators of Notch signalling are then typically added to the culture medium together with the antigen of interest. The antigen may be added before, after or at substantially the same time as the substance(s). Cells are typically incubated with the substance(s) and antigen for at least one hour, preferably at least 3 hours, suitably at least 9, 12, 24, 48 or 36 or more hours at 37° C. If required, a small aliquot of cells may be tested for modulated target gene expression as described above. Alternatively, cell activity may be measured by the inhibition of T cell activation by monitoring surface markers, cytokine secretion or proliferation as described in WO98/20142.

As discussed above, polypeptide substances may in one embodiment be administered to APCs by introducing nucleic acid constructs/viral vectors encoding the polypeptide into cells under conditions that allow for expression of the polypeptide in the APC. Similarly, nucleic acid constructs encoding antigens may be introduced into the APCs by transfection, viral infection or viral transduction.

Preparation of Regulatory T Cells (and B Cells) Ex Vivo

The techniques described below are described in relation to T cells, but are equally applicable to B cells. The techniques employed are essentially identical to that described for APCs alone except that T cells are generally co-cultured with the APCs. However, it may be preferred to prepare primed APCs first and then incubate them with T cells. For example, once the primed APCs have been prepared, they may be pelleted and washed with PBS before being resuspended in fresh culture medium. This has the advantage that if, for example, it is desired to treat the T cells with a different substance(s), then the T cell will not be brought into contact with the different substance(s) used with the APC. Once primed APCs have been prepared, it is not always necessary to administer any substances to the T cell since the primed APC is itself capable of inducing immunotolerance leading to increased Notch or Notch ligand expression in the T cell, presumably via Notch/Notch ligand interactions between the primed APC and T cell.

Incubations will typically be for at least 1 hour, preferably at least 3, 6, 12, 24, 48 or 36 or more hours, in suitable culture medium at 37° C. The progress of Notch signalling may be determined for a small aliquot of cells using the methods described above. T cells transfected with a nucleic acid construct directing the expression of, for example Delta, may be used as a control. Induction of immunotolerance may be determined, for example, by subsequently challenging T cells with antigen and measuring IL-2 production compared with control cells not exposed to APCs.

Primed T cells or B cells may also be used to induce immunotolerance in other T cells or B cells in the absence of APCs using similar culture techniques and incubation times.

Alternatively, T cells may be cultured and primed in the absence of APCs by use of APC substitutes such as anti-TCR antibodies (e.g. anti-CD3) with or without antibodies to costimulatory molecules (e.g. anti-CD28) or alternatively T cells may be activated with MHC-peptide complexes (e.g. tetramers).

Induction of immunotolerance may be determined by subsequently challenging T cells with antigen and measuring IL-2 production compared with control cells not exposed to APCs.

T cells or B cells which have been primed in this way may be used according to the invention to promote or increase immunotolerance in other T cells or B cells.

The present invention will now further be described with reference to the following non-limiting Examples:

### EXAMPLES

### Example 1

### CD4+ Cell Purification

Spleens were removed from mice (variously Balb/c females, 8-10 weeks, C57B/6 females, 8-10 weeks, CARD1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

### Example 2

### Antibody Coating

The following protocols were used for coating 96 well flat-bottomed plates with antibodies.

A) The plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 10011 DPBS plus 10 μg/ml Notch ligand (mouse Delta 1 extracellular domain/Ig4Fc fusion protein; Fc-delta).

The plates were incubated for 2-3 hours at 37° C. then washed again with DPBS before cells (prepared as in Example 1) were added.

B) Alternatively, the plates were coated with DPBS plus 1 μg/ml anti-hamsterIgG antibody (Pharmingen Cat No 554007) plus 1 μg/ml anti-IgG4 antibody. 100 μl of coating mixture was added per well. Plates were incubated overnight at 4° C. then washed with DPBS. Each well then received either 100 μl DPBS plus anti-CD3 antibody (1 μg/ml) or, 100 μl DPBS plus anti-CD3 antibody (1 μg/ml) plus Fc-delta (10 μg/ml). The plates were incubated for 2-3 hours at 37° C. then washed again with DPBS before cells (prepared as in Example 1) were added.

### Example 3

### Primary Polyclonal Stimulation

CD4+ cells were cultured in 96 well, flat-bottomed plates pre-coated according to Example 2 (A) or 2 (B). Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of R10F medium was then added to each well to give a final volume of 200 μl (2×10⁵ cells/well, anti-CD28 final concentration 2 μg/ml) The plates were then incubated at 37° C. for 72 hours.

125 μl supernatant was then removed from each well and stored at −20° C. until tested by ELISA for IL-10, IFNg and IL-13 using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

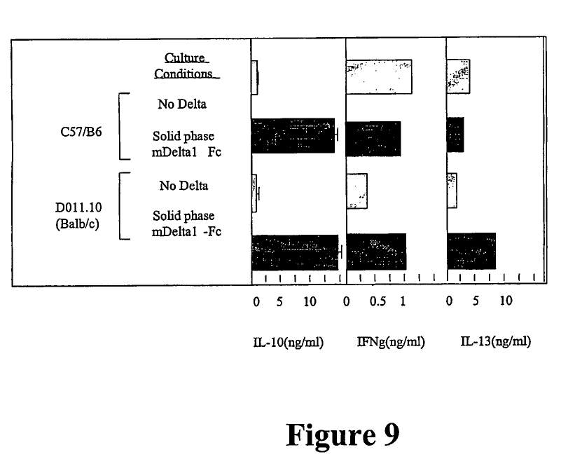

Results are shown in FIG. 9.

### Example 4

### Real Time PCR Analysis of Primary Stimulated CD4+ Cells

Murine (Balb/c) stimulated CD4⁺ T-cells from Example 3 were harvested at 4, 16 and 24 hours. Total cellular RNA was isolated using the RNeasy™ RNA isolation kit (Qiagen, Crawley, UK) according to the manufacturer's guidelines.

In each case 1 μg of total RNA was reverse transcribed using SuperScript™ II Reverse Transcriptase (Invitrogen, Paisley, UK) using Oligo dT₍₁₂₋₁₈₎ or a random decamer mix according to the manufacturer's guidelines. After synthesis, Oligo dT₍₁₂₋₁₈₎- and random decamer-primed cDNAs were mixed in equal proportions to provide the working cDNA sample for real-time quantitative PCR analysis.

Real-time quantitative PCR was performed using the Roche Lightcycler™ system (Roche, UK) and SYBR green detection chemistry according to the manufacturer's guidelines. The following HPLC-purified primer pairs were used for cDNA-specific amplification (5′ to 3′):

mouse 18s rRNA:

| | | |
|---|---|---|
| Forward | GTAACCCGTTGAACCCCATT | (SEQ ID NO: 27) |
| Reverse | CCATCCAATCGGTAGTAGCG | (SEQ ID NO: 28) |

mouse Hes-1:

| | | |
|---|---|---|
| Forward | GGTGCTGATAACAGCGGAAT | (SEQ ID NO: 29) |
| Reverse | ATTTTTGGAATCCTTCACGC | (SEQ ID NO: 30) |

The endpoint used in real-time PCR quantification, the Crossing Point (Cp), is defined as the PCR cycle number that crosses an algorithm-defined signal threshold. Quantitative analysis of gene-specific cDNA was achieved firstly by generating a set of standards using the Cps from a set of serially-diluted gene-specific amplicons which had been previously cloned into a plasmid vector (pCR2.1, Invitrogen). These serial dilutions fall into a standard curve against which the Cₚs from the cDNA samples were compared. Using this system, expression levels of the 18S rRNA housekeeping gene were generated for each cDNA sample. Hes-1 was then analysed by the same method using serially-diluted Hes-1-specific standards, and the Hes-1 value divided by the 18S rRNA value to generate a value, which represents the relative expression of Hes-1 in each cDNA sample. All Cp analysis was performed using the Second Derivative Maximum algorithm within the Lightcycler system software.

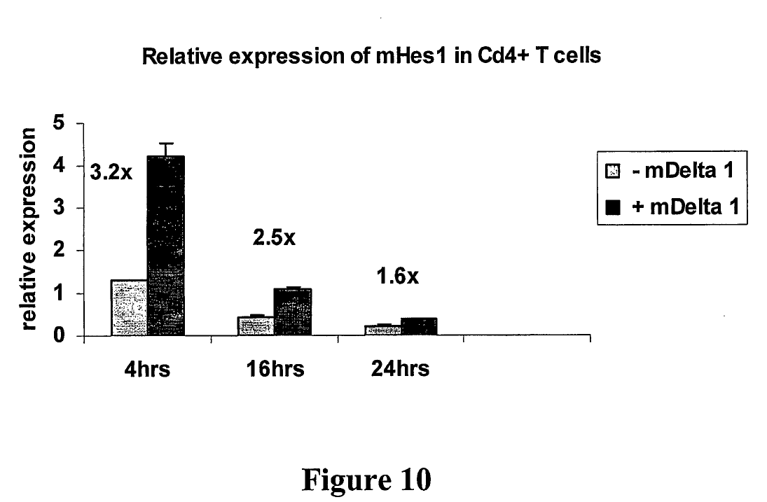

Results (HES-1 expression relative to 18S rRNA expression with and without Fc-delta) are shown in FIG. 10.

### Example 5

### Screening Under Polarising Conditions

Plates were coated and CD4+ cells added as in Example 2 (A).

The procedure of Example 3 was then followed, except that instead of adding 100 μl R10F medium per well as in Example 3, 100 μl of polarising cocktail was added per well as follows:

Un-polarised cells: R10F medium.

Th1 polarised cells: R10F medium plus anti-IL-4 antibody (10 μg/ml, Pharmingen Cat No 554432) plus IL-12 (10 ng/ml, Peprotech 210-12).

Th2 polarised cells: R10Fmedium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

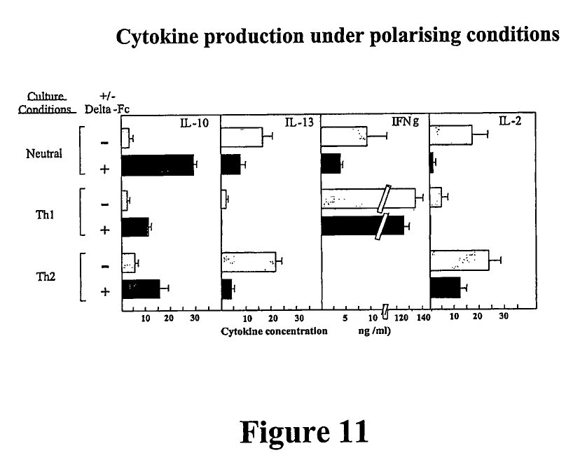

Cells were then stimulated and cytokines (IL-10, IFNγ and IL-13) measured by ELISA as described in Example 3. Results are shown in FIG. 11.

### Example 6

### Soluble Ligand

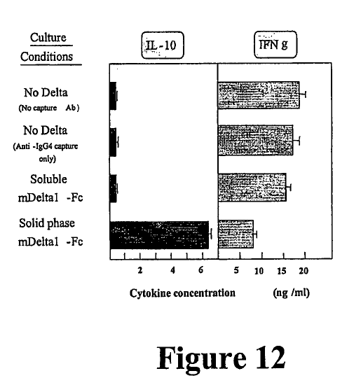

The procedure of Example 2(A), with the modification that ligand was not added to the plate, and Example 3, with the modification that soluble Fc-delta was added with the R10F medium, was used to compare soluble Fc-delta with plate-bound Fc-delta against controls. Results are shown in FIG. 12.

### Example 7

### Secondary Stimulation

7 days after primary stimulation all cells were harvested and counted then stimulated in one of three ways as follows:

Re-Stimulation

Cells were re-stimulated exactly as for primary stimulation (Example 3).

Re-Challenge on Anti-CD3/CD28

96-well flat-bottomed plates were coated with PBS plus 1 μg/ml anti-CD3 antibody. The plates were incubated overnight at 4° C. then washed with DPBS.

The cells were re-suspended at 2×10⁶/ml in R10F medium plus anti-CD28 antibody (4 μg/ml). 100 μl cell suspension was added per well. 100 μl of R10F medium was then added per well to give a final volume of 200 μl. (2×10⁵ cells/well, anti-CD28 final concentration 2 μg/ml). The plates were then incubated at 37° C. for 72 hours. After 72 hours supernatants were removed for ELISA as described in Example 3 (primary stimulation).

Re-Stimulation with APC Plus Anti-CD3

Primary stimulated cells from Example 3 were harvested after 7 days and restimulated with APCs of the same strain (2×10⁴ per well) plus anti-CD3 antibody.

Mouse spleen cells were isolated as described in Example 1 up to the counting step. Thy-1.2 antibody-binding cells were then removed on a MACS column and the flowthrough was recovered and treated with mitomycin-C for 45 minutes then added to a 96 well plate in 100 μl R10F medium with equal numbers of cells from Example 3 and 0.5 μg/ml anti-CD3 antibody.

Cell proliferation was measured using a kit from Roche Molecular Biochemicals, Cell Proliferation ELISA, BrdU (chemiluminescent) 1 669 915, according to the manufacturer's instructions. Plates were pulsed at 72 hours and read on a luminometer.

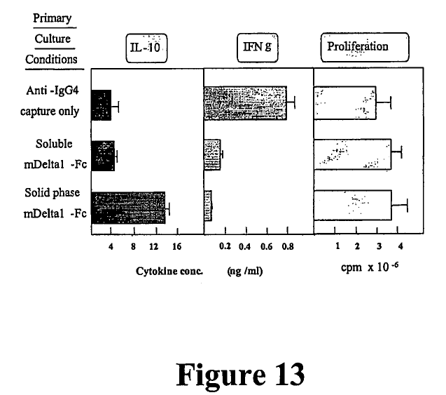

Cytokines (IL-10 and IFN-γ) were measured as described in Example 3. Results are shown in FIG. 13.

### Example 8

### CHO-N2 (N27) Luciferase Reporter Assay

A) Construction of Luciferase Reporter Plasmid 10×CBF1-Luc (pLOR91)

An adenovirus major late promoter TATA-box motif with BglII and HindIII cohesive ends was generated as follows:

| | |
|---|---|
| BglII HindIII | |
| GATCTGGGGGGCTATAAAAGGGGGTA | (SEQ ID NO: 31) |
| ACCCCCCGATATTTTCCCCCATTCGA | (SEQ ID NO: 32) |

This was cloned into plasmid pGL3-Basic (Promega) between the BgiII and HindIII sites to generate plasmid pGL3-AdTATA.

A TP1 promoter sequence (TP1; equivalent to 2 CBF1 repeats) with BamH1 and BglII cohesive ends was generated as follows:

| | |
|---|---|
| BamH1 BglII | |
| 5′ GATCCCGACTCGTGGGAAAATGGGCGGAAGGGCACCGTGGGAAAATAGTA 3′ | (SEQ ID NO: 33) |
| 3′ GGCTGAGCACCCTTTTACCGGCCTTCCCGTGGCACCCTTTTATCATCTAG 5′ | (SEQ ID NO: 34) |

This sequence was pentamerised by repeated insertion into a BglII site and the resulting TP1 pentamer (equivalent to 10 CBF1 repeats) was inserted into pGL3-AdTATA at the BglII site to generate plasmid pLOR91.

B) Generation of Stable CHO Reporter Cell Line Expressing Full Length Notch2 and 10×CBF1-Luc Reporter Cassette

A cDNA clone spanning the complete coding sequence of the human Notch2 gene (see, e.g. GenBank Accession No AF315356) was constructed as follows. A 3′ cDNA fragment encoding the entire intracellular domain and a portion of the extracellular domain was isolated from a human placental cDNA library (OriGene Technologies Ltd., USA) using a PCR-based screening strategy. The remaining 5′ coding sequence was isolated using a RACE (Rapid Amplification of cDNA Ends) strategy and ligated onto the existing 3′ fragment using a unique restriction site common to both fragments (Cla I). The resulting full-length cDNA was then cloned into the mammalian expression vector pcDNA3.1-V5-HisA (Invitrogen) without a stop codon to generate plasmid pLOR92. When expressed in mammalian cells, pLOR92 thus expresses the full-length human Notch2 protein with V5 and His tags at the 3′ end of the intracellular domain.

Wild-type CHO-K1 cells (e.g., see ATCC No CCL 61) were transfected with pLOR92 (pcDNA3.1-FLNotch2-V5-His) using Lipfectamine 2000™ (Invitrogen) to generate a stable CHO cell clone expressing full length human Notch2 (N2). Transfectant clones were selected in Dulbecco's Modified Eagle Medium (DMEM) plus 10% heat inactivated fetal calf serum ((HI)FCS) plus glutamine plus Penicillin-Streptomycin (P/S) plus 1 mg/ml G418 (Geneticin™—Invitrogen) in 96-well plates using limiting dilution. Individual colonies were expanded in DMEM plus 10% (HI)FCS plus glutamine plus P/S plus 0.5 mg/ml G418. Clones were tested for expression of N2 by Western blots of cell lysates using an anti-V5 monoclonal antibody (Invitrogen). Positive clones were then tested by transient transfection with the reporter vector pLOR91 (10×CBF1-Luc) and co-culture with a stable CHO cell clone (CHO-Delta) expressing full length human delta-like ligand 1 (DLL1; e.g., see GenBank Accession No AF196571). (CHO-Delta was prepared in the same way as the CHO Notch 2 clone, but with human DLL1 used in place of Notch 2. A strongly positive clone was selected by Western blots of cell lysates with anti-V5 mAb.)

One CHO-N2 stable clone, N27, was found to give high levels of induction when transiently transfected with pLOR91 (10×CBF 1-Luc) and co-cultured with the stable CHO cell clone expressing full length human DLL1 (CHO-Delta1). A hygromycin gene cassette (obtainable from pcDNA3.1/hygro, Invitrogen) was inserted into pLOR91 (10×CBF1-Luc) using BamHI and Sal1 and this vector (10×CBF1-Luc-hygro) was transfected into the CHO-N2 stable clone (N27) using Lipfectamine 2000 (Invitrogen). Transfectant clones were selected in DMEM plus 10% (HI)FCS plus glutamine plus P/S plus 0.4 mg/ml hygromycin B (Invitrogen) plus 0.5 mg/ml G418 (Invitrogen) in 96-well plates using limiting dilution. Individual colonies were expanded in DMEM plus 10% (HI)FCS plus glutamine plus P/S+0.2 mg/ml hygromycin B plus 0.5 mg/ml G418 (Invitrogen).

Clones were tested by co-culture with a stable CHO cell clone expressing FL human DLL1. Three stable reporter cell lines were produced N27#11, N27#17 and N27#36. N27#11 was selected for further use because of its low background signal in the absence of Notch signalling, and hence high fold induction when signalling is initiated. Assays were set up in 96-well plates with 2×10⁴ N27#11 cells per well in 100 μl per well of DMEM plus 10% (HI)FCS plus glutamine plus P/S.

C) Transient Transfection of CHO-N2 Cells with 10×CBF1-Luc

Alternatively, for transient transfection, CHO-N2 (Clone N27) cells were maintained in DMEM plus 10% (HI)FCS plus glutamine plus P/S plus 0.5 mg/ml G418 and a T₈₀ flask of the CHO-N2 cells was transfected as follows. The medium on the cells was replaced with 8 ml of fresh in DMEM plus 10% (HI)FCS plus glutamine plus P/S. In a sterile bijou 10 μg of pLOR91 (10×CBF1-Luc) was added to OptiMem (Invitrogen) to give a final volume of 1 ml and mixed. In a second sterile bijou 20 μl of Lipofectamine 2000 reagent was added to 980 μl of OptiMem and mixed.

The contents of each bijou were mixed and left at room temperature for 20 minutes.

The 2 ml of transfection mixture was added to the flask of cells containing 8 ml of medium and the resulting mixture was left in a CO₂ incubator overnight before removing the transfected cells and adding to the 96-well plate containing the immobilised Notch ligand protein.

The following day the transfected CHO-N2 cells were removed using 0.02% EDTA solution (Sigma), spun down and resuspended in 10 ml DMEM plus 10% (HI)FCS plus glutamine plus P/S. 10 μl of cells were counted and the cell density was adjusted to 2.0×10⁵ cells/ml with fresh DMEM plus 10% (HI)FCS plus glutamine plus P/S. 100 μl per well was added to a 96-well tissue culture plate (flat bottom), i.e. 2.0×10⁴ transfected cells per well, using a multi-channel pipette and the plate was then incubated overnight.

D) Immobilisation of Notch Ligand Protein Directly onto a 96-Well Tissue Culture Plate

10 μg of purified Notch ligand protein was added to sterile PBS in a sterile Eppendorf tube to give a final volume of 1 ml. Serial 1:2 dilutions were made by adding 500 μl into sterile Eppendorf tubes containing 500 μl of sterile PBS to generate dilutions of 10 μg/ml, 5 μg/ml, 2.5 μg/ml, 1.25 μg/ml, 0.625 μg/ml and 0 μg/ml.

The lid of the plate was sealed with parafilm and the plate was left at 4° C. overnight or at 37° C. for 2 hours. The protein was then removed and the plate was washed with 200 μl of PBS.

E) A20-Delta Cells

The IVS, IRES, Neo and pA elements were removed from plasmid pIRESneo2 (Clontech, USA) and inserted into a pUC cloning vector downstream of a chicken beta-actin promoter (e.g., see GenBank Accession No E02199). Mouse Delta-1 (e.g., see GenBank Accession No NM_{—}007865) was inserted between the actin promoter and IVS elements and a sequence with multiple stop codons in all three reading frames was inserted between the Delta and IVS elements.

The resulting construct was transfected into A20 cells using electroporation and G418 to provide A20 cells expressing mouse Delta1 on their surfaces (A20-Delta).

F) CHO and CHO-hDelta1-V5-His Assay Control

CHO cells were maintained in DMEM plus 10% (HI)FCS plus glutamine plus P/S and CHO-hDelta1-V5-His (clone#10) cells were maintained in DMEM plus 10% (HI)FCS plus glutamine plus P/S plus 0.5 mg/ml G418.

Cells were removed using 0.02% EDTA solution (Sigma), spun down and resuspended in 10 ml DMEM plus 10% (HI)FCS plus glutamine plus P/S. 10 μl of cells were counted and the cell density was adjusted to 5.0×10⁵ cells/ml with fresh DMEM plus 10% (HI)FCS plus glutamine plus P/S. 300 μl of each cell line at 5.0×10⁵ cells/ml was added into duplicate wells of a 96-well tissue culture plate. 150 μl of DMEM plus 10% (HI)FCS plus glutamine plus P/S was added in to the next 5 wells below each well. 150 μl of cells were serially diluted into the next 4 wells giving cell density dilution of 5.0×10⁵ cells/ml, 2.5×10⁵ cells/ml, 1.25×10⁵ cells/ml, 0.625×10⁵ cells/ml, 0.3125×10⁵ cells/ml and 0 cells/ml.

100 μl from each well was added into the 96-well plate containing 100 μl of CHO-N2 cells transfected with 10×CBF1-Luc (2.0×10⁴ transfected CHO-N2 cells/well) and the plate was left in an incubator overnight.

G) Cell Co-Culture

5×10⁴ CHO-N2 cells were plated on a 96 well plate. CHO-Delta or A20-Delta cells were titrated in as required (max ratio CHO-N2: CHO-Delta was 1:1, max ratio CHO-N2: A20-Delta was 1:2). The mixture was incubated overnight before conducting a luciferase assay.

H) Luciferase Assay

Supernatant was removed from all wells. 100 μl of PBS and 100 μl of SteadyGlo™ luciferase assay reagent (Promega) was added and the cells were left at room temperature for 5 minutes. The mixture was pipetted up and down 2 times to ensure cell lysis and contents from each well were transferred into a white 96-well OptiPlate™ (Packard). Luminescence was measured in a TopCount™ counter (Packard).

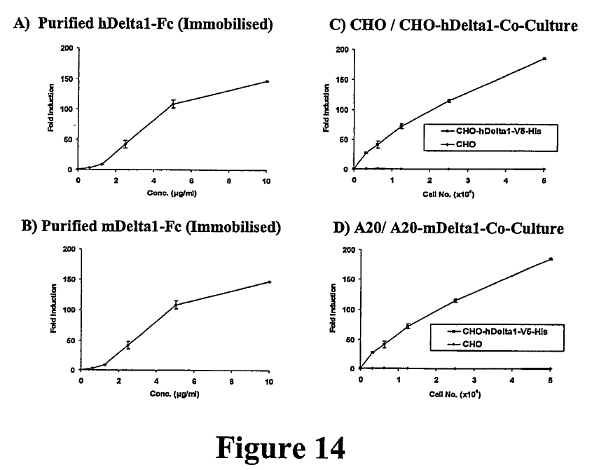

Results of sample assays (using the stable CHO-Notch2-10×CBF1-Luc reporter cell line described above with (A) plate-immobilised human Delta-1/Ig4Fc fusion protein, (B) plate-immobilised mouse Delta-1/Ig4Fc fusion protein, (C)CHO/CHO-human Delta1 co-cultured cells and (D) A20/A20-mouse Delta1 co-cultured cells as actives against corresponding controls) are shown in FIGS. 14A to D.

### Example 9

### Dynabeads Luciferase Assay Method for Detecting Notch Ligand Activity

Fc-tagged Notch ligands were immobilised on Streptavidin-Dynabeads (CELLection Biotin Binder Dynabeads [Cat. No. 115.21] at 4.0×10⁸ beads/ml from Dynal (UK) Ltd; beads) in combination with biotinylated α-IgG-4 (clone JDC14 at 0.5 mg/ml from Pharmingen [Cat. No. 555879]) as follows:

2.5×10⁷ beads (62.5 μl of beads at 4.0×10⁸ beads/ml) and 5 μg biotinylated α-IgG-4 was used for each sample assayed. PBS was added to the beads to 1 ml and the mixture was spun down at 13,000 rpm for 1 minute. Following washing with a further 1 ml of PBS the mixture was spun down again. The beads were then resuspended in a final volume of 100 μl of PBS containing the biotinylated α-IgG-4 in a sterile Eppendorf tube and placed on shaker at room temperature for 30 minutes. PBS to was added to 1 ml and the mixture was spun down at 13,000 rpm for 1 minute and then washed twice more with 1 ml of PBS.

The mixture was then spun down at 13,000 rpm for 1 minute and the beads were resupsended in a 50 μl PBS per sample. 50 μl of biotinylated α-IgG-4-coated beads were added to each sample and the mixture was incubated on a rotary shaker at 4° C. overnight. The tube was then spun at 1000 rpm for 5 minutes at room temperature.

The beads then were washed with 10 ml of PBS, spun down, resupended in 1 ml of PBS, transferred to a sterile Eppendorf tube, washed with a further 2×1 ml of PBS, spun down and resuspended beads in a final volume of 250 μl of DMEM plus 10% (HI)FCS plus glutamine plus P/S, i.e. at 1.0×10⁵ beads/μl.

Stable N27#11 cells from Example 8 (T₈₀ flask) were removed using 0.02% EDTA solution (Sigma), spun down and resuspended in 10 ml DMEM plus 10% (HI)FCS plus glutamine plus P/S. 10 μl of cells were counted and the cell density was adjusted to 1.0×10⁵ cells/ml with fresh DMEM plus 10% (HI)FCS plus glutamine plus P/S. 1.0×10⁵ of the cells were plated out per well of a 24-well plate in a 1 ml volume of DMEM plus 10% (HI)FCS plus glutamine plus P/S and cells were placed in an incubator to settle down for at least 30 minutes.

100 μl of beads were then added in duplicate to the first pair of wells to give 1.0×10⁷ beads/well (100 beads/cell); 20 μl of beads added in duplicate to the second pair of wells to give 2.0×10⁶ beads/well (20 beads/cell); 4 μl of beads added in duplicate to the third pair of wells to give 4.0×10⁵ beads/well (4 beads/cell) and 0 μl of beads added to the fourth pair of wells. The plate was left in a CO₂ incubator overnight.

Luciferase Assay

Supernatant was then removed from all the wells, 150 μl of PBS and 150 μl of SteadyGlo luciferase assay reagent (Promega) were added and the resulting mixture left at room temperature for 5 minutes.

The mixture was then pipetted up and down 2 times to ensure cell lysis and the contents from each well were transferred into an Eppendorf tube, spun at 13,000 rpm for 1 minute and the cleared supernatant was transferred to a white 96-well OptiPlate™ (Packard), leaving the bead pellet behind. Luminescence was then read in a TopCount™ (Packard) counter.

### Example 10

### Dynabeads ELISA Assay Method for Detecting Notch Ligand Activity

M450 Streptavidin Dynabeads were coated with anti-hamster-IgG1 biotinylated monoclonal antibody, anti-human-IgG4 biotinylated monoclonal antibody or both antibodies and rotated for 2 hours at room temperature.

Beads were washed three times with PBS (1 ml). The anti-hamster-IgG1 beads were then further incubated with anti-CD3ε chain monoclonal antibody, the anti-human-IgG4 beads were further incubated with Fc-Delta, and the double coated beads incubated with both anti-CD3ε chain monoclonal antibody and Fc-Delta. Beads were rotated overnight at 4° C., washed three times with PBS (1 ml) and resuspended.

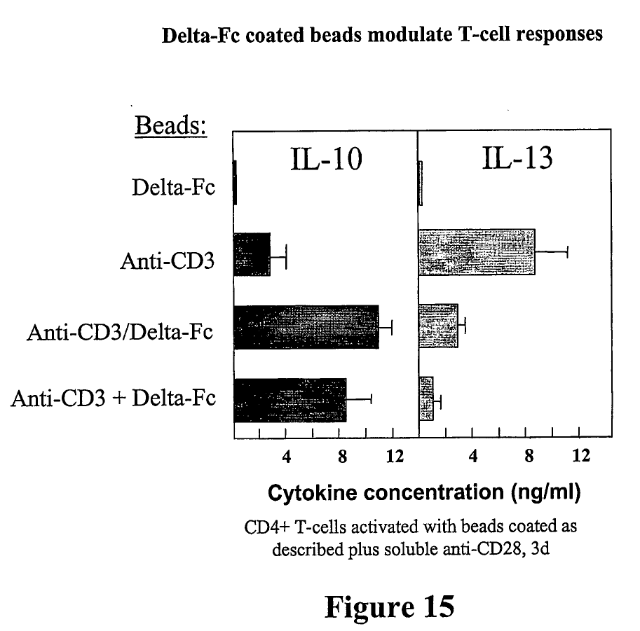

T-cell assays were carried out with CD4+ T-cells and the beads. Supernatants were removed after 72 hours and cytokines measured by ELISA as described in Example 3. Results are shown in FIG. 15.

### Example 11

### Modulation of Cytokine Production by Human CD4+ T Cells in the Presence of Delta1-hIG4 Immobilised on Dynal Microbeads

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 105 CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell) or plate bound anti-CD3 (clone UCHT1, BD Biosciences, 5 μg/ml) and soluble anti-CD28 (clone CD28.2, BD Biosciences, 2 μg/ml). Beads coated with mouse Delta1EC domain-hIgG4 fusion protein (prepared as described above with the modifications that incubation with human IgG4 was for 30-40 minutes at room temperature and incubation with Delta-Fc was for two hours at room temperature) or control beads were added in some of the wells at a 10:1 ratio (beads/cell). The supernatants were removed after 3 or 4 days of incubation at 37° C./5% CO₂/humidified atmosphere and cytokine production was evaluated by ELISA using Pharmingen kits OptEIA Set human IL10 (catalog No. 555157), OptEIA Set human IL-5 (catalog No. 555202) and OptEIA Set human IFNg (catalog No 555142) for IL-10, IL-5 and IFNg respectively and a human TNFa DuoSet from R&D Systems (catalog. No. DY210) for TNFa according to the manufacturer's instructions.

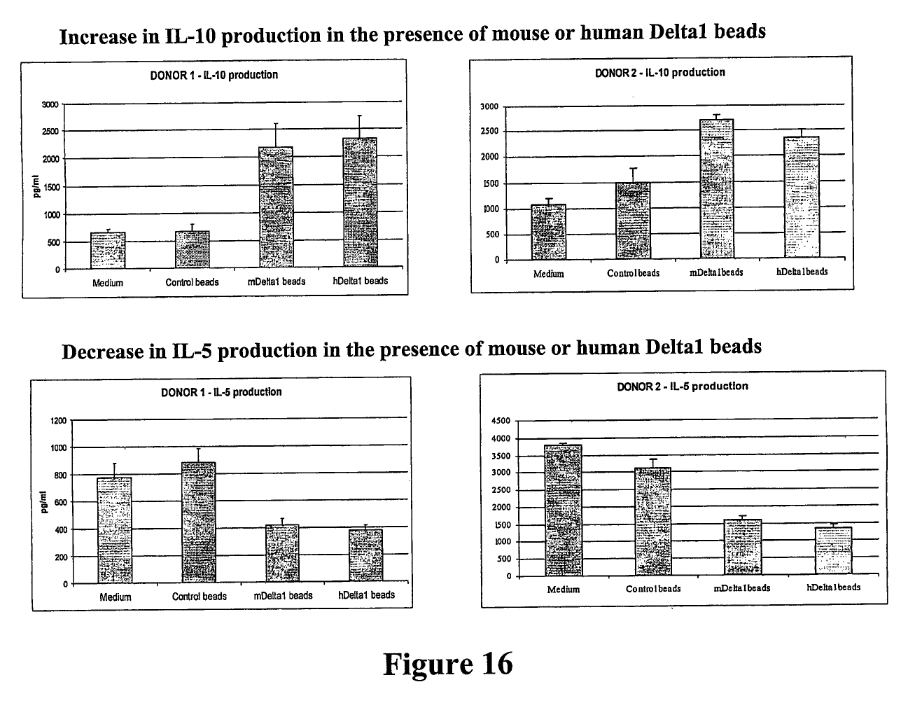

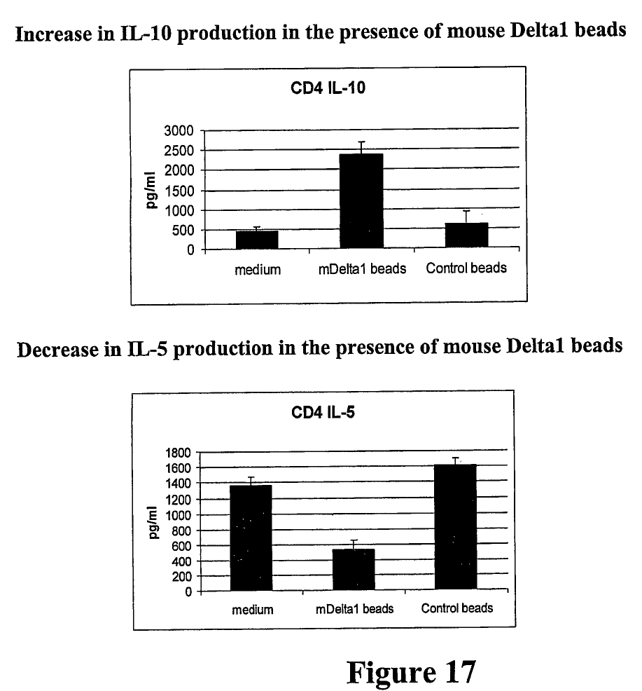

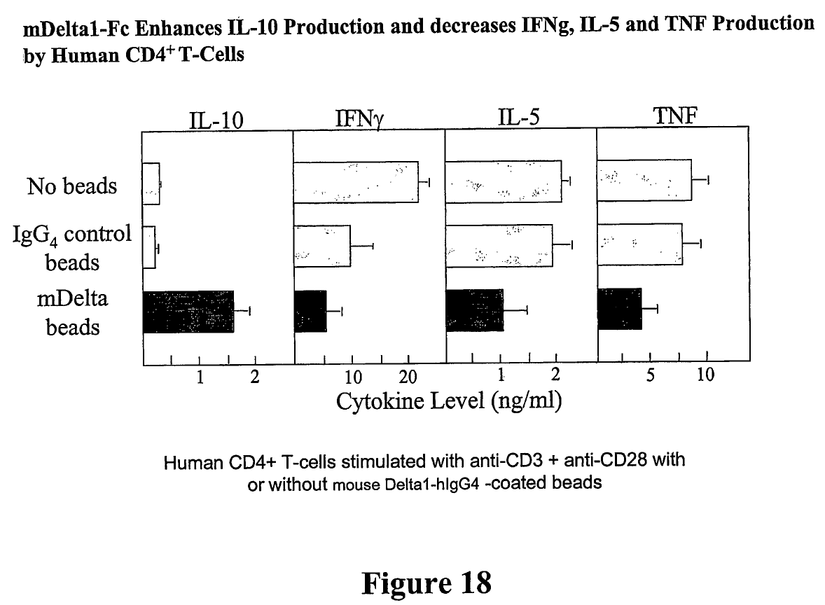

Results are shown in FIGS. 16 to 18.

### Example 12

### Variation of Bead:Cell Ratios

The procedure of Example 11 was repeated except that the ratio of control beads to cells and mouse Delta1-hIgG4 fusion protein coated beads to cells was varied between 16:1 and 0.25:1 (variously 16:1, 8:1, 4:1, 2:1, 1:1, 0.5:1, 0.25:1) and human Delta1-hIgG4 fusion protein coated beads were also used at the same ratios for comparison.

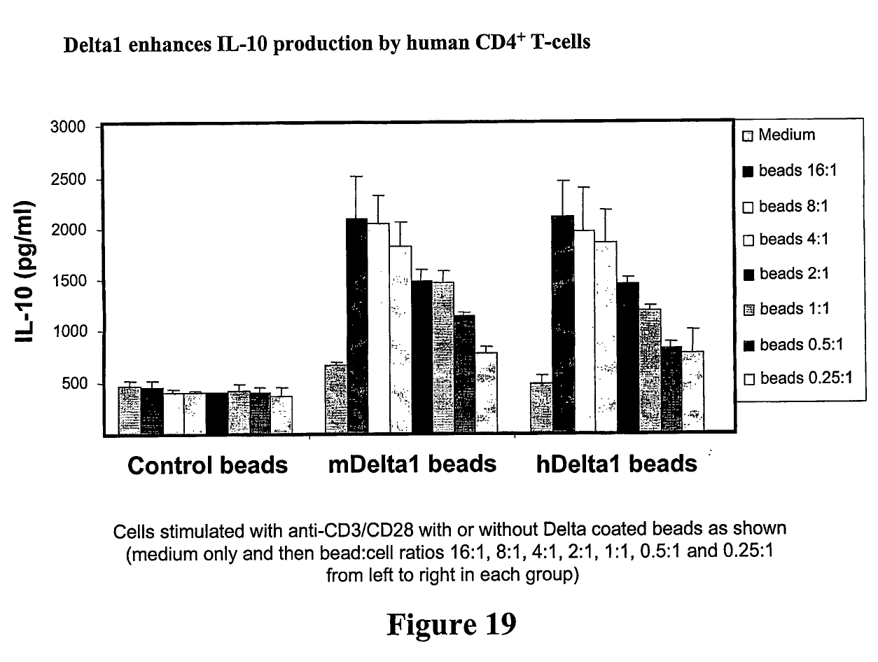

Results are shown in FIG. 19.

### Example 13

### Comparison of CD45RO+ (Memory Cells) and CD45RO− (Naive Cells)

The procedure of Example 11 was repeated except that prior to the stimulation the human CD4+ were separated into CD45RO+ (memory cells) and CD45RO− (naive cells, data not shown on the slide). The magnetic separation was done using anti-CD4 Multisort microbeads (cat.No. 551-01) and then anti-CD45RO microbeads (cat.No. 460-01) supplied by Miltenyi Biotech and following Miltenyi's protocol.

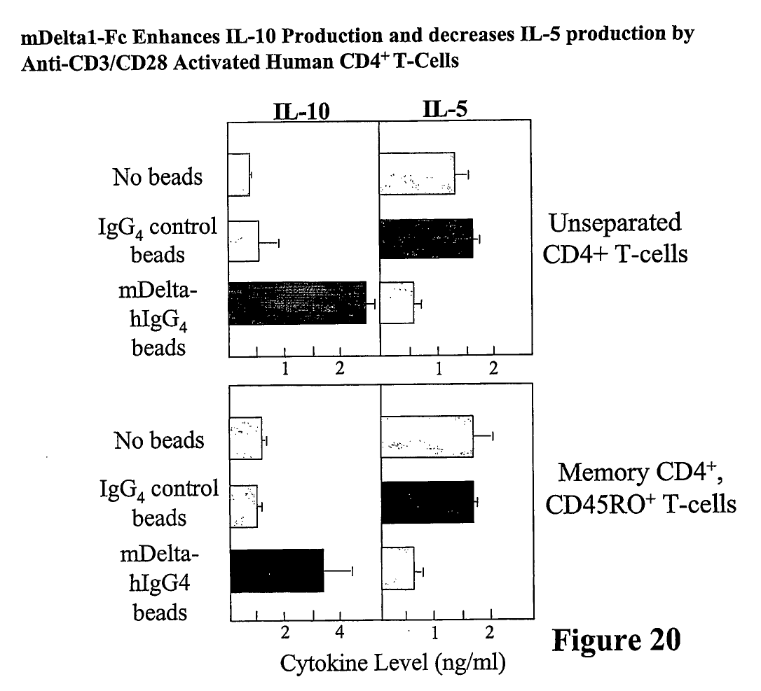

Results are shown in FIG. 20.

### Example 14

### Measurement of Cytokine Production in Stimulated Mouse CD4+ Cells Under Polarising Conditions

(i) CD4+ Cell Purification

Spleens were removed from mice (variously Balb/c females, 8-10 weeks, C57B/6 females, 8-10 weeks, CARD 1 females, 8-10 weeks (D011.10 transgenic, CAR transgenic)) and passed through a 0.2 μM cell strainer into 20 ml R10F medium (R10F-RPMI 1640 media (Gibco Cat No 22409) plus 2 mM L-glutamine, 50 μg/ml Penicillin, 50 μg/ml Streptomycin, 5×10⁻⁵ M β-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

(ii) Antibody Coating

96 well flat-bottomed plates were coated with Dulbecco's Phosphate Buffered Saline (DPBS) plus 1 μg/ml anti-CD3 antibody (Pharmingen, San Diego, US: Cat No 553058, Clone No 145-2C11) plus 1 μg/ml anti-IgG4 antibody (Pharmingen Cat No 555878). 100 μl of coating mixture was used per well. Plates were incubated overnight at 4° C. then washed with DPBS. Each well then received either 100 μl DPBS or 100 μl DPBS plus 10 μg/ml Notch ligand (mouse Delta 1 extracellular domain/Ig4Fc fusion protein; Fc-delta). The plates were incubated for 2-3 hours at 37° C. then washed again with DPBS before cells (prepared as in (i)) were added.

(iii) Primary Polyclonal Stimulation

CD4+ cells were cultured in 96 well, flat-bottomed plates pre-coated as in (ii) above. Cells were re-suspended, following counting, at 2×10⁶/ml in R10F medium plus 4 μg/ml anti-CD28 antibody (Pharmingen, Cat No 553294, Clone No 37.51). 100 μl cell suspension was added per well. 100 μl of polarising or control medium was then added to each well to give a final volume of 200 μl (2×10⁵ cells/well, anti-CD28 final concentration 2 μg/ml) as follows:

Un-polarised cells: R10F medium.

Th1 polarised cells: R10F medium plus anti-IL-4 antibody (10 μg/ml, Pharmingen Cat No 554432) plus IL-12 (10 ng/ml, Peprotech 210-12).

Th2 polarised cells: R10Fmedium plus anti-IL-12 antibody (10 μg/ml, Pharmingen Cat No 554475) plus anti-IFNg antibody (1 μg/ml, Pharmingen Cat No 554408) plus IL-4 (10 ng/ml, Peprotech Cat No 214-14).

The plates were then incubated at 37° C. for 72 hours.

125 μl supernatant was then removed from each well and stored at −20° C. until tested by ELISA for IL-10 and TNFa using antibody pairs from R & D Systems (Abingdon, UK). The cells were then split 1 in 3 into new wells (not coated) and fed with R10F medium plus recombinant human IL-2 (2.5 ng/ml, PeproTech Inc, London, UK: Cat No 200-02).

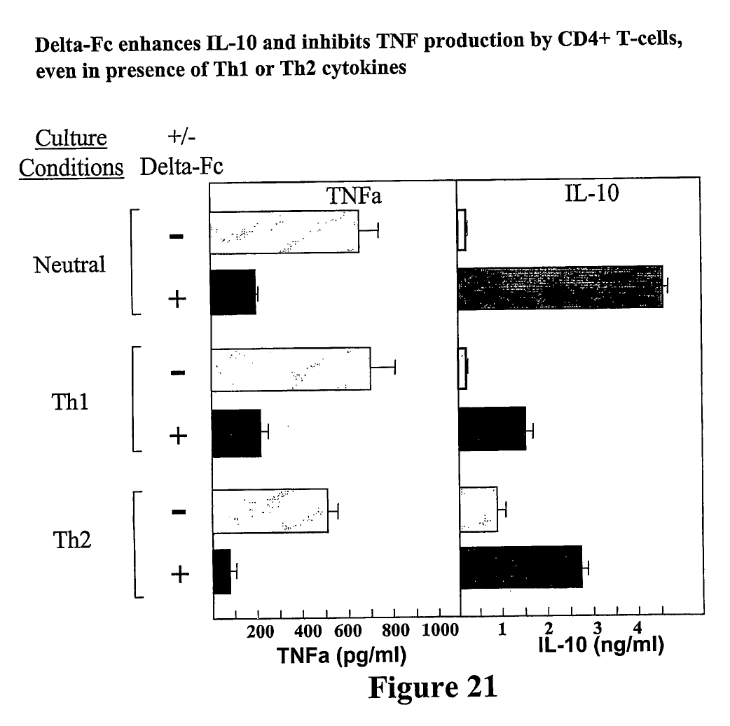

Results are shown in FIG. 21.

### Example 15

### Preparation of Human Delta1-IgG4Fc Fusion Protein

A fusion protein comprising the extracellular domain of human Delta1 fused to the Fc domain of human IgG4 (referred to herein as “hDelta1-IgG4Fc” and also referred to in the accompanying Figures as “D1E8G4” and “D1E8Fc4”) was prepared by inserting a nucleotide sequence coding for the extracellular domain of human Delta1 (see e.g., Genbank Accession No AF003522) into the expression vector pCONγ (Lonza Biologics, Slough, UK) and expressing the resulting construct in CHO cells.

i) Cloning

A 1622 bp extracellular (EC) fragment of human Delta-like ligand 1 (hECDLL-1; see GenBank Accession No AF003522) was gel purified using a Qiagen QIAquick™ Gel Extraction Kit (cat 28706) according to the manufacturer's instructions (Qiagen, Valencia, Calif., US). The fragment was then ligated into a pCR Blunt cloning vector (Invitrogen, UK) cut HindIII-BsiWI, thus eliminating a HindIII, BsiWI and ApaI site.

The ligation was transformed into DH5α cells, streaked onto LB+Kanamycin (30 ug/ml) plates and incubated at 37° C. overnight. Colonies were picked from the plates into 3 ml LB+Kanamycin (30 ugml⁻¹) and grown up overnight at 37° C. Plasmid DNA was purified from the cultures using a Qiagen Qiaquick Spin Miniprep kit (cat 27106) according to the manufacturer's instructions, then diagnostically digested with HindIII. A clone was chosen and streaked onto an LB+Kanamycin (30 ug/ml) plate with the glycerol stock of modified pCRBlunt-hECDLL-1 and incubated at 37° C. overnight. A colony was picked off this plate into 60 ml LB+Kanamycin (30 ug/ml) and incubated at 37° C. overnight. The culture was maxiprepped using a Clontech Nucleobond Maxi Kit (cat K3003-2) according to the manufacturer's instructions (Clontech, PaloAlto, Calif., US), and the final DNA pellet was resuspended in 300 ul dH₂O and stored at −20° C.

5 ug of modified pCR Blunt-hECDLL-1 vector was linearised with HindIII and partially digested with ApaI. The 1622 bp HECDLL-1 fragment was then gel purified using a Clontech Nucleospin® Extraction Kit (K3051-1) according to the manufacturer's instructions. The DNA was then passed through another Clontech Nucleospin® column and followed the isolation from PCR protocol, concentration of sample was then checked by agarose gel analysis ready for ligation.

Plasmid pconγ (Lonza Biologics, UK) was cut with HindIII-ApaI and the following oligos (SEQ ID NOs:35 and 36, respectively) were ligated in:

```
agcttgcggc cgcgggccca gcggtggtgg acctcactga
gaagctagag gcttccacca aaggcc
        acgccg gcgcccgggt cgccaccacc tggagtgact
        cttcgatctc cgaaggtggt tt
```

The ligation was transformed into DH5α cells and LB+Amp (100 ug/ml) plates were streaked with 200 ul of the transformation and incubated at 37° C. overnight. The following day 12 clones were picked into 2×YT+Ampicillin (100 ugml⁻¹) and grown up at 37° C. throughout the day. Plasmid DNA was purified from the cultures using a Qiagen Qiaquick Spin Miniprep kit (cat 27106) and diagnostically digested with NotI. A clone (designated “pDev41”) was chosen and an LB+Amp (100 ug/ml) plate was streaked with the glycerol stock of pDev41 and incubated at 37° C. overnight. The following day a clone was picked from this plate into 60 ml LB+Amp (100 ug/ml) and incubated with shaking at 37° C. overnight. The clone was maxiprepped using a Clontech Nucleobond Maxi Kit (cat K3003-2) according to the manufacturer's instructions and stored at −20° C.

The pDev41 clone 5 maxiprep was then digested with ApaI-EcoRI to generate the IgG4Fc fragment (1624 bp). The digest was purified on a 1% agarose gel and the main band was cut out and purified using a Clontech Nucleospin Extraction Kit (K3051-1).

The polynucleotide was then cloned into the polylinker region of pEE14.4 (Lonza Biologics, UK) downstream of the strong hCMV promoter enhancer region (hCMV-MIE) and upstream of SV40 polyadenylation signal (encodes the GS gene required for selection in glutamine free media; contains the GS minigene—GS cDNA which includes the last intron and polylinker adenylation signals of the wild type hamster GS gene) which is under the control of the late SV40 promoter, has the hCMV promoter to drive transcription of the desired gene. 5 ug of the maxiprep of pEE14.4 was digested with HindIII-EcoRI, and the product was gel extracted and treated with alkaline phosphatase.

ii) Generation of Expression Constructs

A 3 fragment ligation was set up with pEE14.4 cut HindIII-EcoRI, ECDLL-1 from modified pCR Blunt (HindIII-ApaI) and the IgG4Fc fragment cut from pDev41 (ApaI-EcoRI). This was transformed into DH5α cells and LB+Amp (100 ug/ml) plates were streaked with 200 ul of the transformation and incubated at 37 C overnight. The following day 12 clones were picked into 2×YT+Amp (100 ug/ml) and minipreps were grown up at 37° C. throughout the day. Plasmid DNA was purified from the preps using a Qiagen Qiaquick spin miniprep kit (Cat No 27106), diagnostically digested (with EcoRI and HindIII) and a clone (clone 8; designated “pDev44”) was chosen for maxiprepping. The glycerol stock of pDev44 clone 8 was streaked onto an LB+Amp (100 ugml⁻¹) plate and incubated at 37° C. overnight. The following day a colony was picked into 60 ml LB+Amp (100 ugml⁻) broth and incubated at 37° C. overnight. The plasmid DNA was isolated using a Clontech Nucleobond Maxiprep Kit (Cat K3003-2).

iii) Addition of Optimal KOZAK Sequence

A Kozak sequence was inserted into the expression construct as follows. Oligonucleotides were kinase treated and annealed to generate the following sequences:

(SEQ ID NO: 37)

AGCTTGCCGCCACCATGGGCAGTCGGTGCGCGCTGGCCCTGGCGGTGCTC

(SEQ ID NO: 38)

ACGGCGGTGGTACCCGTCAGCCACGCGCGACCGGGACCGC

(SEQ ID NO: 39)

TCGGCCTTGCTGTGTCAGGTCTGGAGCTCTGGGGTGTT

(SEQ ID NO: 40)

CACGAGAGCCGGAACGACACAGTCCAGACCTCGAGACCCCACAAGC

pDev44 was digested with HindIII-BstBI, gel purified and treated with alkaline phosphatase. The digest was ligated with the oligos, transformed into DH5α cells by heat shock. 200 ul of each transformation were streaked onto LB+Amp plates (10 ug/ml) and incubated at 37° C. overnight. Minipreps were grown up in 3 ml 2×YT+Ampicillin (100 ugml⁻¹). Plasmid DNA was purified from the minipreps using a Qiagen Qiaquick spin miniprep kit (Cat No 27106) and diagnostically digested with NcoI. A clone (pDev46) was selected and the sequence was confirmed. The glycerol stock was streaked, broth grown up and the plasmid maxiprepped.
iv) Transfection

Approx 100 ug pDev46 Clone 1 DNA was linearised with restriction enzyme Pvu I. The resulting DNA preparation was cleaned up using phenol/chloroform/IAA extraction followed by ethanol wash and precipitation. The pellets were resuspended in sterile water and linearisation and quantification was checked by agarose gel electrophoresis and UV spectrophotometry.

40 ug linearised DNA (pDev46 Clone 1) and 1×10⁷ CHO-K1 cells were mixed in serum free DMEM in a 4 mm cuvette, at room temp. The cells were then electroporated at 975 uF 280 volts, washed out into non-selective DMEM, diluted into 96 well plates and incubated. After 24 hours media were removed and replaced with selective media (25 uM L-MSX). After 6 weeks media were removed and analysed by IgG4 sandwich ELISA.

Selective media were replaced. Positive clones were identified and passaged in selective media 25 um L-MSX.

v) Expression

Cells were grown in selective DMEM (25 um L-MSX) until semi-confluent. The media was then replaced with serum free media (UltraCHO) for 3-5 days. Protein (hDelta1-IgG4Fc fusion protein) was purified from the resulting media by FPLC.

The amino acid sequence of the resulting expressed fusion protein was as follows (SEQ ID NO:41):

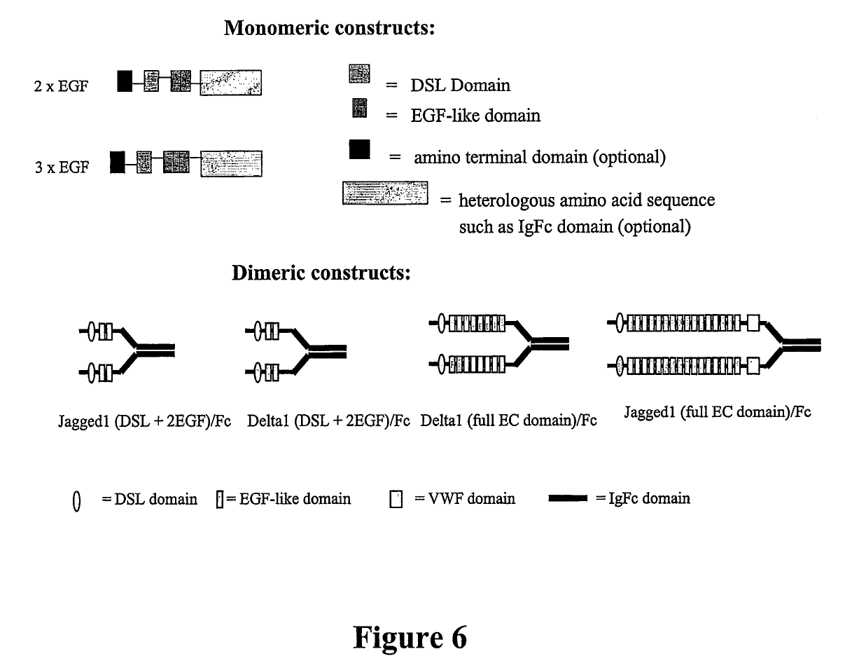

MGSRCALALAVLSALLCQVWSSGVFELKLQEFVNKKGLLGNRNCCRGGAGPPPCA
CRTFFRVCLKHYQASVSPEPPCTYGSAVTPVLGVDSFSLPDGGGADSAFSNPIRFPFG
FTWPGTFSLIIEALHTDSPDDLATENPERLISRLATQRHLTVGEEWSQDLHSSGRTDL
KYSYRFVCDEHYYGEGCSVFCRPRDDAFGHFTCGERGEKVCNPGWKGPYCTEPICL
PGCDEQHGFCDKPGECKCRVGWQGRYCDECIRYPGCLHGTCQQPWQCNCQEGWG
GLFCNQDLNYCTHHKPCKNGATCTNTGQGSYTCSCRPGYTGATCELGIDECDPSPC
KNGGSCTDLENSYSCTCPPGFYGKICELSAMTCADGPCFNGGRCSDSPDGGYSCRCP
VGYSGFNCEKKIDYCSSSPCSNGAKCVDLGDAYLCRCQAGFSGRHCDDNVDDCASS
PCANGGTCRDGVNDFSCTCPPGYTGRNCSAPVSRCEHAPCHNGATCHERGHGYVCE
CARGYGGPNCQFLLPELPPGPAVVDLTEKLEASTKGPSVFPLAPCSRSTSESTAALGC
LVKDYFPEPVTVSWNSGALTSGVHTFPAVLQSSGLYSLSSVVTVPSSSLGTKTYTCN
VDHKPSNTKVDKRVESKYGPPCPSCPAPEFLGGPSVFLFPPKPKDTLMISRTPEVTCV
VVDVSQEDPEVQFNWYVDGVEVHNAKTKPREEQFNSTYRVVSVLTVLHQDWLNG
KEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMH
EALHNHYTQKSLSLSLGK

Wherein the first underlined sequence is the predicted signal peptide (cleaved from the mature protein) and the second underlined sequence is the IgG4 Fc sequence. The protein normally exists as a dimer linked by cysteine disulphide bonds (see e.g. schematic representation in FIG. 6).

### Example 16

### Preparation of Notch Ligand Extracellular Comain Fragment with Free Cysteine Tail for Particle Coupling

A protein fragment comprising amino acids 1 to 332 of human Delta 1 (DLL-1; for sequence see GenBank Accession No AF003522) and ending with a free cysteine residue (“D1E3cys”) was prepared as follows:

A template containing the entire coding sequence for the extracellular (EC) domain of human DLL-1 (with two silent mutations) was prepared by a PCR cloning strategy from a placental cDNA library made from placental polyA+ RNA (Clontech; cat no 6518-1) and combined with a C-terminal V5HIS tag in a pCDNA3.1 plasmid (Invitrogen, UK) The template was cut HindIII to PmeI to provide a fragment coding for the EC domain and this was used as a template for PCR using primers as follows:

| | | |
|---|---|---|
| 5′- | CAC CAT GGG CAG TCG GTG | (SEQ ID NO: 42) |
| primer: | CGC GCT GG | |
| 3′- | GTC TAC GTT TAA ACT TAA | (SEQ ID NO: 43) |
| primer: | CAC TCG TCA ATC CCC AGC | |
| | TCG CAG GTG | |

PCR was carried out using Pfu turbo polymerase (Stratagene, La Jolla, Calif., US) with cycling conditions as follows: 95 C 5 min, 95 C 1 min, 45-69 C 1 min, 72 C 1 min for 25 cycles, 72 C 10 min.

The products at 58 C, 62 C & 67 C were purified from 1% agarose gel in 1×TAE using a Qiagen gel extraction kit according to the manufacturer's instructions, ligated into pCRIIblunt vector (InVitrogen TOPO-blunt kit) and then transformed into TOP10 cells (InVitrogen). The resulting clone sequence was verified, and only the original two silent mutations were found to be present in the parental clone.

The resulting sequence coding for “D1E3Cys” was excised using PmeI and HindIII, purified on 1% agarose gel, 1×TAE using a Qiagen gel extraction kit and ligated into pCDNA3.1V5HIS (Invitrogen) between the PmeI and HindIII sites, thereby eliminating the V5HIS sequence. The resulting DNA was transformed into TOP10 cells. The resulting clone sequence was verified at the 3′-ligation site.

The D1E3Cys-coding fragment was excised from the pCDNA3.1 plasmid using PmeI and HindIII. A pEE14.4 vector plasmid (Lonza Biologics, UK) was then restricted using EcoRI, and the 5′-overhangs were filled in using Klenow fragment polymerase. The vector DNA was cleaned on a Qiagen PCR purification column, restricted using HindIII, then treated with Shrimp Alkaline Phosphatase (Roche). The pEE14.4 vector and D1E3cys fragments were purified on 1% agarose gel in 1×TAE using a Qiagen gel extraction kit prior to ligation (T4 ligase) to give plasmid pEE14.4 DLLΔ4-8cys. The resulting clone sequence was verified.

The D1E3Cys coding sequence is as follows (SEQ ID NO:44):

| | |
|---|---|
| 1 | atgggcagtc ggtgcgcgct ggccctggcg gtgctctcgg |
| | ccttgctgtg |
| 51 | tcaggtctgg agctctgggg tgttcgaact gaagctgcag |
| | gagttcgtca |
| 101 | acaagaaggg gctgctgggg aaccgcaact gctgccgcgg |
| | gggcgcgggg |
| 151 | ccaccgccgt gcgcctgccg gaccttcttc cgcgtgtgcc |
| | tcaagcacta |
| 201 | ccaggccagc gtgtcccccg agccgccctg cacctacggc |
| | agcgccgtca |
| 251 | cccccgtgct gggcgtcgac tccttcagtc tgcccgacgg |
| | cgggggcgcc |
| 301 | gactccgcgt tcagcaaccc catccgcttc cccttcggct |
| | tcacctggcc |
| 351 | gggcaccttc tctctgatta ttgaagctct ccacacagat |
| | tctcctgatg |
| 401 | acctcgcaac agaaaaccca gaaagactca tcagccgcct |
| | ggccacccag |
| 451 | aggcacctga cggtgggcga ggagtggtcc caggacctgc |
| | acagcagcgg |
| 501 | ccgcacggac ctcaagtact cctaccgctt cgtgtgtgac |
| | gaacactact |
| 551 | acggagaggg ctgctccgtt ttctgccgtc cccgggacga |
| | tgccttcggc |
| 601 | cacttcacct gtggggagcg tggggagaaa gtgtgcaacc |
| | ctggctggaa |
| 651 | agggccctac tgcacagagc cgatctgcct gcctggatgt |
| | gatgagcagc |
| 701 | atggattttg tgacaaacca ggggaatgca agtgcagagt |
| | gggctggcag |
| 751 | ggccggtact gtgacgagtg tatccgctat ccaggctgtc |
| | tccatggcac |
| 801 | ctgccagcag ccctggcagt gcaactgcca ggaaggctgg |
| | gggggccttt |
| 851 | tctgcaacca ggacctgaac tactgcacac accataagcc |
| | ctgcaagaat |
| 901 | ggagccacct gcaccaacac gggccagggg agctacactt |
| | gctcttgccg |
| 951 | gcctgggtac acaggtgcca cctgcgagct ggggattgac |
| | gagtgttaa |

The DNA was prepared for stable cell line transfection/selection in a Lonza GS system using a Qiagen endofree maxi-prep kit.

Expression of D1E3Cys

Linearisation of DNA

The pEE14.4 DLLΔ4-8cys plasmid DNA from (i) above was linearised by restriction enzyme digestion with PvuI, and then cleaned up using phenol chloroform isoamyl alcohol (IAA), followed by ethanol precipitation. Plasmid DNA was checked on an agarose gel for linearisation, and spec'd at 260/280 nm for quantity and quality of prep.

Transfection

CHO-K1 cells were seeded into 6 wells at 7.5×10⁵ cells per well in 3 ml media (DMEM 10% FCS) 24 hrs prior to transfection, giving 95% confluency on the day of transfection.

Lipofectamine 2000 was used to transfect the cells using 5 ug of linearised DNA. The transfection mix was left on the cell sheet for 5½ hours before replacing with 3 ml semi-selective media (DMEM, 10% dFCS, GS) for overnight incubation.

At 24 hours post-transfection the media was changed to full selective media (DMEM (Dulbecco's Modified Eagle Medium), 10% dFCS (fetal calf serum), GS (glutamine synthase), 25 uM L-MSX (methionine sulphoximine)) and incubated further.

Cells were plated into 96 wells at 10⁵ cells per well on days 4 and 15 after transfection.

96 well plates were screened under a microscope for growth 2 weeks post clonal plating. Single colonies were identified and scored for % confluency. When colony size was >30% media was removed and screened for expression by dot blot against anti-human-Delta-1 antisera. High positives were confirmed by the presence of a 36 kDa band reactive to anti-human-Delta-1 antisera in PAGE Western blot of media.

Cells were expanded by passaging from 96 well to 6 well to T25 flask before freezing. The fastest growing positive clone (LC09 0001) was expanded for protein expression.

D1E3Cys Expression and Purification

T500 flasks were seeded with 1×10⁷ cells in 80 ml of selective media. After 4 days incubation the media was removed, cell sheet rinsed with DPBS and 150 ml of 325 media with GS supplement added to each flask. Flasks were incubated for 7 further days before harvesting. Harvest media was filtered through a 0.65-0.45 um filter to clarify prior to freezing. Frozen harvests were purified by FPLC as follows:

Frozen harvest was thawed and filtered. A 17 ml Q Sepharose column was equilibrated in 0.1M Tris pH8 buffer, for 10 column volumes. The harvest was loaded onto the column using a P1 pump set at 3 ml/min, the flowthrough was collected into a separate container (this is a reverse purification—a lot of the BSA contaminant binds to the Q Sepharose FF and our target protein does not and hence remains in the flowthrough). The flowthrough was concentrated in a TFF rig using a 10 kDa cut off filter cartridge, during concentration it was washed 3× with 0.1M Sodium phosphate pH 7 buffer. The 500 ml was concentrated down to 35 ml, to a final concentration of 3 mg/ml.

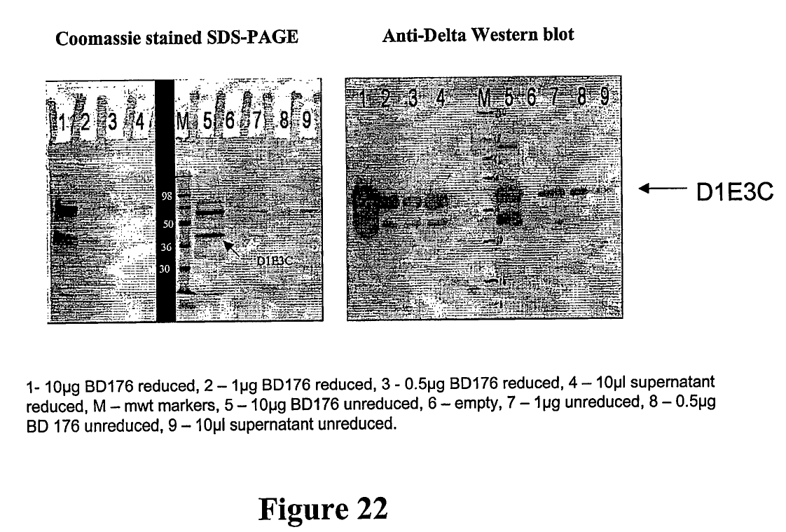

Samples were run on SDS PAGE reduced and non-reduced (gels are shown in FIG. 22).

The amino acid sequence of the resulting expressed D1E3Cys protein was as follows (SEQ ID NO:1):

MGSRCALALAVLSALLCQVWSSGVFELKLQEFVNKKGLLGNRNCCRGGAGPPPCACRTF
FRVCLKHYQASVSPEPPCTYGSAVTPVLGVDSFSLPDGGGADSAFSNPIRFPFGFTWPG
TFSLIIEALHTDSPDDLATENPERLISRLATQRHLTVGEEWSQDLHSSGRTDLKYSYRF
VCDEHYYGEGCSVFCRPRDDAFGHFTCGERGEKVCNPGWKGPYCTEPICLPGCDEQHGF
CDKPGECKCRVGWQGRYCDECIRYPGCLHGTCQQPWQCNCQEGWGGLFCNQDLNYCTHH
KPCKNGATCTNTGQGSYTCSCRPGYTGATCELGIDEC

(wherein the sequence in italics is the leader peptide, the underlined sequence is the DSL domain, the bold sequences are the three EGF repeats, and the terminal Cys residue is shown bold underlined).
Reduction of D1E3cys Protein

40 μg D1E3Cys protein from above was made up to 100 μl to include: 100 mM sodium phosphate pH 7.0 and 5 mM EDTA. 2 volumes of immobilised TCEP (tris[2-carboxyethyl]phosphine hydrochloride; Pierce, Rockford, Ill., US, Cat No: 77712; previously washed 3 times 1 ml 100 mM sodium phosphate pH 7.0) were added and the mixture was incubated for 30 minutes at room temperature, with rotating.

The resin was pelleted at room temperature in a microfuge (13,000 revs/min, 5 minutes) and the supernatant was transferred to a clean Eppendorf tube and stored on ice. Protein concentration was measured by Warburg-Christian method.

### Example 17

### Coating M-450 Epoxy Dynabeads with Notch Ligand Proteins

Dynabeads M-450 Epoxy (Dynal, Cat. no. 140.01; 4.5 μm average diameter) are supplied by Dynal (Dynal Biotech, Oslo, Norway) as ethanol-washed beads in distilled water at 4×10⁸ beads/ml. These beads are magnetic polystyrene beads that have a surface epoxy (glycidyl ether) reactive group which does not require further activation.

Proteins are adsorbed hydrophobically on initial coupling with covalent coupling of primary amine groups occurring after 24 h. Coupling reactions occur at neutral pH over a 24 h incubation time at a temperature between 4° C. and 37° C.

The appropriate quantity of epoxy beads were washed in PBS using a magnet (3×1 ml).

Purified hDelta1-IgG4Fc fusion protein from Example 15 or D1E3Cys protein from Example 16 above was added (˜5 μg per 10⁷ beads typical starting concentration) to beads at a final concentration of 4-8×10⁸ beads/ml.

In some cases a blocking protein was added to assist binding orientation as follows: The beads were incubated for 15-30 min at 4-37° C., with shaking. 0.1% BSA was added as blocking protein (Dynal typically suggest 0.1-0.5% BSA or HAS for antibody binding) The beads were then left for 16-20 h at 4-37° C., depending on stability of protein, with shaking to ensure covalent coupling. Coated beads were washed×3 with PBS/0.1% BSA using a magnet. The addition of 0.1% BSA in the wash buffer here ensures complete blocking of the beads after coating. Coated beads were stored at 4° C.

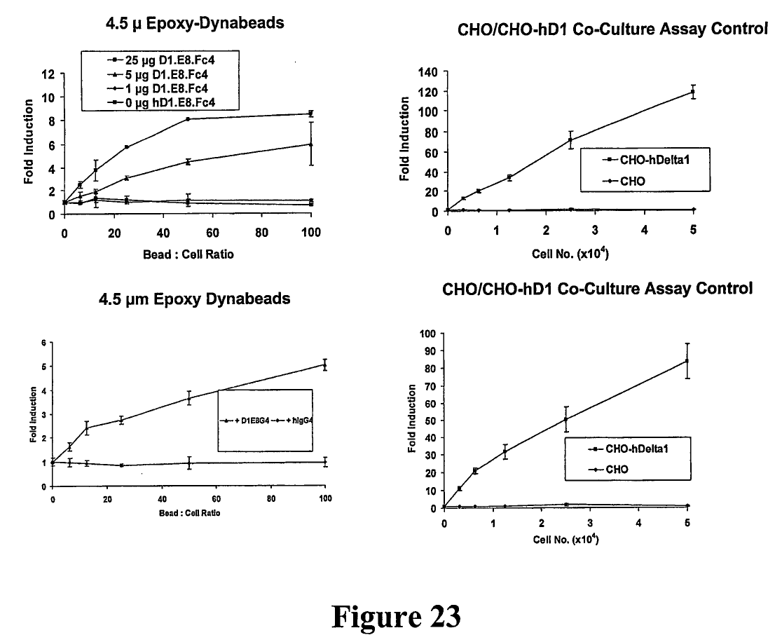

The activity of the various beads was tested in a CHO-N2 stable reporter assay as described above (with parallel experiments using hIgG4-coated beads under the same conditions as control). CHO/CHO-hD1 co-culture assays (using CHO cells expressing full length Delta 1 or native CHO cells in place of beads) were run at the same time as further controls. Results are shown in FIG. 23.

### Example 18

### Coating M-270 Amine Dynabeads with Notch Ligand Protein

To activate, 1.5×10⁸ M-270 Amine Dynabeads (2.7 μm average diameter) were washed 3 times with 0.5 ml 100 mM sodium phosphate pH 7.0, then made to 2.5×10⁸/ml in 100 mM sodium phosphate pH 7.

Sulfo-SMCC (sulphosuccinimidyl 4-(N-maleimidomethyl)cyclohexane-1-carboxylate; Pierce, Cat No: 22322) was added to 0.2 mg/ml and the mixture was incubated for 30 minutes at room temperature, with rotating. The beads were washed 3 times with 0.5 ml 100 mM sodium phosphate pH 7.0, then made up to 1×10⁹/ml in 100 mM sodium phosphate pH 7.

5×10⁷ ‘activated’ amine beads in 100 mM sodium phosphate pH7 were combined with 20 ug TCEP reduced D1E3cys protein from Example 16 above and made up to 250 μl in sodium phosphate pH 7. The resulting mixture was incubated for 18 hours at 4 C with rotation.

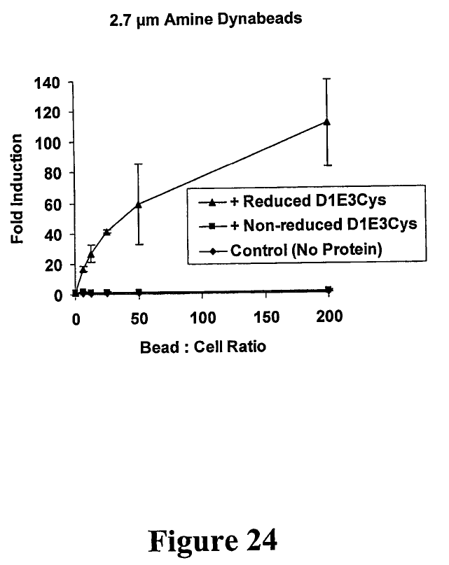

The resulting protein coupled beads were washed 3 times with 0.5 ml DPBS, then made up to 2×10⁸/ml in DPBS. Activity was measured using a CHO-Notch 2 reporter assay as described above (with parallel experiments using non-reduced D1E3Cys protein-coated beads and uncoated beads under the same conditions as controls). Results are shown in FIG. 24.

### Example 19

### Coating Sulphate-Polystyrene/Latex Beads with Notch Ligand Protein

Surfactant-free sulphate white polystyrene latex beads (product number 1-5000) were supplied by Interfacial Dynamics Corporation (Portland, Oreg., US). The polystyrene microspheres have a mean diameter of 4.9 μm and were supplied dispersed in distilled de-ionised water at 6.3×10⁸ beads/ml. The beads are negatively charged and have sulphate groups on the surface—the surface is hydrophobic in nature.

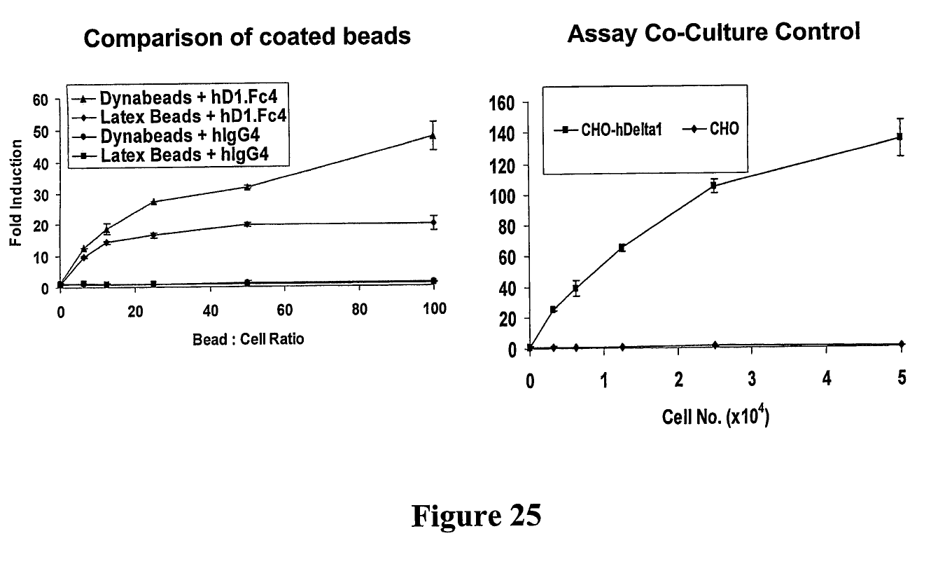

To coat the beads with D1E8G4 (prepared as above) or human IgG4 (hIgG4, Sigma; as a control) an aliquot of the supplied beads was removed and placed into 1 ml of sterile PBS, spun at 13K for 10 min and re-washed with a further 1 ml of PBS. The beads were resuspended in PBS at 50 μl per 10⁷ beads. Protein used to coat the beads was added at a concentration of 10 μg per 10⁷ beads in a final concentration of 200 μg/ml in PBS in a 500 μl Eppendorf tube and placed on a rotating wheel overnight at 4° C. The following day the beads were washed by pelleting in a microfuge at 13 K for 10 min and washing with 3×1 ml of PBS. After the final wash the beads were resuspended in complete medium (DMEM+10% HI FCS+glutamine+P/S) at 2×10⁷ beads/ml and assayed in the CHO-N2 signalling assay starting at 2×10⁶ beads per well with serial 1:2 dilutions of the beads made in complete medium. Results are shown in FIG. 25.

### Example 20

### CD4+ Cell Purification

Spleens were removed from mice (Balb/c females, 8-10 weeks) and treated with 1 mg/ml Collagenase D (Boehringer Mannheim) in RPMI medium with no supplements for 40 min. Tissue was passed through a 0.2μ cell strainer (Falcon) into 20 ml R10F medium (R10F—RPMI 1640 media (Gibco Cat No 22409) plus 2 mM L-glutamine, 50 μg/ml Penicillin, 50 μg/ml Streptomycin, 5×10⁻⁵ Mβ-mercapto-ethanol in 10% fetal calf serum). The cell suspension was spun (1150 rpm 5 min) and the media removed.

The cells were incubated for 4 minutes with 5 ml ACK lysis buffer (0.15M NH₄Cl, 1.0M KHCO₃, 0.1 mM Na₂EDTA in double distilled water) per spleen (to lyse red blood cells). The cells were then washed once with R10F medium and counted. CD4+ cells were purified from the suspensions by positive selection on a Magnetic Associated Cell Sorter (MACS) column (Miltenyi Biotec, Bisley, UK: Cat No 130-042-401) using CD4 (L3T4) beads (Miltenyi Biotec Cat No 130-049-201), according to the manufacturer's directions.

### Example 21

### Antibody Coating

The following protocol was used for coating 96 well flat-bottomed plates with antibodies.

The plates were coated with Dulbecco's Phosphate Buffered Saline (DPBS) plus 1 μg/mL anti-hamster IgG antibody (Pharmingen, San Diego, US: Cat No 554007). 100 μL of coating mixture was used per well. Plates were incubated overnight at 4° C. then washed with DPBS. Each well then received 100 μL DPBS plus 0.1-1 μg/mL anti-CD3 (Pharmingen Cat No 553058, Clone No 145-2C11).

The plates were incubated for 2-3 hours at 37° C. then washed again with DPBS before cells (prepared as in Example 20) were added.

### Example 22

### Primary Polyclonal Stimulation

CD4+ cells were cultured in 96 well, flat-bottomed plates pre-coated according to Example 21. Cells were resuspended following counting at 4×10⁶/mL in R10F medium and 50 μL suspension added per well. R10F medium plus 8 μg/mL CD28 antibody (Pharmingen, Cat No 553294, Clone No 37.51) was added at 50 μL per well. Beads coated with Notch ligand were added in appropriate volumes to give final ratios of 0.1-20:1 beads:cell. R10F medium was added to give a final volume of 200 μL per well (2×10⁵ cells/well, anti-CD28 final concentration 2 μg/mL). The plates were then incubated at 37° C. for 72 hours.

170 μL supernatant was then removed from each well and stored at −20° C. until tested by ELISA for IL-10, IFNγ, IL-2 and IL-13 using antibody pairs from R&D Systems (Abingdon, UK).

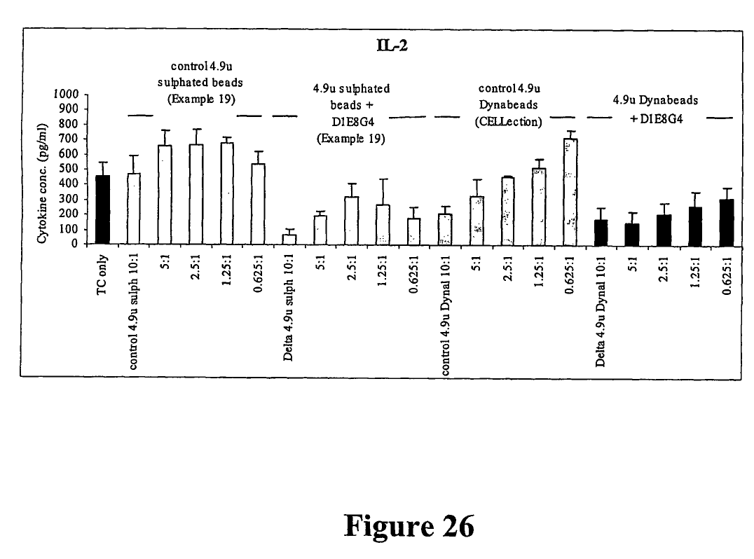

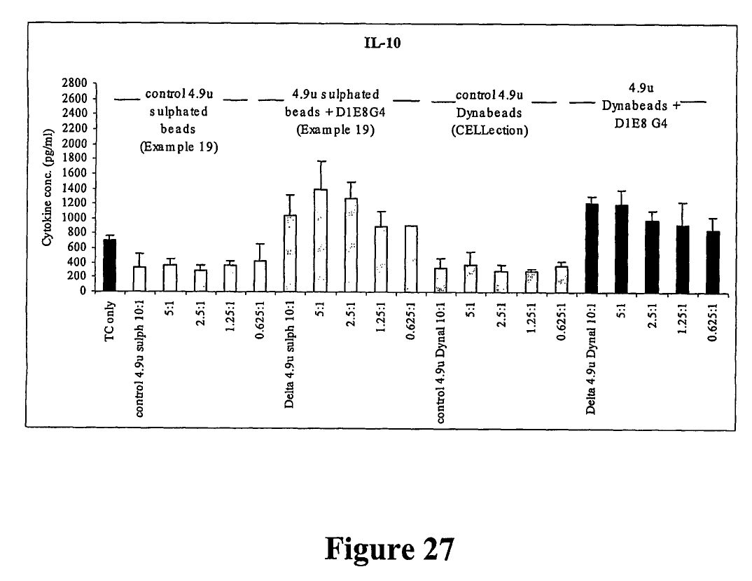

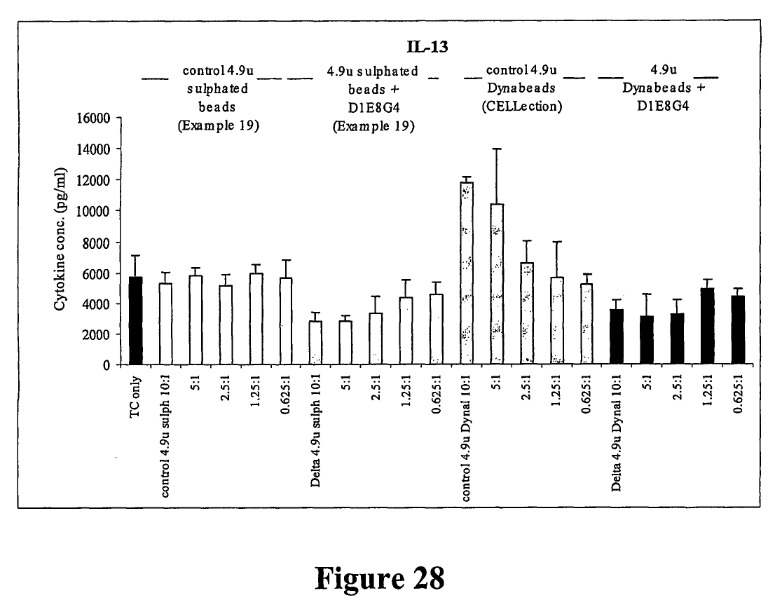

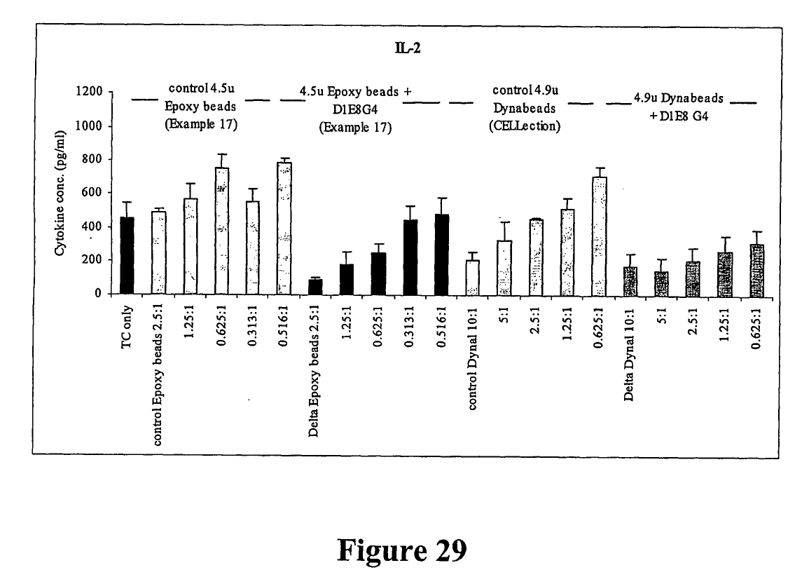

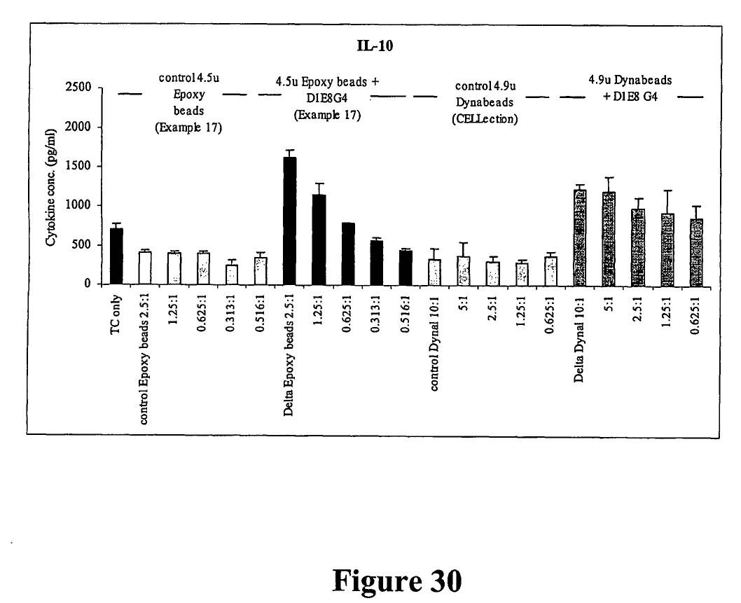

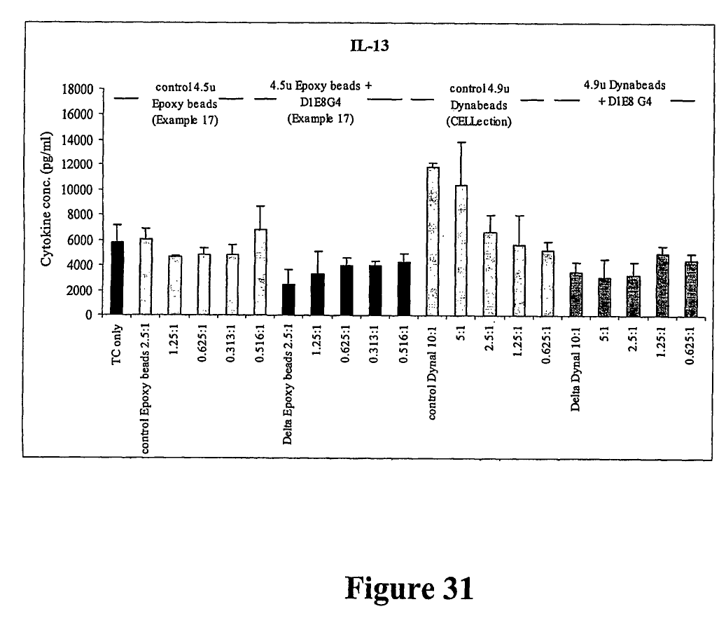

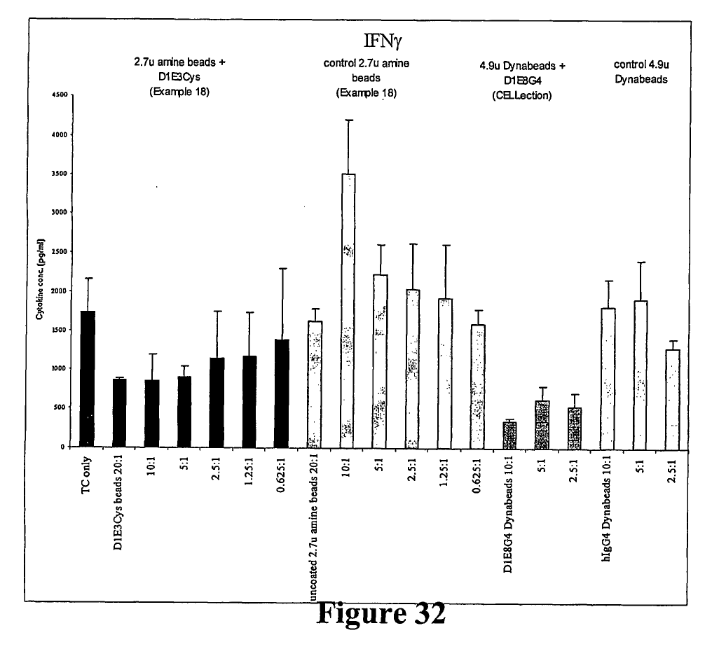

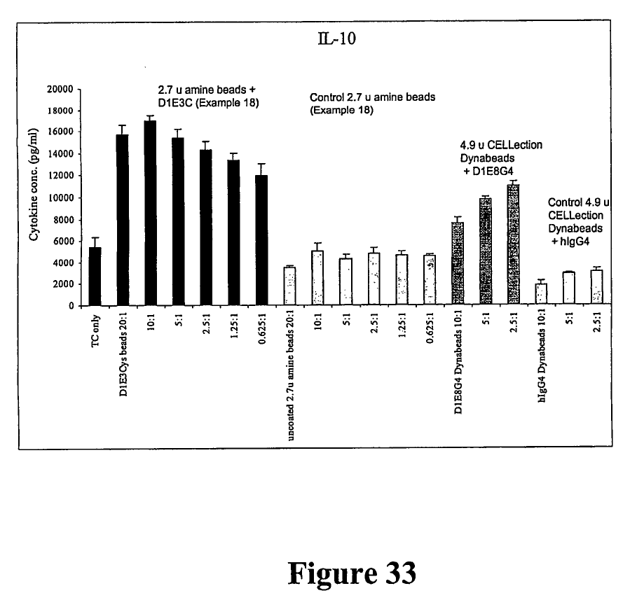

Results for various beads prepared as described above (alongside corresponding uncoated beads as controls) are shown in FIGS. 26 to 33.

### Example 23

### 1 μm, Dynal Beads

MyOne Streptavidin beads (1 μm, Dynal 650.01) and CELLection Biotin Binder (4.5 μm, Dynal 115.21) were coated with anti-hIgG4-Biotin antibodies based on the binding capacity recommended by the supplier.

Briefly, 20 μg of anti-hIgG4-biotin (BD Biosciences) were incubated 30 minutes at room temperature with either 1 mg of MyOne beads (equivalent to 7-12×10⁸ beads) or 10⁸ CELLection beads. The beads were then washed and further incubated with either 100 μg of human Delta1EC domain-hIgG4 fusion protein or 100 μg of hIgG4 (Sigma) for 2 hours at room temperature. After washing, the MyOne beads and the CELLection beads were resuspended in 500 μl of RPMI/BSA 0.1% and stored at 4° C.

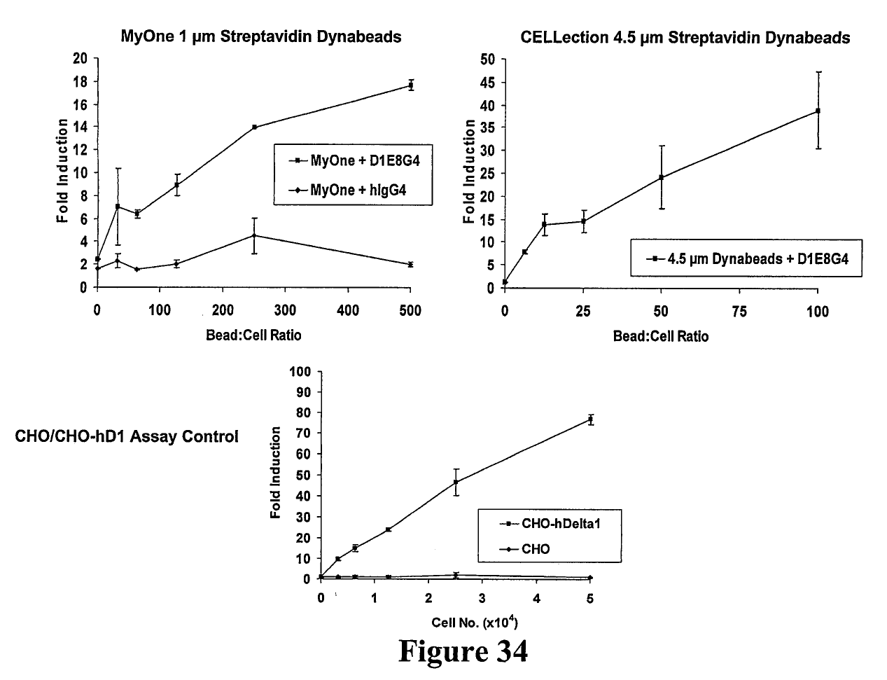

Beads were tested (alongside uncoated beads as controls) in a CHO-N2 reporter assay as described above. Results are shown in FIG. 34.

### Example 24

### Modulation of Cytokine Production by Human CD4+ T Cells in the Presence of Delta1-hIgG4 Immobilised on MyOne or CELLection Dynal Microbeads

Human peripheral blood mononuclear cells (PBMC) were purified from blood using Ficoll-Paque separation medium (Pharmacia). Briefly, 28 ml of blood were overlaid on 21 ml of Ficoll-Paque separation medium and centrifuged at 18-20° C. for 40 minutes at 400 g. PBMC were recovered from the interface and washed 3 times before use for CD4+ T cell purification.

Human CD4+ T cells were isolated by positive selection using anti-CD4 microbeads from Miltenyi Biotech according to the manufacturer's instructions.

The CD4+ T cells were incubated in triplicates in a 96-well-plate (flat bottom) at 10⁵ CD4/well/200 μl in RPMI medium containing 10% FCS, glutamine, penicillin, streptomycin and β₂-mercaptoethanol.

Cytokine production was induced by stimulating the cells with anti-CD3/CD28 T cell expander beads from Dynal at a 1:1 ratio (bead/cell). 10, 5, 2.5, 1.25, 0.62 μl of beads coated with human Delta1EC domain-hIgG4 fusion protein (prepared as described above) or control beads were added in some of the wells. The supernatants were removed after 3 days of incubation at 37° C./5% CO₂/humidified atmosphere and cytokine production was evaluated by ELISA using Pharmingen kits OptEIA Set human IL10 (catalog No. 555157) and OptEIA Set human IL-5 (catalog No. 555202) for IL-10, IL-5 respectively and a human IL-2 DuoSet from R&D Systems (catalog. No DY202) for IL-2 according to the manufacturer's instructions.

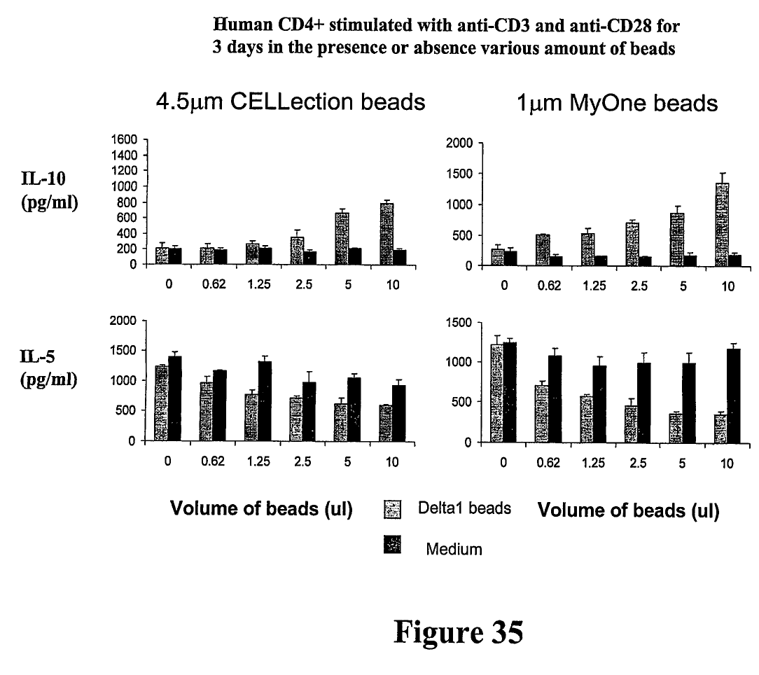

Results are shown in FIG. 35.

### Example 25

### Modulation of Cytokine Production by Delta1-hIgG4 Immobilised on MyOne or CELLection Dynal Microbeads During a Mixed Lymphocyte Reaction

Human peripheral blood mononuclear cells (PBMC) were purified from blood of 2 donors (donor A and donor B) as indicated above.

Human CD14+ monocytes and CD4+ T cells were isolated from PBMC from donor A and B respectively by positive selection using anti-CD14 and anti-CD4 microbeads from Miltenyi Biotech according to the manufacturer's instructions.

The CD14+ cells (donor A) were differentiated into dendritic cells (DC) by incubation for 6 days in medium [RPMI/10% FCS/glutamine/B2-mercaptoethanol/antibiotics] in the presence of hGM-CSF 50 ng/ml and hIL-4 50 ng/ml (both from Peprotech). Dendritic cell maturation was induced by addition into the culture of LPS 1 μg/ml (Sigma L-2654) for the last 24 hours.

Matured-DC were treated for 1 hour with 50 μg/ml Mitomycin C (Sigma) in RPMI and washed 4 times. These cells were then plated at 4×10⁴, 1×10⁴, 2.5×10³, 6.25×10² cells/well in triplicates in a 96-well-plate in RPMI medium containing 10% FCS, glutamine, penicillin, streptomycin and β₂-mercaptoethanol. 2×10⁵ Allogenic CD4+ T cells (donor B) were added into each well given a final volume of 200 μl/well.

10 μl of beads coated with human Delta1EC domain-hIgG4 fusion protein (prepared as described above) or control beads were added in some of the wells.

The supernatants were removed after 5 days of incubation at 37° C./5% CO₂/humidified atmosphere and cytokine production was evaluated by ELISA using Pharmingen kits OptEIA Set human IL10 (catalog No. 555157) and OptEIA Set human IFNg (catalog No 555142) for IL-10 and IFNg respectively and a human TNFa DuoSet from R&D Systems (catalog. No. DY210) for TNFa according to the manufacturer's instructions.

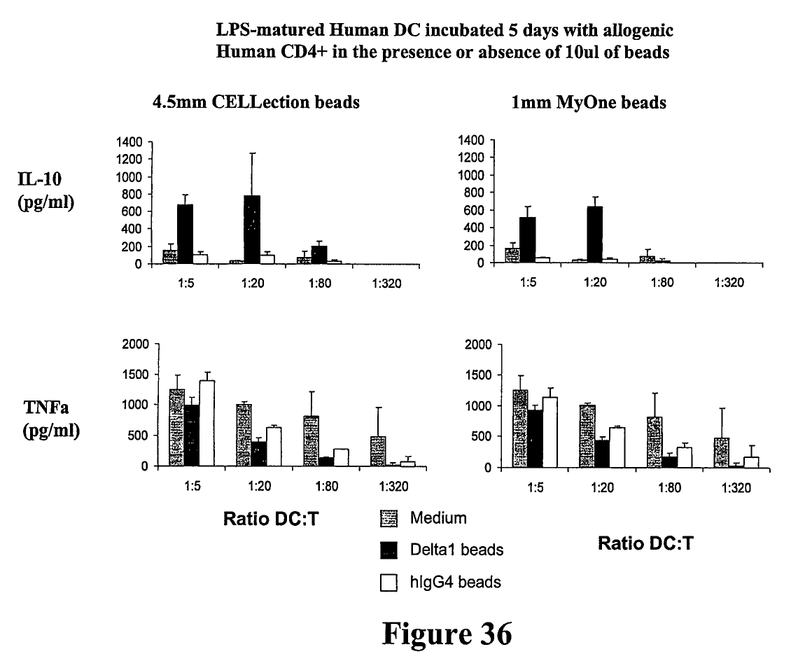

Results are shown in FIG. 36.

The invention is further described by the following numbered paragraphs:

1. A pharmaceutical composition comprising a construct which comprises a multiplicity of bound, linked or immobilised modulators of Notch signalling.

2. A pharmaceutical composition as described in paragraph 1 further comprising a pharmaceutically acceptable diluent or carrier.

3. A pharmaceutical composition as described in paragraph 1 or paragraph 2 wherein the construct comprises at least 3 modulators of Notch signalling which may be the same or different.

4. A pharmaceutical composition as described in paragraph 3 wherein the construct comprises at least about 5 modulators of Notch signalling which may be the same or different.

5. A pharmaceutical composition as described in paragraph 4 wherein the construct comprises at least about 10 modulators of Notch signalling which may be the same or different.

6. A pharmaceutical composition as described in paragraph 5 wherein the construct comprises at least about 100 modulators of Notch signalling which may be the same or different.

7. A pharmaceutical composition as described in any one of the preceding paragraphs wherein the construct comprises a multiplicity of modulators of Notch signalling which may be the same or different bound to a substrate.

8. A pharmaceutical composition as described in paragraph 7 wherein the substrate is a particulate substrate.

9. A pharmaceutical composition as described in paragraph 8 wherein the particulate substrate is a bead.

10. A pharmaceutical composition as described in paragraph 9 wherein the bead is a microbead or microsphere.

11. A pharmaceutical composition as described in paragraph 9 or paragraph 10 wherein the bead has a diameter of from about 0.001 to about 1000 micrometres.

12. A pharmaceutical composition as described in paragraph 11 wherein the bead is a polymeric bead.

13. A pharmaceutical composition as described in paragraph 12 wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

14. A pharmaceutical composition as described in any one of paragraphs 9 to 13 wherein the bead comprises a biodegradable material.

15. A pharmaceutical composition as described in any one of the preceding paragraphs wherein at least one of the modulators of Notch signalling is an activator of a Notch receptor.

16. A pharmaceutical composition as described in paragraph 15 wherein at least one of the modulators of Notch signalling comprises a Notch ligand or a fragment, derivative, homologue, analogue or allelic variant thereof.

17. A pharmaceutical composition as described in paragraph 16 wherein the modulator of Notch signalling comprises Delta or Jagged or a fragment, derivative, homologue, analogue or allelic variant thereof.

18. A pharmaceutical composition as described in any one of the preceding paragraphs wherein at least one of the modulators of the Notch signalling pathway comprises a fusion protein or polypeptide comprising a segment of a Notch ligand extracellular domain and an immunoglobulin Fc segment.

19. A pharmaceutical composition as described in any one of the preceding paragraphs wherein at least one of the modulators of the Notch signalling pathway comprises a protein or polypeptide comprising a Notch ligand DSL domain.

20. A pharmaceutical composition as described in any one of the preceding paragraphs wherein at least one of the modulators of the Notch signalling pathway comprises a protein or polypeptide comprising a Notch ligand DSL domain and from 2 to 20 EGF-like domains.

21. A pharmaceutical composition as described in any one of the preceding paragraphs for therapeutic modulation of Notch signalling.

22. A pharmaceutical composition as described in any one of the preceding paragraphs for modulation of immune cell activity.

23. A pharmaceutical composition as described in any one of the preceding paragraphs for modulation of T-cell activity.

24. A pharmaceutical composition as described in any one of the preceding paragraphs for use in the treatment of inflammation, asthma, allergy, graft rejection, graft-versus-host disease or autoimmune disease.

25. A pharmaceutical composition as described in any one of the preceding paragraphs in sterile form.

26. A method for therapeutic modulation of Notch signalling comprising administering a construct comprising a multiplicity of bound, linked or immobilised modulators of Notch signalling.

27. A method for therapeutic modulation of Notch signalling in immune cells by administering a construct comprising a multiplicity of bound, linked or immobilised modulators of Notch signalling.

28. A method for therapeutic modulation of immune cell activity by administering a construct comprising a multiplicity of bound, linked or immobilised modulators of Notch signalling.

29. A method for therapeutic modulation of T-cell activity by administering a construct comprising a multiplicity of bound, linked or immobilised modulators of Notch signalling.

30. A method for treating inflammation, asthma, allergy, graft rejection, graft-versus-host disease or autoimmune disease by administering a construct comprising a multiplicity of bound, linked or immobilised modulators of Notch signalling.

31. A method as described in any one of paragraphs 26 to 30 wherein the construct comprises at least 3 modulators of Notch signalling which may be the same or different.

32. A method as described in paragraph 31 wherein the construct comprises at least about 5 modulators of Notch signalling which may be the same or different.

33. A method as described in paragraph 32 wherein the construct comprises at least about 10 modulators of Notch signalling which may be the same or different.

34. A method as described in paragraph 32 wherein the construct comprises at least about 100 modulators of Notch signalling which may be the same or different.

35. A method as described in any one of paragraphs 26 to 34 wherein the construct comprises a multiplicity of same or different modulators of Notch signalling bound to a substrate.

36. A method as described in paragraph 35 wherein the substrate is a particulate substrate.

37. A method as described in paragraph 36 wherein the particulate substrate is a bead.

38. A method as described in paragraph 37 wherein the bead is a microbead, nanobead or microsphere or nanosphere.

39. A method as described in paragraph 38 wherein the bead has a diameter of from about 0.001 to about 1000 micrometres.

40. A method as described in paragraph 39 wherein the bead is a polymeric bead.

41. A method as described in paragraph 40 wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

42. A method as described in any one of paragraphs 26 to 41 wherein the modulator of Notch signalling is an activator of a Notch receptor.

43. A method as described in paragraph 42 wherein at least one of the modulators of the Notch signalling pathway comprises a Notch ligand or a fragment, derivative, homologue, analogue or allelic variant thereof.

44. A method as described in paragraph 43 wherein at least one of the modulators of the Notch signalling pathway comprises Delta or Jagged or a fragment, derivative, homologue, analogue or allelic variant thereof.

45. A method as described in paragraph 43 or paragraph 44 wherein at least one of the modulators of the Notch signalling pathway comprises a fusion protein or polypeptide comprising a segment of a Notch ligand extracellular domain and an immunoglobulin Fc segment.

46. A method as described in any one of paragraphs 26 to 45 wherein at least one of the modulators of the Notch signalling pathway comprises a protein or polypeptide comprising a DSL or EGF-like domain.

47. A method as described in paragraph 46 wherein at least one of the modulators of the Notch signalling pathway comprises a protein or polypeptide comprising a Notch ligand DSL domain and from 2 to 20 Notch ligand EGF-like domains or a polynucleotide sequence coding for such a protein or polypeptide.

48. A construct comprising a multiplicity of bound, linked or immobilised modulators of Notch signalling for use in the treatment of disease.

49. A particle bearing a multiplicity of bound, linked or immobilised modulators of Notch signalling for use in the treatment of immune disease.

50. The use of a construct comprising a multiplicity of bound, linked or immobilised modulators of Notch signalling in the manufacture of a medicament for modulation of immune cell activity.

51. A method for treating an immune disorder by administering a construct comprising a multiplicity of bound, linked or immobilised modulators of Notch signalling.

52. A pharmaceutical composition comprising a construct comprising a multiplicity of bound, linked or immobilised modulators of Notch signalling.

53. A pharmaceutical composition comprising a construct comprising a multiplicity of bound, linked or immobilised modulators of Notch signalling and a pharmaceutically acceptable carrier.

54. A substrate bearing a multiplicity of bound modulators of Notch signalling for use in the treatment of disease.

55. A particle bearing a multiplicity of bound modulators of Notch signalling for use in the treatment of disease.

56. A particle comprising a plurality of proteins or polypeptides comprising Delta DSL domains bound to a particulate support matrix.

57. A pharmaceutically acceptable support matrix suitable for in vivo administration which bears a modulator of Notch signalling.

58. A pharmaceutically acceptable support matrix suitable for in vivo administration which bears a plurality of modulators of Notch signalling.

59. A support matrix as described in paragraph 58 in the form of an implantable support matrix.

60. A support matrix as described in paragraph 58 or paragraph 59 in the form of a particle.

61. A support matrix as described in any one of paragraphs 58 to 60 which bears a Notch ligand.

62. A support matrix as described in paragraph 61 which bears a multiplicity of Notch ligand proteins or polypeptides.

63. A method for modulating immune cell activity to treat an immune or inflammatory disorder by contacting an immune cell from a subject with a substrate bearing a multiplicity of bound modulators of Notch signalling.

64. A method for modulating immune cell activity to downregulate an immune response by contacting an immune cell from a subject with a substrate bearing a multiplicity of bound activators of Notch signalling.

65. A method for modulating immune cell activity to upregulate an immune response by contacting an immune cell from a subject with a substrate bearing a multiplicity of bound inhibitors of Notch signalling.

66. A method as described in any one of paragraphs 63 to 65 comprising removing an immune cell from a subject and contacting the immune cell with the substrate ex-vivo.

67. A method as described in any one of paragraphs 63 to 66 comprising the further step of returning the cell to the same or a different subject after contacting the immune cell with the substrate.

68. A method as described in any one of paragraphs 63 to 67 comprising the further step of contacting the immune cell with an antigen or antigenic determinant.

69. A method as described in paragraph 68 comprising contacting the immune cell with an antigen or antigenic determinant presented on a cell surface.

70. A method as described in any one of paragraphs 63 to 69 wherein the immune cell is peripheral immune cell.

71. A method as described in any one of paragraphs 63 to 70 wherein the immune cell is a T-cell, APC, or B-cell.

72. A method as described in any one of paragraphs 63 to 71 wherein the substrate is a particulate substrate.

73. A method as described in paragraph 72 wherein the particulate substrate is a bead.

74. A method as described in paragraph 73 wherein the bead has a diameter of from about 0.001 to about 1000 micrometres.

75. A method as described in paragraph 73 or paragraph 74 wherein the bead is a polymeric bead.

76. A method as described in paragraph 75 wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

77. A method as described in any one of paragraphs 63 to 76 wherein the modulator of Notch signalling is an activator of a Notch receptor.

78. A method as described in paragraph 77 wherein the modulator of Notch signalling comprises a Notch ligand or a fragment, derivative, homologue, analogue or allelic variant thereof.

79. A method as described in paragraph 78 wherein the modulator of Notch signalling comprises Delta or Serrate/Jagged or a fragment, derivative, homologue, analogue or allelic variant thereof.

80. A method as described in paragraph 79 wherein the modulator of the Notch signalling pathway comprises a fusion protein or polypeptide comprising all or part of a Notch ligand extracellular domain and an immunoglobulin F_{c} segment.

81. A method as described in any one of paragraphs 63 to 80 wherein the modulator of the Notch signalling pathway comprises a protein or polypeptide comprising a Notch ligand DSL domain and a Notch ligand EGF-like domain.

82. A method as described in paragraph 81 wherein the modulator of the Notch signalling pathway comprises a protein or polypeptide comprising a Notch ligand DSL domain and from 2 to 20 Notch ligand EGF-like domains.

83. A method as described in any one of paragraphs 63 to 82 wherein the substrate comprises a plate or well.

84. A method as described in any one of paragraphs 63 to 83 wherein one or more of the modulators of Notch signalling comprises a heterologous amino acid sequence.

85. A method as described in paragraph 84 wherein the heterologous amino acid sequence comprises an IgFc domain.

86. A method as described in any one of paragraphs 63 to 85 wherein the immune or inflammatory disorder is allergy, autoimmune disease, cancer, graft rejection, GvHD or an infectious disease.

87. A particle comprising a modulator of Notch signalling bound to a particulate support matrix.

88. A particle as described in paragraph 87 wherein the particulate support matrix is a bead.

89. A particle as described in paragraph 87 or paragraph 88 wherein the modulator of Notch signalling is a Notch ligand.

90. A particle as described in paragraph 89 wherein a plurality of Notch ligands are bound to the particulate support matrix.

91. A particle comprising a modulator of Notch signalling bound to a particulate support matrix.

92. A particle as described in paragraph 87 wherein the particulate support matrix is a bead.

93. A particle as described in paragraph 87 or paragraph 88 wherein the modulator of Notch signalling is a Notch ligand.

94. A particle as described in paragraph 93 wherein a plurality of Notch ligands are bound to the particulate support matrix.

95. A pharmaceutically acceptable support matrix suitable for in vivo administration which bears a modulator of Notch signalling.

96. A support matrix as described in paragraph 95 in the form of an implantable support matrix.

97. A support matrix as described in paragraph 96 in the form of a particle.

98. A support matrix as described in any one of paragraphs 95 to 97 which bears a Notch ligand.

99. A support matrix as described in paragraph 98 which bears a multiplicity of Notch ligands.

100. A protein or polypeptide consisting essentially of the following components:
i) a Notch ligand DSL domain;
ii) 1-5 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

101. A protein or polypeptide consisting essentially of the following components:
i) a Notch ligand DSL domain;
ii) 2-4 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

102. A protein or polypeptide consisting essentially of the following components:
i) a Notch ligand DSL domain;
ii) 2-3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

103. A protein or polypeptide consisting essentially of the following components:
i) a Notch ligand DSL domain;
ii) 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

104. A protein or polypeptide comprising:
i) a Notch ligand DSL domain;
ii) 1-5 and no more than 5 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

105. A protein or polypeptide comprising:
i) a Notch ligand DSL domain;
ii) 2-4 and no more than 4 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

106. A protein or polypeptide comprising:
i) a Notch ligand DSL domain;
ii) 2-3 and no more than 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

107. A protein or polypeptide comprising:
i) a Notch ligand DSL domain;
ii) 3 and no more than 3 Notch ligand EGF domains;
iii) optionally all or part of a Notch ligand N-terminal domain; and
iv) optionally one or more heterologous amino acid sequences;
   and comprising a coupling element suitable for coupling to a support or carrier agent.

108. A protein or polypeptide as described in any one of paragraphs 100 to 107 wherein the coupling agent is suitable for chemical coupling.

109. A protein or polypeptide as described in any one of paragraphs 100 to 107 wherein the coupling agent is suitable for adsorption coupling.

110. A protein or polypeptide as described in any one of paragraphs 100 to 109 wherein the coupling agent is at the C-terminus of the protein or polypeptide.

111. A protein or polypeptide as described any one of paragraphs 100 to 107 wherein the coupling agent is a C-terminal cysteine, aspartate or glutamate residue.

112. A protein or polypeptide as described in any one of paragraphs 100 to 111 wherein DSL and EGF domains are Delta domains.

113. A protein or polypeptide as described in paragraph 112 wherein DSL and EGF domains are human Delta domains.

114. A protein or polypeptide as described in any one of paragraphs 100 to 113 which has at least 50% amino acid sequence similarity to the following sequence along the entire length of the latter:

MGSRCALALAVLSALLCQVWSSGVFELKLQEFVNKKGLLGNRNCCRGGAGPPPCACRTF
FRVCLKHYQASVSPEPPCTYGSAVTPVLGVDSFSLPDGGGADSAFSNPIRFPFGFTWPG
TFSLIIEALHTDSPDDLATENPERLISRLATQRHLTVGEEWSQDLHSSGRTDLKYSYRF
VCDEHYYGEGCSVFCRPRDDAFGHFTCGERGEKVCNPGWKGPYCTEPICLPGCDEQHGF
CDKPGECKCRVGWQGRYCDECIRYPGCLHGTCQQPWQCNCQEGWGGLFCNQDLNYCTHH
KPCKNGATCTNTGQGSYTCSCRPGYTGATCELGIDEC

115. A protein or polypeptide as described in paragraph 114 which has at least 70% amino acid sequence similarity to the sequence of paragraph 114 along the entire length of the latter:

116. A protein or polypeptide as described in paragraph 114 which has at least 90% amino acid sequence similarity to the sequence of paragraph 114 along the entire length of the latter:

117. A polynucleotide coding for a protein or polypeptide as described in any of paragraphs 100 to 116.

118. A pharmaceutically acceptable support matrix bearing a multiplicity of proteins or polypeptides as described in any of paragraphs 100 to 116, said proteins being chemically coupled, affinity coupled or adsorbed onto the matrix.

119. A pharmaceutically acceptable support matrix as described in paragraph 118 which is a particulate support matrix.

120. A pharmaceutically acceptable support matrix as described in paragraph 119 which is a microbead or nanobead.

121. A pharmaceutically acceptable support matrix coupled to a protein or polypeptide as described in any of paragraphs 100 to 116.

122. A support matrix as described in paragraph 121 in particulate form.

123. A bead coupled to a protein or polypeptide as described in any of paragraphs 100 to 116.

124. A bead as described in paragraph 123 which has a diameter of from about 0.001 to about 1000 micrometres.

125. A pharmaceutical composition as described in paragraph 11 wherein the bead is a polymeric bead.

126. A bead as described in any of paragraphs 123 to 125 wherein the bead comprises a biodegradable material.

127. A bead as described in paragraph 125 wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

128. A pharmaceutical composition comprising a bead as described in any one of paragraphs 123 to 127.

### REFERENCES

Tamura et al. (1995) Curr. Biol. 5:1416-1423.
Artavanis-Tsakomas et al. (1995) Science 268:225-232.
Artavanis-Tsakomas et al. (1999) Science 284:770-776.
Lieber et al. (1993) Genes Dev 7(10):1949-65.
Schroeter et al. (1998) Nature 393(6683):382-6.
Struhl et al. (1998) Cell 93(4):649-60.
Weinmaster (2000) Curr. Opin. Genet. Dev. 10:363-369.
Lu et al. (1996) Proc Natl Acad Sci 93(11):5663-7.
Munro and Freeman (2000) Curr. Biol. 10:813-820.
Ju et al. (2000) Nature 405:191-195.
Moloney et al. (2000) Nature 406:369-375.
Brucker et al. (2000) Nature 406:411-415.
Panin et al. (1997) Nature 387:908-912.
Hicks et al. (2000) Nat. Cell. Biol. 2:515-520.
Irvine (1999) Curr. Opin. Genet. Devel. 9:434-441.
Devereux et al. (1984) Nucleic Acid Research 12:87.
Atschul et al. (1990) J. Mol. Biol. 403-410.
Inaba et al. (1992) J. Exp. Med. 175:1157-1167.
Caux et al. (1992) Nature 360:258-261.
Coffin et al. (1998) Gene Therapy 5:718-722.
Kroll et al. (1993) DNA Cell Biol. 12:441-453.
Osborne and Miele (1999) Immunity 11:653-663.
Matsuno et al. (1995) Development 121(8):2633-44.
Matsuno et al. (1998) Nat. Genet. 19:74-78.
Ordentlich et al. (1998) Mol. Cell. Biol. 18:2230-2239.
Takebayashi et al. (1994) J Biol Chem 269(7):150-6.
Leimeister et al. (1999) Mech Dev 85(1-2):173-7.
Maddox (1983) J. Exp. Med. 15(8):121 1
Sauber et al (1995) Virol. 213:439-449
Chee et al. (1996) Science 274:601-614.
Camilli et al. (1994) Proc Natl Acad Sci USA 91:2634-2638.
Hoyne et al. (2000) Immunology 100:281-288.
Hoyne et al. (2001)—reference in press.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

1. A composition comprising (i) a construct which comprises a multiplicity of modulators of Notch signaling that are bound, linked or immobilized to a substrate, and (ii) optionally, a pharmaceutically acceptable diluent or carrier.

2. The composition as claimed in claim 1, wherein the construct comprises at least 3 modulators of Notch signalling which are the same as or different from one another.

3. The composition as claimed in claim 1, wherein the construct comprises at least about 5 modulators of Notch signalling which are the same as or different from one another.

4. The composition as claimed in claim 1, wherein the construct comprises at least about 10 modulators of Notch signalling which are the same as or different from one another.

5. The composition as claimed in claim 1, wherein the construct comprises at least about 100 modulators of Notch signalling which are the same as or different from one another.

6. The composition as claimed in claim 1, wherein the substrate is suitable for in vivo administration.

7. The composition as claimed in claim 1, wherein the substrate is an implantable support matrix.

8. The composition as claimed in claim 1, wherein the substrate is a plate or well.

9. The composition as claimed in claim 1, wherein the substrate is a particle.

10. The composition as claimed in claim 9, wherein the particle is a bead.

11. The composition as claimed in claim 10, wherein the bead is a microbead or microsphere.

12. The composition as claimed in claim 10, wherein the bead has a diameter of from about 0.001 to about 1000 micrometres.

13. The composition as claimed in claim 10, wherein the bead is a polymeric bead.

14. The composition as claimed in claim 10, wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

15. The composition as claimed in claim 10, wherein the bead comprises a biodegradable material.

16. The composition as claimed in claim 1, wherein at least one of the modulators of Notch signalling is an activator of a Notch receptor.

17. The composition as claimed in claim 1, wherein at least one of the modulators of Notch signalling comprises a Notch ligand or a fragment, derivative, homologue, analogue or allelic variant thereof.

18. The composition as claimed in claim 17, wherein the Notch ligand is Delta or Jagged.

19. The composition as claimed in claim 1, wherein at least one of the modulators of Notch signalling comprises a heterologous amino acid sequence.

20. The composition as claimed in claim 1, wherein the heterologous amino acid sequence comprises an immunoglobulin Fc domain.

21. The composition as claimed in claim 1, wherein at least one of the modulators of Notch signalling comprises a fusion protein or polypeptide comprising a segment of a Notch ligand extracellular domain and an immunoglobulin Fc segment.

22. The composition as claimed in claim 1, wherein at least one of the modulators of the Notch signalling pathway comprises a protein or polypeptide comprising a Notch ligand DSL domain.

23. The composition as claimed in claim 1, wherein at least one of the modulators of the Notch signalling pathway comprises a protein or polypeptide comprising a Notch ligand DSL domain and from 2 to 20 EGF-like domains.

24. A method for modulating Notch signalling in immune cells comprising contacting the immune cells with the composition as claimed in claim 1.

25. A method for treating an immune or inflammatory disorder by administering the composition as claimed in claim 1 to a subject in need thereof.

26. The method as claimed in claim 25, wherein the immune or inflammatory disorder is selected from the group consisting of asthma, allergy, autoimmune disease, cancer, graft rejection, graft-versus-host disease, infectious disease and inflammation.

27. A method for modulating immune cell activity comprising contacting the immune cell with the composition as claimed in claim 1.

28. The method of claim 27, comprising removing the immune cell from a subject and contacting the immune cell with the composition ex-vivo.

29. The method as claimed in claim 28, further comprising returning the immune cell to the same or a different subject after contacting the immune cell with the composition.

30. The method as claimed in claim 27, further comprising contacting the immune cell with an antigen or antigenic determinant

31. The method as claimed in claim 30, wherein the antigen or antigenic determinant is presented on a cell surface.

32. The method as claimed in claim 27, wherein the immune cell is peripheral immune cell.

33. The method as claimed in claim 27, wherein the immune cell is a T-cell, an antigen presenting cell (APC), or a B-cell.

34. A protein or polypeptide comprising:
i) a Notch ligand DSL domain;
ii) at least one and no more than five Notch ligand EGF domains;
iiii) optionally, all or part of a Notch ligand N-terminal domain; and
iv) optionally, one or more heterologous amino acid sequences; and
v) a coupling element suitable for coupling to a support or carrier agent.

35. The protein or polypeptide as claimed in claim 34, comprising at least two and no more than four Notch ligand EGF.

36. The protein or polypeptide as claimed in claim 34, comprising at least two and no more than three Notch ligand EGF.

37. The protein or polypeptide as claimed in claim 34, comprising at least three and no more than three Notch ligand EGF domains:

38. The protein or polypeptide as claimed in claim 34, wherein the coupling agent is suitable for adsorption coupling.

39. The protein or polypeptide as claimed in claim 34, wherein the coupling agent is at the C-terminus of the protein or polypeptide.

40. The protein or polypeptide as claimed in claim 34, wherein the coupling agent is a C-terminal cysteine, aspartate or glutamate residue.

41. The protein or polypeptide as claimed in claim 34, wherein the DSL and EGF domains are Delta domains.

42. The protein or polypeptide as claimed in claim 41, wherein DSL and EGF domains are human Delta domains.

43. The protein or polypeptide as claimed in claim 34, which has at least 50% amino acid sequence similarity to SEQ ID NO:1 along the entire length of SEQ ID NO:1.

44. The protein or polypeptide as claimed in claim 34, which has at least 70% amino acid sequence similarity to SEQ ID NO:1 along the entire length of SEQ ID NO:1.

45. The protein or polypeptide as claimed in claim 34, which has at least 90% amino acid sequence similarity to SEQ ID NO:1 along the entire length of SEQ ID NO:1.

46. A polynucleotide encoding the protein or polypeptide as claimed in claim 34.

47. A pharmaceutically acceptable support matrix coupled to the protein or polypeptide as claimed in claim 34, wherein the protein or polypeptide is chemically coupled, affinity coupled or adsorbed onto the matrix.

48. The support matrix as claimed in claim 47, which is a particle.

49. The support matrix as claimed in claim 47, which is a bead.

50. The support matrix as claimed in claim 49, which is a microbead or nanobead.

51. The support matrix as claimed in claim 49, which has a diameter of from about 0.001 to about 1000 micrometres.

52. The support matrix as claimed in claim 49, wherein the bead is a polymeric bead.

53. The support matrix as claimed in claim 49, wherein the bead comprises polystyrene, polyacrylamide, latex, cellulose, silica, dextran, agarose, cellulose, polylactide, or poly(methylmethacrylate) (PMMA) optionally in modified, crosslinked or derivatized form.

54. The support matrix as claimed in claim 49, wherein the bead comprises a biodegradable material.

55. A pharmaceutical composition comprising the support matrix as claimed in claim 47.

56. A protein or polypeptide consisting essentially of:
i) a Notch ligand DSL domain;
ii) 1-5 Notch ligand EGF domains;
iii) optionally, all or part of a Notch ligand N-terminal domain; and
iv) optionally, one or more heterologous amino acid sequences; and
v) a coupling element suitable for coupling to a support or carrier agent.
